SEQUENCE LISTING (135 sequences)

SEQ ID NO: 1 - protein, 127 aa, Artificial Sequence
  AB12 VH
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Artificial Sequence
  AB12 VL
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Artificial Sequence
  AB12 VH CDR1
RYIIN

SEQ ID NO: 4 - protein, 17 aa, Artificial Sequence
  AB12 VH CDR2
RIIPILGVENYAQKFQG

SEQ ID NO: 5 - protein, 6 aa, Artificial Sequence
  AB12 VH CDR3
KDWFDY

SEQ ID NO: 6 - protein, 11 aa, Artificial Sequence
  AB12 VL CDR1
RASQSVSSYLA

SEQ ID NO: 7 - protein, 7 aa, Artificial Sequence
  AB12 VL CDR2
GASSRAT

SEQ ID NO: 8 - protein, 9 aa, Artificial Sequence
  AB12 VL CDR3
QQYGSSPLT

SEQ ID NO: 9-10 - skipped (2 entries)

SEQ ID NO: 11 - protein, 119 aa, Artificial Sequence
  RB4v1.0 VH
QVQLVQPGAEVVKPGASVKLSCKTSGYTFTSNWMHWVKQRPGQGLEWIGEIDPSDSYTNYNQNFKGKAKLTVDKSTSTAYMEVSSLRSDDTAVYYCARGSNPYYYAMDYWGQGTSVTVS

SEQ ID NO: 12 - protein, 119 aa, Artificial Sequence
  RB4v1.2 VH
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVS

SEQ ID NO: 13 - protein, 104 aa, Artificial Sequence
  RB4v1.0 VK
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 14 - protein, 104 aa, Artificial Sequence; identical to SEQ ID NO: 13
  RB4v1.2 VK

SEQ ID NO: 15 - protein, 109 aa, Artificial Sequence
  Epratuzumab VH
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQG

SEQ ID NO: 16 - protein, 106 aa, Artificial Sequence
  Epratuzumab VL
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQG

SEQ ID NO: 17 - protein, 445 aa, Artificial Sequence
  EMabC220-HC
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 219 aa, Artificial Sequence
  EMabC220-LC
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 19-20 - skipped (2 entries)

SEQ ID NO: 21 - protein, 115 aa, Artificial Sequence
  J591 VH
EVQLQQSGPELKKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 22 - protein, 107 aa, Artificial Sequence
  J591 VL
DIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLK

SEQ ID NO: 23 - protein, 115 aa, Artificial Sequence
  J591 VH DeIm
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 24 - protein, 107 aa, Artificial Sequence
  J591 VK DeIm
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 25 - protein, 444 aa, Artificial Sequence
  J591BJ DeIm heavy chain
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 214 aa, Artificial Sequence
  J591BJ DeIm light chain
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 27-30 - skipped (4 entries)

SEQ ID NO: 31 - protein, 122 aa, Artificial Sequence
  1H12 VH
QVQLVESGGGVVQPGRSLRLSCAASGFTFSSYGMSWVRQAPGKGLEWVATISSGGSYTYYPDSVKGRFTISRDNSKNTLYLQMNSLRAEDTAVYYCARHPIYYTYDDTMDYWGQGTTVTVSS

SEQ ID NO: 32 - protein, 108 aa, Artificial Sequence
  1H12 VL
EIVLTQSPGTLSLSPGERATLSCSASSSVSSGNFHWYQQKPGLAPRLLIYRTSNLASGIPARFSGSGSGTDFTLTISSLEPEDFAVYYCQQWSGYPWTFGGGTKLEIK

SEQ ID NO: 33 - protein, 451 aa, Artificial Sequence
  1H12 Heavy Chain
  MISC_FEATURE (302)..(302): Xaa = Asn297 (numbering according to Kabat)
QVQLVESGGGVVQPGRSLRLSCAASGFTFSSYGMSWVRQAPGKGLEWVATISSGGSYTYYPDSVKGRFTISRDNSKNTLYLQMNSLRAEDTAVYYCARHPIYYTYDDTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 215 aa, Artificial Sequence
  1H12 Light Chain
EIVLTQSPGTLSLSPGERATLSCSASSSVSSGNFHWYQQKPGLAPRLLIYRTSNLASGIPARFSGSGSGTDFTLTISSLEPEDFAVYYCQQWSGYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, Artificial Sequence
  1H12 VH CDR1
SYGMS

SEQ ID NO: 36 - protein, 17 aa, Artificial Sequence
  1H12 VH CDR2
TISSGGSYTYYPDSVKG

SEQ ID NO: 37 - protein, 13 aa, Artificial Sequence
  1H12 VH CDR3
HPIYYTYDDTMDY

SEQ ID NO: 38 - protein, 12 aa, Artificial Sequence
  1H12 VL CDR1
SASSSVSSGNFH

SEQ ID NO: 39 - protein, 7 aa, Artificial Sequence
  1H12 VL CDR2
RTSNLAS

SEQ ID NO: 40 - protein, 9 aa, Artificial Sequence
  1H12 VL CDR3
QQWSGYPWT

SEQ ID NO: 41 - protein, 121 aa, Artificial Sequence
  HuBa-1-3d VH
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSS

SEQ ID NO: 42 - protein, 113 aa, Artificial Sequence
  HuBa-1-3d VL
DIVMTQSPDSLAVSLGERATINCKSSQSLLNSSNQKNYLAWYQQKPGQPPKLLVYFASTRESGVPDRFSGSGSGTDFTLTISSLQAEDVAVYYCQQHYSTPPTFGQGTKLEIK

SEQ ID NO: 43 - protein, 450 aa, Artificial Sequence
  HuBa-1-3d Heavy Chain
  MISC_FEATURE (301)..(301): Xaa = Asn297
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 220 aa, Artificial Sequence
  HuBa-1-3d Light Chain
DIVMTQSPDSLAVSLGERATINCKSSQSLLNSSNQKNYLAWYQQKPGQPPKLLVYFASTRESGVPDRFSGSGSGTDFTLTISSLQAEDVAVYYCQQHYSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 5 aa, Artificial Sequence
  HuBa-1-3d VH CDR1
DYAMH

SEQ ID NO: 46 - protein, 17 aa, Artificial Sequence
  HuBa-1-3d VH CDR2
VISTYYGNTNYNQKFKG

SEQ ID NO: 47 - protein, 12 aa, Artificial Sequence
  HuBa-1-3d VH CDR3
GGLREYYYAMDY

SEQ ID NO: 48 - protein, 17 aa, Artificial Sequence
  HuBa-1-3d VL CDR1
KSSQSLLNSSNQKNYLA

SEQ ID NO: 49 - protein, 7 aa, Artificial Sequence
  HuBa-1-3d VL CDR2
FASTRES

SEQ ID NO: 50 - protein, 9 aa, Artificial Sequence
  HuBa-1-3d VL CDR3
QQHYSTPPT

SEQ ID NO: 51 - protein, 451 aa, Artificial Sequence
  HuBa-1-3d Heavy Chain, terminal K
  MISC_FEATURE (301)..(301): Xaa = Asn297
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52-60 - skipped (9 entries)

SEQ ID NO: 61 - protein, 116 aa, Artificial Sequence
  3A4 VH
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSS

SEQ ID NO: 62 - protein, 112 aa, Artificial Sequence
  3A4 VL
DIVMTQTPLSLPVTPGEPASISCRSSQSLLHSNGNTYLEWYLQKPGQSPQLLIYTVSNRFSGVPDRFSGSGSGTDFTLKISRVEAEDVGVYYCFQGSHVPLTFGQGTKLEIK

SEQ ID NO: 63 - protein, 445 aa, Artificial Sequence
  3A4 Heavy Chain
  MISC_FEATURE (296)..(297): Xaa = Asn297
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 219 aa, Artificial Sequence
  3A4 Light Chain
DIVMTQTPLSLPVTPGEPASISCRSSQSLLHSNGNTYLEWYLQKPGQSPQLLIYTVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 10 aa, Artificial Sequence
  3A4 VH CDR1
GYTFTDDYMS

SEQ ID NO: 66 - protein, 10 aa, Artificial Sequence
  3A4 VH CDR2
DINPYNGDTN

SEQ ID NO: 67 - protein, 7 aa, Artificial Sequence
  3A4 VH CDR3
DPGAMDY

SEQ ID NO: 68 - protein, 16 aa, Artificial Sequence
  3A4 VL CDR1
RSSQSLLHSNGNTYLE

SEQ ID NO: 69 - protein, 7 aa, Artificial Sequence
  3A4 VL CDR2
TVSNRFS

SEQ ID NO: 70 - protein, 9 aa, Artificial Sequence
  3A4 VL CDR3
FQGSHVPLT

SEQ ID NO: 71 - protein, 446 aa, Artificial Sequence
  3A4 Heavy Chain, terminal K
  MISC_FEATURE (296)..(296): Xaa = Asn297
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 84 aa, Homo sapiens
MDDDAAPRVEGVPVAVHKHALHDGLRQVAGPGAAAAHLPRWPPPQLAASRREAPPLSQRPHRTQGAGSPPETNEKLTNPQVKEK

SEQ ID NO: 73 - protein, 112 aa, Artificial Sequence
  3A4-L2 VL
DVVMTQTPLSLPVTPGEPASISCRSSQSLLHSNGNTYLEWYLQKPGQSPKLLIYTVSNRFSGVPDRFSGSGSGTDFTLKISRVEAEDVGVYYCFQGSHVPLTFGQGTKLEIK

SEQ ID NO: 74 - protein, 219 aa, Artificial Sequence
  3A4-L2 Light Chain
DVVMTQTPLSLPVTPGEPASISCRSSQSLLHSNGNTYLEWYLQKPGQSPKLLIYTVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 112 aa, Artificial Sequence
  3A4-K4 VL
DIVMTQSPDSLAVSLGERATINCRSSQSLLHSNGNTYLEWYQQKPGQPPKLLIYTVSNRFSGVPDRFSGSGSGTDFTLTISSLQAEDVAVYYCFQGSHVPLTFGQGTKVEIK

SEQ ID NO: 76 - protein, 219 aa, Artificial Sequence
  3A4-K4 Light Chain
DIVMTQSPDSLAVSLGERATINCRSSQSLLHSNGNTYLEWYQQKPGQPPKLLIYTVSNRFSGVPDRFSGSGSGTDFTLTISSLQAEDVAVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77-80 - skipped (4 entries)

SEQ ID NO: 81 - protein, 122 aa, Artificial Sequence
  XA4 VH
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSS

SEQ ID NO: 82 - protein, 107 aa, Artificial Sequence
  XA4 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 83 - protein, 451 aa, Artificial Sequence
  XA4 Heavy Chain
  MISC_FEATURE (302)..(302): Xaa = Asn297
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 214 aa, Artificial Sequence
  XA4 Light Chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85-90 - skipped (6 entries)

SEQ ID NO: 91 - protein, 452 aa, Artificial Sequence
  XA4 Heavy Chain, terminal K
  MISC_FEATURE (302)..(302): Xaa = Asn297
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 119 aa, Artificial Sequence
  XFT VH
QVELVQSGAVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 93 - protein, 111 aa, Artificial Sequence
  XFT VL
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLEIK

SEQ ID NO: 94 - protein, 448 aa, Artificial Sequence
  XFT Heavy Chain
  MISC_FEATURE (299)..(299): Xaa = Asn297
QVELVQSGAVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 218 aa, Artificial Sequence
  XFT Light Chain
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96-101 - skipped (6 entries)

SEQ ID NO: 102 - protein, 449 aa, Artificial Sequence
  XFT Heavy Chain, terminal K
  MISC_FEATURE (299)..(299): Xaa = Asn297
QVELVQSGAVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 119 aa, Artificial Sequence
  X09 VH
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 104 - protein, 106 aa, Artificial Sequence
  X09 VL
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 105 - protein, 448 aa, Artificial Sequence
  X09 Heavy Chain
  MISC_FEATURE (299)..(299): Xaa = Asn297
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 213 aa, Artificial Sequence
  X09 Light Chain
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107-112 - skipped (6 entries)

SEQ ID NO: 113 - protein, 449 aa, Artificial Sequence
  X09 Heavy Chain, terminal K
  MISC_FEATURE (299)..(299): Xaa = Asn297
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 119 aa, Artificial Sequence
  X09.2 VH
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 115 - protein, 106 aa, Artificial Sequence
  X09.2 VL
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 116 - protein, 448 aa, Artificial Sequence; identical to SEQ ID NO: 105
  X09.2 Heavy Chain
  MISC_FEATURE (299)..(299): Xaa = Asn297

SEQ ID NO: 117 - protein, 213 aa, Artificial Sequence; identical to SEQ ID NO: 106
  X09.2 Light Chain

SEQ ID NO: 118 - protein, 449 aa, Artificial Sequence; identical to SEQ ID NO: 113
  X09.2 Heavy Chain, terminal K
  MISC_FEATURE (299)..(299): Xaa = Asn297

SEQ ID NO: 119 - protein, 655 aa, Artificial Sequence
  ATP-binding cassette sub-family G member 2 UniProt record number: Q9UNQ0
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 120 - protein, 1545 aa, Artificial Sequence
  ATP-binding cassette sub-family C (MRP) member 2 or Canalicular multispecific organic anion transporter 1 UniProt record number: Q92887
MLEKFCNSTFWNSSFLDSPEADLPLCFEQTVLVWIPLGYLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 121 - protein, 709 aa, Artificial Sequence
  Solute carrier organic anion transporter fam. mem. 2B1 UniProt record number: O94956
MGPRIGPAGEVPQVPDKETKATMGTENTPGGKASPDPQDVRPSVFHNIKLFVLCHSLLQLAQLMISGYLKSSISTVEKRFGLSSQTSGLLASFNEVGNTALIVFVSYFGSRVHRPRMIGYGAILVALAGLLMTLPHFISEPYRYDNTSPEDMPQDFKASLCLPTTSAPASAPSNGNCSSYTETQHLSVVGIMFVAQTLLGVGGVPIQPFGISYIDDFAHNSNSPLYLGILFAVTMMGPGLAFGLGSLMLRLYVDINQMPEGGISLTIKDPRWVGAWWLGFLIAAGAVALAAIPYFFFPKEMPKEKRELQFRRKVLAVTDSPARKGKDSPSKQSPGESTKKQDGLVQIAPNLTVIQFIKVFPRVLLQTLRHPIFLLVVLSQVCLSSMAAGMAIFLPKFLERQFSITASYANLLIGCLSFPSVIVGIVVGGVLVKRLHLGPVGCGALCLLGMLLCLFFSLPLFFIGCSSHQIAGITHQTSAHPGLELSPSCMEACSCPLDGFNPVCDPSTRVEYITPCHAGCSSWVVQDALDNSQVFYTNCSCVVEGNPVLAGSCDSTCSHLVVPFLLLVSLGSALACLTHTPSFMLILRGVKKEDKTLAVGIQFMFLRILAWMPSPVIHGSAIDTTCVHWALSCGRRAVCRYYNNDLLRNRFIGLQFFFKTGSVICFALVLAVLRQQDKEARTKESRSSPAVEQQLLVSGPGKKPEDSRV

SEQ ID NO: 122 - protein, 511 aa, Artificial Sequence
  Solute carrier family 7, number 7 or Y+L amino acid transporter 1 UniProt record number: Q9UM01
MVDSTEYEVASQPEVETSPLGDGASPGPEQVKLKKEISLLNGVCLIVGNMIGSGIFVSPKGVLIYSASFGLSLVIWAVGGLFSVFGALCYAELGTTIKKSGASYAYILEAFGGFLAFIRLWTSLLIIEPTSQAIIAITFANYMVQPLFPSCFAPYAASRLLAAACICLLTFINCAYVKWGTLVQDIFTYAKVLALIAVIVAGIVRLGQGASTHFENSFEGSSFAVGDIALALYSALFSYSGWDTLNYVTEEIKNPERNLPLSIGISMPIVTIIYILTNVAYYTVLDMRDILASDAVAVTFADQIFGIFNWIIPLSVALSCFGGLNASIVAASRLFFVGSREGHLPDAICMIHVERFTPVPSLLFNGIMALIYLCVEDIFQLINYYSFSYWFFVGLSIVGQLYLRWKEPDRPRPLKLSVFFPIVFCLCTIFLVAVPLYSDTINSLIGIAIALSGLPFYFLIIRVPEHKRPLYLRRIVGSATRYLQVLCMSVAAEMDLEDGGEMPKQRDPKSN

SEQ ID NO: 123 - protein, 556 aa, Artificial Sequence
  Solute carrier family 22 member 3 UniProt record number: O75751
MPSFDEALQRVGEFGRFQRRVFLLLCLTGVTFAFLFVGVVFLGTQPDHYWCRGPSAAALAERCGWSPEEEWNRTAPASRGPEPPERRGRCQRYLLEAANDSASATSALSCADPLAAFPNRSAPLVPCRGGWRYAQAHSTIVSEFDLVCVNAWMLDLTQAILNLGFLTGAFTLGYAADRYGRIVIYLLSCLGVGVTGVVVAFAPNFPVFVIFRFLQGVFGKGTWMTCYVIVTEIVGSKQRRIVGIVIQMFFTLGIIILPGIAYFIPNWQGIQLAITLPSFLFLLYYWVVPESPRWLITRKKGDKALQILRRIAKCNGKYLSSNYSEITVTDEEVSNPSFLDLVRTPQMRKCTLILMFAWFTSAVVYQGLVMRLGIIGGNLYIDFFISGVVELPGALLILLTIERLGRRLPFAASNIVAGVACLVTAFLPEGIAWLRTTVATLGRLGITMAFEIVYLVNSELYPTTLRNFGVSLCSGLCDFGGIIAPFLLFRLAAVWLELPLIIFGILASICGGLVMLLPETKGIALPETVDDVEKLGSPHSCKCGRNKKTPVSRSHL

SEQ ID NO: 124 - protein, 643 aa, Artificial Sequence [sequence omitted]

SEQ ID NO: 125 - protein, 1359 aa, Artificial Sequence
  ATP-binding cassette sub-family C (MRP) member 12 or Multidrug resistance-associated protein 9 UniProt record number: Q96J65
MVGEGPYLISDLDQRGRRRSFAERYDPSLKTMIPVRPCARLAPNPVDDAGLLSFATFSWLTPVMVKGYRQRLTVDTLPPLSTYDSSDTNAKRFRVLWDEEVARVGPEKASLSHVVWKFQRTRVLMDIVANILCIIMAAIGPVILIHQILQQTERTSGKVWVGIGLCIALFATEFTKVFFWALAWAINYRTAIRLKVALSTLVFENLVSFKTLTHISVGEVLNILSSDSYSLFEAALFCPLPATIPILMVFCAAYAFFILGPTALIGISVYVIFIPVQMFMAKLNSAFRRSAILVTDKRVQTMNEFLTCIRLIKMYAWEKSFTNTIQDIRRRERKLLEKAGFVQSGNSALAPIVSTIAIVLTLSCHILLRRKLTAPVAFSVIAMFNVMKFSIAILPFSIKAMAEANVSLRRMKKILIDKSPPSYITQPEDPDTVLLLANATLTWEHEASRKSTPKKLQNQKRHLCKKQRSEAYSERSPPAKGATGPEEQSDSLKSVLHSISFVVRKGKILGICGNVGSGKSSLLAALLGQMQLQKGVVAVNGTLAYVSQQAWIFHGNVRENILFGEKYDHQRYQHTVRVCGLQKDLSNLPYGDLTEIGERGLNLSGGQRQRISLARAVYSDRQLYLLDDPLSAVDAHVGKHVFEECIKKTLRGKTVVLVTHQLQFLESCDEVILLEDGEICEKGTHKELMEERGRYAKLIHNLRGLQFKDPEHLYNAAMVEAFKESPAEREEDAGIIVLAPGNEKDEGKESETGSEFVDTKVPEHQLIQTESPQEGTVTWKTYHTYIKASGGYLLSLFTVFLFLLMIGSAAFSNWWLGLWLDKGSRMTCGPQGNRTMCEVGAVLADIGQHVYQWVYTASMVFMLVFGVTKGFVFTKTTLMASSSLHDTVFDKILKSPMSFFDTTPTGRLMNRFSKDMDELDVRLPFHAENFLQQFFMVVFILVILAAVFPAVLLVVASLAVGFFILLRIFHRGVQELKKVENVSRSPWFTHITSSMQGLGIIHAYGKKESCITYHLLYFNCALRWFALRMDVLMNILTFTVALLVTLSFSSISTSSKGLSLSYIIQLSGLLQVCVRTGTETQAKFTSVELLREYISTCVPECTHPLKVGTCPKDWPSRGEITFRDYQMRYRDNTPLVLDSLNLNIQSGQTVGIVGRTGSGKSSLGMALFRLVEPASGTIFIDEVDICILSLEDLRTKLTVIPQDPVLFVGTVRYNLDPFESHTDEMLWQVLERTFMRDTIMKLPEKLQAEVTENGENFSVGERQLLCVARALLRNSKIILLDEATASMDSKTDTLVQNTIKDAFKGCTVLTIAHRLNTVLNCDHVLVMENGKVIEFDKPEVLAEKPDSAFAMLLAAEVRL

SEQ ID NO: 126 - protein, 1500 aa, Artificial Sequence
  ATPase, Cu++ transporting alpha polypeptide or Copper-transporting ATPase 1 UniProt record number: Q04656
MDPSMGVNSVTISVEGMTCNSCVWTIEQQIGKVNGVHHIKVSLEEKNATIIYDPKLQTPKTLQEAIDDMGFDAVIHNPDPLPVLTDTLFLTVTASLTLPWDHIQSTLLKTKGVTDIKIYPQKRTVAVTIIPSIVNANQIKELVPELSLDTGTLEKKSGACEDHSMAQAGEVVLKMKVEGMTCHSCTSTIEGKIGKLQGVQRIKVSLDNQEATIVYQPHLISVEEMKKQIEAMGFPAFVKKQPKYLKLGAIDVERLKNTPVKSSEGSQQRSPSYTNDSTATFIIDGMHCKSCVSNIESTLSALQYVSSIVVSLENRSAIVKYNASSVTPESLRKAIEAVSPGLYRVSITSEVESTSNSPSSSSLQKIPLNVVSQPLTQETVINIDGMTCNSCVQSIEGVISKKPGVKSIRVSLANSNGTVEYDPLLTSPETLRGAIEDMGFDATLSDTNEPLVVIAQPSSEMPLLTSTNEFYTKGMTPVQDKEEGKNSSKCYIQVTGMTCASCVANIERNLRREEGIYSILVALMAGKAEVRYNPAVIQPPMIAEFIRELGFGATVIENADEGDGVLELVVRGMTCASCVHKIESSLTKHRGILYCSVALATNKAHIKYDPEIIGPRDIIHTIESLGFEASLVKKDRSASHLDHKREIRQWRRSFLVSLFFCIPVMGLMIYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLFLKLYRKPTYESYELPARSQIGQKSPSEISVHVGIDDTSRNSPKLGLLDRIVNYSRASINSLLSDKRSLNSVVTSEPDKHSLLVGDFREDDDTAL

SEQ ID NO: 127 - protein, 342 aa, Artificial Sequence
  Aquaporin-7 UniProt record number: O14520
MVQASGHRRSTRGSKMVSWSVIAKIQEILQRKMVREFLAEFMSTYVMMVFGLGSVAHMVLNKKYGSYLGVNLGFGFGVTMGVHVAGRISGAHMNAAVTFANCALGRVPWRKFPVYVLGQFLGSFLAAATIYSLFYTAILHFSGGQLMVTGPVATAGIFATYLPDHMTLWRGFLNEAWLTGMLQLCLFAITDQENNPALPGTEALVIGILVVIIGVSLGMNTGYAINPSRDLPPRIFTFIAGWGKQVFSNGENWWWVPVVAPLLGAYLGGIIYLVFIGSTIPREPLKLEDSVAYEDHGITVLPKMGSHEPTISPLTPVSVSPANRSSVHPAPPLHESMALEHF

SEQ ID NO: 128 - protein, 664 aa, Artificial Sequence
  Solute carrier family 5 member 1 or Sodium/glucose cotransporter 1 UniProt record number: P13866
MDSSTWSPKTTAVTRPVETHELIRNAADISIIVIYFVVVMAVGLWAMFSTNRGTVGGFFLAGRSMVWWPIGASLFASNIGSGHFVGLAGTGAASGIAIGGFEWNALVLVVVLGWLFVPIYIKAGVVTMPEYLRKRFGGQRIQVYLSLLSLLLYIFTKISADIFSGAIFINLALGLNLYLAIFLLLAITALYTITGGLAAVIYTDTLQTVIMLVGSLILTGFAFHEVGGYDAFMEKYMKAIPTIVSDGNTTFQEKCYTPRADSFHIFRDPLTGDLPWPGFIFGMSILTLWYWCTDQVIVQRCLSAKNMSHVKGGCILCGYLKLMPMFIMVMPGMISRILYTEKIACVVPSECEKYCGTKVGCTNIAYPTLVVELMPNGLRGLMLSVMLASLMSSLTSIFNSASTLFTMDIYAKVRKRASEKELMIAGRLFILVLIGISIAWVPIVQSAQSGQLFDYIQSITSYLGPPIAAVFLLAIFWKRVNEPGAFWGLILGLLIGISRMITEFAYGTGSCMEPSNCPTIICGVHYLYFAIILFAISFITIVVISLLTKPIPDVHLYRLCWSLRNSKEERIDLDAEEENIQEGPKETIEIETQVPEKKKGIFRRAYDLFCGLEQHGAPKMTEEEEKAMKMKMTDTSEKPLWRTVLNVNGIILVTVAVFCHAYFA

SEQ ID NO: 129 - protein, 539 aa, Artificial Sequence
  Solute carrier family 16 member 2 or Monocarboxylate transporter 8 UniProt record number: P36021
MALQSQASEEAKGPWQEADQEQQEPVGSPEPESEPEPEPEPEPVPVPPPEPQPEPQPLPDPAPLPELEFESERVHEPEPTPTVETRGTARGFQPPEGGFGWVVVFAATWCNGSIFGIHNSVGILYSMLLEEEKEKNRQVEFQAAWVGALAMGMIFFCSPIVSIFTDRLGCRITATAGAAVAFIGLHTSSFTSSLSLRYFTYGILFGCGCSFAFQPSLVILGHYFQRRLGLANGVVSAGSSIFSMSFPFLIRMLGDKIKLAQTFQVLSTFMFVLMLLSLTYRPLLPSSQDTPSKRGVRTLHQRFLAQLRKYFNMRVFRQRTYRIWAFGIAAAALGYFVPYVHLMKYVEEEFSEIKETWVLLVCIGATSGLGRLVSGHISDSIPGLKKIYLQVLSFLLLGLMSMMIPLCRDFGGLIVVCLFLGLCDGFFITIMAPIAFELVGPMQASQAIGYLLGMMALPMIAGPPIAGLLRNCFGDYHVAFYFAGVPPIIGAVILFFVPLMHQRMFKKEQRDSSKDKMLAPDPDPNGELLPGSPNPEEPI

SEQ ID NO: 130 - protein, 487 aa, Artificial Sequence
  Solute carrier family 7 member 9 or b(0,+)-type amino acid transporter 1 UniProt record number: P82251
MGDTGLRKRREDEKSIQSQEPKTTSLQKELGLISGISIIVGTIIGSGIFVSPKSVLSNTEAVGPCLIIWAACGVLATLGALCFAELGTMITKSGGEYPYLMEAYGPIPAYLFSWASLIVIKPTSFAIICLSFSEYVCAPFYVGCKPPQIVVKCLAAAAILFISTVNSLSVRLGSYVQNIFTAAKLVIVAIIIISGLVLLAQGNTKNFDNSFEGAQLSVGAISLAFYNGLWAYDGWNQLNYITEELRNPYRNLPLAIIIGIPLVTACYILMNVSYFTVMTATELLQSQAVAVTFGDRVLYPASWIVPLFVAFSTIGAANGTCFTAGRLIYVAGREGHMLKVLSYISVRRLTPAPAIIFYGIIATIYIIPGDINSLVNYFSFAAWLFYGLTILGLIVMRFTRKELERPIKVPVVIPVLMTLISVFLVLAPIISKPTWEYLYCVLFILSGLLFYFLFVHYKFGWAQKISKPITMHLQMLMEVVPPEEDPE

SEQ ID NO: 131 - protein, 1286 aa, Artificial Sequence
  ATP-binding cassette sub-family B (MDR) member 4 or Phosphatidylcholine translocator ABCB4 UniProt record number: P21439
MDLEAAKNGTAWRPTSAEGDFELGISSKQKRKKTKTVKMIGVLTLFRYSDWQDKLFMSLGTIMAIAHGSGLPLMMIVFGEMTDKFVDTAGNFSFPVNFSLSLLNPGKILEEEMTRYAYYYSGLGAGVLVAAYIQVSFWTLAAGRQIRKIRQKFFHAILRQEIGWFDINDTTELNTRLTDDISKISEGIGDKVGMFFQAVATFFAGFIVGFIRGWKLTLVIMAISPILGLSAAVWAKILSAFSDKELAAYAKAGAVAEEALGAIRTVIAFGGQNKELERYQKHLENAKEIGIKKAISANISMGIAFLLIYASYALAFWYGSTLVISKEYTIGNAMTVFFSILIGAFSVGQAAPCIDAFANARGAAYVIFDIIDNNPKIDSFSERGHKPDSIKGNLEFNDVHFSYPSRANVKILKGLNLKVQSGQTVALVGSSGCGKSTTVQLIQRLYDPDEGTINIDGQDIRNFNVNYLREIIGVVSQEPVLFSTTIAENICYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTVRNADVIAGFEDGVIVEQGSHSELMKKEGVYFKLVNMQTSGSQIQSEEFELNDEKAATRMAPNGWKSRLFRHSTQKNLKNSQMCQKSLDVETDGLEANVPPVSFLKVLKLNKTEWPYFVVGTVCAIANGGLQPAFSVIFSEIIAIFGPGDDAVKQQKCNIFSLIFLFLGIISFFTFFLQGFTFGKAGEILTRRLRSMAFKAMLRQDMSWFDDHKNSTGALSTRLATDAAQVQGATGTRLALIAQNIANLGTGIIISFIYGWQLTLLLLAVVPIIAVSGIVEMKLLAGNAKRDKKELEAAGKIATEAIENIRTVVSLTQERKFESMYVEKLYGPYRNSVQKAHIYGITFSISQAFMYFSYAGCFRFGAYLIVNGHMRFRDVILVFSAIVFGAVALGHASSFAPDYAKAKLSAAHLFMLFERQPLIDSYSEEGLKPDKFEGNITFNEVVFNYPTRANVPVLQGLSLEVKKGQTLALVGSSGCGKSTVVQLLERFYDPLAGTVFVDFGFQLLDGQEAKKLNVQWLRAQLGIVSQEPILFDCSIAENIAYGDNSRVVSQDEIVSAAKAANIHPFIETLPHKYETRVGDKGTQLSGGQKQRIAIARALIRQPQILLLDEATSALDTESEKVVQEALDKAREGRTCIVIAHRLSTIQNADLIVVFQNGRVKEHGTHQQLLAQKGIYFSMVSVQAGTQNL

SEQ ID NO: 132 - protein, 1382 aa, Artificial Sequence
  ATP-binding cassette sub-family C member 11 UniProt record number: Q96J66
MTRKRTYWVPNSSGGLVNRGIDIGDDMVSGLIYKTYTLQDGPWSQQERNPEAPGRAAVPPWGKYDAALRTMIPFRPKPRFPAPQPLDNAGLFSYLTVSWLTPLMIQSLRSRLDENTIPPLSVHDASDKNVQRLHRLWEEEVSRRGIEKASVLLVMLRFQRTRLIFDALLGICFCIASVLGPILIIPKILEYSEEQLGNVVHGVGLCFALFLSECVKSLSFSSSWIINQRTAIRFRAAVSSFAFEKLIQFKSVIHITSGEAISFFTGDVNYLFEGVCYGPLVLITCASLVICSISSYFIIGYTAFIAILCYLLVFPLAVFMTRMAVKAQHHTSEVSDQRIRVTSEVLTCIKLIKMYTWEKPFAKIIEDLRRKERKLLEKCGLVQSLTSITLFIIPTVATAVWVLIHTSLKLKLTASMAFSMLASLNLLRLSVFFVPIAVKGLTNSKSAVMRFKKFFLQESPVFYVQTLQDPSKALVFEEATLSWQQTCPGIVNGALELERNGHASEGMTRPRDALGPEEEGNSLGPELHKINLVVSKGMMLGVCGNTGSGKSSLLSAILEEMHLLEGSVGVQGSLAYVPQQAWIVSGNIRENILMGGAYDKARYLQVLHCCSLNRDLELLPFGDMTEIGERGLNLSGGQKQRISLARAVYSDRQIYLLDDPLSAVDAHVGKHIFEECIKKTLRGKTVVLVTHQLQYLEFCGQIILLENGKICENGTHSELMQKKGKYAQLIQKMHKEATSDMLQDTAKIAEKPKVESQALATSLEESLNGNAVPEHQLTQEEEMEEGSLSWRVYHHYIQAAGGYMVSCIIFFFVVLIVFLTIFSFWWLSYWLEQGSGTNSSRESNGTMADLGNIADNPQLSFYQLVYGLNALLLICVGVCSSGIFTKVTRKASTALHNKLFNKVFRCPMSFFDTIPIGRLLNCFAGDLEQLDQLLPIFSEQFLVLSLMVIAVLLIVSVLSPYILLMGAIIMVICFIYYMMFKKAIGVFKRLENYSRSPLFSHILNSLQGLSSIHVYGKTEDFISQFKRLTDAQNNYLLLFLSSTRWMALRLEIMTNLVTLAVALFVAFGISSTPYSFKVMAVNIVLQLASSFQATARIGLETEAQFTAVERILQYMKMCVSEAPLHMEGTSCPQGWPQHGEIIFQDYHMKYRDNTPTVLHGINLTIRGHEVVGIVGRTGSGKSSLGMALFRLVEPMAGRILIDGVDICSIGLEDLRSKLSVIPQDPVLLSGTIRFNLDPFDRHTDQQIWDALERTFLTKAISKFPKKLHTDVVENGGNFSVGERQLLCIARAVLRNSKIILIDEATASIDMETDTLIQRTIREAFQGCTVLVIAHRVTTVLNCDHILVMGNGKVVEFDRPEVLRKKPGSLFAALMATATSSLR

SEQ ID NO: 133 - protein, 845 aa, Artificial Sequence
  ATP-binding cassette sub-family F member 1 UniProt record number: Q8NE71
MPKAPKQQPPEPEWIGDGESTSPSDKVVKKGKKDKKIKKTFFEELAVEDKQAGEEEKVLKEKEQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKPQNKFAALDNEEEDKEEEIIKEKEPPKQGKEKAKKAEQGSEEEGEGEEEEEEGGESKADDPYAHLSKKEKKKLKKQMEYERQVASLKAANAAENDFSVSQAEMSSRQAMLENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 134 - protein, 691 aa, Artificial Sequence
  Solute carrier family 28 member 3 UniProt record number: Q9HAS3
MELRSTAAPRAEGYSNVGFQNEENFLENENTSGNNSIRSRAVQSREHTNTKQDEEQVTVEQDSPRNREHMEDDDEEMQQKGCLERRYDTVCGFCRKHKTTLRHIIWGILLAGYLVMVISACVLNFHRALPLFVITVAAIFFVVWDHLMAKYEHRIDEMLSPGRRLLNSHWFWLKWVIWSSLVLAVIFWLAFDTAKLGQQQLVSFGGLIMYIVLLFLFSKYPTRVYWRPVLWGIGLQFLLGLLILRTDPGFIAFDWLGRQVQTFLEYTDAGASFVFGEKYKDHFFAFKVLPIVVFFSTVMSMLYYLGLMQWIIRKVGWIMLVTTGSSPIESVVASGNIFVGQTESPLLVRPYLPYITKSELHAIMTAGFSTIAGSVLGAYISFGVPSSHLLTASVMSAPASLAAAKLFWPETEKPKITLKNAMKMESGDSGNLLEAATQGASSSISLVANIAVNLIAFLALLSFMNSALSWFGNMFDYPQLSFELICSYIFMPFSFMMGVEWQDSFMVARLIGYKTFFNEFVAYEHLSKWIHLRKEGGPKFVNGVQQYISIRSEIIATYALCGFANIGSLGIVIGGLTSMAPSRKRDIASGAVRALIAGTVACFMTACIAGILSSTPVDINCHHVLENAFNSTFPGNTTKVIACCQSLLSSTVAKGPGEVIPGGNHSLYSLKGCCTLLNPSTFNCNGISNTF

SEQ ID NO: 135 - protein, 842 aa, Artificial Sequence
  ATP-binding cassette sub-family B member 6, mito. UniProt record number: Q9NP58
MVTVGNYCEAEGPVGPAWMQDGLSPCFFFTLVPSTRMALGTLALVLALPCRRRERPAGADSLSWGAGPRISPYVLQLLLATLQAALPLAGLAGRVGTARGAPLPSYLLLASVLESLAGACGLWLLVVERSQARQRLAMGIWIKFRHSPGLLLLWTVAFAAENLALVSWNSPQWWWARADLGQQVQFSLWVLRYVVSGGLFVLGLWAPGLRPQSYTLQVHEEDQDVERSQVRSAAQQSTWRDFGRKLRLLSGYLWPRGSPALQLVVLICLGLMGLERALNVLVPIFYRNIVNLLTEKAPWNSLAWTVTSYVFLKFLQGGGTGSTGFVSNLRTFLWIRVQQFTSRRVELLIFSHLHELSLRWHLGRRTGEVLRIADRGTSSVTGLLSYLVFNVIPTLADIIIGIIYFSMFFNAWFGLIVFLCMSLYLTLTIVVTEWRTKFRRAMNTQENATRARAVDSLLNFETVKYYNAESYEVERYREAIIKYQGLEWKSSASLVLLNQTQNLVIGLGLLAGSLLCAYFVTEQKLQVGDYVLFGTYIIQLYMPLNWFGTYYRMIQTNFIDMENMFDLLKEETEVKDLPGAGPLRFQKGRIEFENVHFSYADGRETLQDVSFTVMPGQTLALVGPSGAGKSTILRLLFRFYDISSGCIRIDGQDISQVTQASLRSHIGVVPQDTVLFNDTIADNIRYGRVTAGNDEVEAAAQAAGIHDAIMAFPEGYRTQVGERGLKLSGGEKQRVAIARTILKAPGIILLDEATSALDTSNERAIQASLAKVCANRTTIVVAHRLSTVVNADQILVIKDGCIVERGRHEALLSRGGVYADMWQLQQGQEETSEDTKPQTMER

(19) United States
(12) Patent Application Publication
(10) Pub. No.: US 2022/0347309 A1
(43) Pub. Date: Nov. 3, 2022

(54) PYRROLOBENZODIAZEPINE RESISTANCE

(71) Applicants: ADC THERAPEUTICS SA, Epalinges (CH); MEDIMMUNE LIMITED, Cambridge (GB)

(72) Inventor: John Hartley, London (GB)

(21) Appl. No.: 17/414,622

(22) PCT Filed: Dec. 18, 2019
(86) PCT No.: PCT/EP2019/086079

§ 371 Date: Jun. 16, 2021

(30) Foreign Application Priority Data
Dec. 19, 2018 (GB) 1820725.8
Mar. 28, 2019 (GB) 1904342.1

(51) Int. Cl. A61K 47/68; A61K 31/5517; C07K 16/28; A61P 35/00; C12Q 1/6886

(52) U.S. Cl. CPC A61K 47/6803; A61K 31/5517; A61K 47/6849; C07K 16/28; A61P 35/00; C12Q 1/6886; C12Q 2600/158; C12Q 2600/178

(57) ABSTRACT

The present disclosure relates to methods of determining if a proliferative disorder such as cancer is resistant to treatment with a pyrrolobenzodiazepine (PBD) agent, such as a therapeutic antibody-drug conjugate (ADC) comprising a PBD warhead conjugated to an antibody (PBD-ADC). The present disclosure also describes methods of selecting subjects suitable for treatment with a PBD agent, and methods of reducing the resistance of a proliferative disorder to a PBD agent.

### EARLIER APPLICATIONS

This application claims priority from United Kingdom application GB1820725.8, filed on 19 Dec. 2018 and United Kingdom application GB1904342.1, filed on 28 Mar. 2019. The disclosure of these two priority documents is incorporated by reference into the present application for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 246,692 Byte ASCII (Text) file named “007914310_ST25.TXT,” created on Jan. 21, 2022.

### FIELD

The present disclosure relates to methods of determining if a proliferative disorder such as cancer is resistant to treatment with a pyrrolobenzodiazepine (PBD) agent, such as a therapeutic antibody-drug conjugate (ADC) comprising a PBD warhead conjugated to an antibody (PBD-ADC). The present disclosure also describes methods of selecting subjects suitable for treatment with a PBD agent, and methods of reducing the resistance of a proliferative disorder to a PBD agent.

### BACKGROUND

### Pyrrolobenzodiazepines

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994); Antonow, D. and Thurston, D. E., Chem. Rev. 2011 111 (4), 2815-2864). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102) (Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

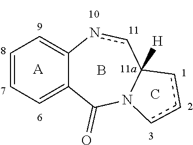

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

A particularly advantageous pyrrolobenzodiazepine compound is described by Gregson et al. (Chem. Commun. 1999, 797-798) as compound 1, and by Gregson et al. (J. Med. Chem. 2001, 44, 1161-1174) as compound 4a. This compound, also known as SG2000, is shown below:

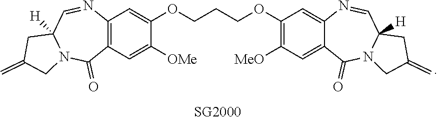

WO 2007/085930 describes the preparation of dimer PBD compounds having linker groups for connection to a cell binding agent, such as an antibody. The linker is present in the bridge linking the monomer PBD units of the dimer.

Dimer PBD compounds having linker groups for connection to a cell binding agent, such as an antibody, are described in WO2011/130613 and WO2011/130616. The linker in these compounds is attached to the PBD core via the C2 position, and are generally cleaved by action of an enzyme on the linker group. In WO 2011/130598, the linker in these compounds is attached to one of the available N10 positions on the PBD core, and are generally cleaved by action of an enzyme on the linker group.

Antibody-drug conjugates (ADCs) comprising PBD drug moieties (PBD-ADCs) and their therapeutic efficacy in treating a range of disorders are described in, for example, WO2014/057113, WO2014/057119, WO2016/166301, WO2016/166299, WO/2018/146189, and WO/2018/146199.

### Chemotherapy Resistance

Alongside surgery and radiotherapy, chemotherapy remains one of the most common treatments administered to subjects having proliferative disorders such as cancer. At its broadest, the term chemotherapy encompasses any method comprising the administration of chemical agents to a subject with the aim of treating the disorder; accordingly, it encompasses methods ranging from the use of nitrogen mustard in lymphosarcoma by Gilman and Goodman in the 1940's up to and including modern ADCs.

It was noted early in the chemotherapy field that responses to therapy were varied: some subjects respond well to a given therapy whilst others did not respond at all. Alternatively, some subjects were observed to respond well initially but then gradually stop responding and ultimately relapse, even when given increasing doses of the chemotherapeutic. These observations are now understood to be due to the ability of some cancers to resist, or develop resistance to, the cytotoxic effect of anticancer drugs.

Although numerous mechanisms have been associated with drug resistance in cancer, the number and complexity of these mechanisms and their interactions means a full understanding of how to overcome drug resistance is still a long way off. Mechanisms identified so far include increased drug efflux, drug inactivation and/or sequestration by enzymes, DNA repair, target modifications, and apoptosis defects (Ambudkar et al., An. R. Phar. & Tox. 1999; 39:361-398; Townsend D M. et al., Oncogene. 2003; 22(47):7369-7375; Eastman A., et al., Biochemistry, 1988; 27(13):4730-4734; Kavallaris M., et al., J. Clin. Invest. 1997; 100(5):1282-1293; Sethi T., Nature Med. 1999; 5(6):662-668). Additional contributing factors include ineffective drug delivery to the tumor, increased metabolism and thereforea shortened half-life of the drug, lack of drug specificity to the tumor, and tumor vasculature (Green S K., et al., Anticancer Drug Des. 1999; 14(2):153-168; Morin P J., Drug Resistance Updates. 2003; 6(4):169-172). These factors make it even harder to pinpoint the exact mechanism underlying resistance to a particular drug.

To further complicate our understanding of drug resistance, patients given chemotherapy gradually develop genetic mutations with each clonal expansion of the tumor cell. These mutations may result from either activation of proto-oncogenes or inactivation of tumor-suppressor genes. This continuous genomic instability eventually leads to tumor progression and metastatic changes, making treatment difficult in such patients; coexisting drug resistance of the tumors makes it even more difficult to treat the primary and metastatic lesions. Moreover, tumors that are resistant to one particular drug are either already cross-resistant or develop resistance to other chemotherapy drugs fairly quickly. Indeed, Small Cell Lung Cancer (SCLC), for example, typically initially responds to chemotherapy, but the patients invariably experience a relapse, and the tumor becomes resistant to chemotherapeutic treatment. As a result, drug resistance remains one of the main factors in the stubbornly poor 5-year survival rate (<5%) for SCLC (Shanker et al., Lung Cancer: Targets and Therapy 2010:1 23-36).

Understanding the mechanisms that contribute to resistance to various chemotherapeutics is therefore an important part of enabling the development of diagnostic tests capable of predicting resistance to those chemotherapeutics and so selecting the most effective treatment regime in any given clinical situation.

### SUMMARY

The present inventors have studied the mechanisms that enable tumour cells to resist the cytotoxic effects of PBD-agents, such as PBD-ADCs. Roles have been identified for a number of genes in PBD-resistance.

In particular, two members of the ABC transporter family, ABCC2 (multi-drug resistance protein 2, MRP2) and ABCG2 (breast cancer resistance protein, BCRP), were found to be significantly upregulated in the acquired resistant cell lines, and inhibitors to these two transporters recovered the cytotoxic sensitivity of the cells to the PBD-ADCs and the unconjugated PBD warhead. The administration of inhibitors was accompanied by the restoration of DNA interstrand cross-link (ICL) formation to levels seen in the parental (non-resistant) cell lines. Whilst this correlates with a reported role for ABCC2 and ABCG2 in some forms of chemotherapy resistance [Kathawala R, et al., Drug Resistance Updates 2015; 18, 1-17; Doyle L, et al., Oncogene 2003; 22(47), 7340-7358], no change was observed in other genes that had been more specifically linked to PBD resistance. For example, in Hartley J A, et al., Scientific Reports 2018; 8(1), 10479, the present authors reported that the PBD warhead SG3199 was susceptible to multidrug resistance mechanisms when compared in human tumor cell lines expressing MDR1, with the inhibitor verapamil able to reverse the resistance. However, in this study ABCB1 (MDR1, P-glycoprotein 1) was not significantly upregulated in any of the resistant cell lines. In addition, whilst members of the SLC transporter family were also found to be upregulated in some of the acquired cell lines, the SLC transporter inhibitor Lovastatin did not reverse the resistance. Together, this indicates that suggests that ABCG2 and ABCC2 are the two genes most likely to play an important role in determining sensitivity to PBD dimers and PBD-containing ADCs.

Accordingly, provided herein is a method for determining whether a proliferative disease in a subject is resistant to treatment with a pyrrolobenzodiazepine (PBD) agent, the method comprising determining whether one or more PBD-resistance genes are overexpressed in a sample from the subject, wherein overexpression of the one or more PBD-resistance genes indicates that the proliferative disease is resistant to treatment with the PBD agent.

Also provided herein is a method for selecting a subject for treatment with a pyrrolobenzodiazepine (PBD) agent, the method comprising the steps of, (a) determining whether one or more PBD-resistance genes are overexpressed in a sample from the subject, and (b) selecting the subject for treatment with the PBD agent if overexpression of the one or more PBD-resistance genes is not detected in the sample.

The one or more PBD-resistance genes are preferably selected from the group consisting of: ABCG2, ABCC2, SLCO2B1, SLC7A7, SLC22A3, SLCO2A1, ABCC12, ATP7A, AQP7, SLC5A1, SLC16A2, SLC7A9, ABCB4, ABCC11, ABCF1, SLC28A3, and ABCB6. The genes ABCG2, ABCC2, SLCO2B1, SLC7A7, and SLC22A3 are particularly preferred, with ABCG2 and ABCC2 even more particularly preferred, and with ABCG2 the most preferred.

Overexpression of a PBD-resistance gene may be determined by measuring the level of mRNA transcription from the one or more PBD-resistance genes, measuring the level of PBD-resistance polypeptide expression, or measuring PBD-resistance polypeptide activity. Overexpression of a gene is indicated when expression in a test sample exceeds a selected threshold relative to a control sample. In some cases overexpression of a PBD-resistance gene is indicated by an at least 2-fold increase relative to a control sample. In some cases, overexpression of a PBD-resistance gene is indicated by an increase relative to a control sample that has a p-value no greater than 0.05.

Test samples maybe take from a tumour sample or a circulating fluid such as blood, plasma, serum or lymph. The control may be the expression level in a healthy sample from the test subject, a sample from a healthy subject, a tumour sample from a disorder known not to be PBD-resistant, or a reference value from a control population. Preferably the test sample and control are taken from the same tissue.

The proliferative disease may be a benign, pre malignant, or malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis.

The PBD agent may be a PBD-ADC as described herein. Preferred PBD agents include ADCx25, ADCx19, ADCx22, ADCxPSMA, ADCxAXL, ADCxDLK1, ADCxKAAG1, and ADCxMesothelin.

The present disclosure also provides a method of reducing the resistance of a proliferative cell to a PBD-agent, the method comprising contacting the proliferative cell with an antagonist of one or more PBD-resistance genes.

Also disclosed herein is a method of treating a proliferative disease in a subject, wherein the proliferative disease is resistant to a PBD agent, the method comprising administering to the subject an antagonist of one or more PBD-resistance genes in combination with a therapeutically effective amount of the PBD agent.

The antagonist is administered before the PBD agent, simultaneous with the PBD agent, or after the PBD agent.

In some cases the antagonist reduces the level of mRNA transcription from the one or more PBD-resistance genes. In some cases the antagonist reduces the level of one or more PBD-resistance polypeptide expression. In some cases reduces the activity of one or more PBD-resistance polypeptide. Examples of suitable antagonists include small molecules that inhibit PBD-resistance gene (and/or polypeptide) activity, anti-PBD-resistance antibodies, and RNA agents that reduces the expression of one or more PBD-resistance gene.

Examples of suitable antagonists include small molecule inhibitors of PBD-resistance polypeptides; for example, small molecule inhibitors of ABC transporter proteins. In some case the small molecule inhibitor is selected from the group consisting of MK-571, Biricodar, Probenecid, Reversan, Fumitremorgin C, and Ko143.

Examples of antagonists that decrease PBD-resistance polypeptide expression, include antagonists that reduce ABCC2 expression such as miR-297 or miR-379. Examples of antagonists that decrease PBD-resistance polypeptide expression include antagonists that reduce ABCG2 expression, such as miR-200c, miR-212, miR-328, miR-519c, or miR-520h.

A further method disclosed herein describes a method of treating a proliferative disease in a subject, the method comprising a) selecting a subject for treatment with a PBD agent according to the methods described herein, and b) administering to the subject a therapeutically effective amount of the PBD agent, optionally wherein the PBD agent is administered according to the methods described herein.

### DETAILED DESCRIPTION

### PBD Resistance

The present disclosure concerns proliferative disorders which are resistant to treatment with a PBD agent, also referred to herein as “PBD-resistant proliferative disorders”, “PBD-resistant disorders”, “PBD-resistant cancers” and related terms.

A PBD-resistant proliferative disorder as described herein is a disorder characterized by the presence of a proliferative cell, or cell population such as a tumour, that is “resistant to treatment” with a PBD-agent. Such PBD-resistant cells exhibit a significantly different cellular or biological response to a PBD-agent than non-PBD-resistant control cells. For example, as compared to a non-PBD-resistant cell population, a PBD-resistant cell population exhibits a significantly higher rate of cell death or apoptosis, and/or a significantly lower rate of proliferation or growth, on treatment with a PBD-agent. Typically, the assessed cellular or biological response is the cytotoxicity of the PBD-agent; so, for example, a population of PDB-resistant breast cancer cells exhibits a significantly lower level of cytotoxicity than a non-PBD-resistant breast cancer cell population when the populations are exposed to the same concentration of a PBD-agent.

In some embodiments, a proliferative disorder is considered PBD-resistant if it is characterized by proliferative cells with a PBD agent IC₅₀ at least 2-fold greater than non-PBD-resistant control cells as described herein. In some embodiments the PBD agent IC₅₀ is at least 3-fold, at least 4-fold, at least 5-fold, at least 7-fold, at least 10-fold, at least 20-fold, or at least 50-fold greater than for non-PBD-resistant control cells. In some embodiments, the PBD agent assessed for resistance is RelE as described herein. In some embodiments, the PBD agent assessed for resistance is ADCx25, ADCx19, ADCx22, ADCxPSMA, ADCTxAXL, ADCTxDLK1, ADCxKAAG1, or ADCxMesothelin as described herein.

### PBD-Resistance Genes

| ABCG2 | |
|---|---|
| Full name: | ATP-binding cassette |
| | sub-family G member 2 |
| UniProt record number: | Q9UNQ0 |
| Genbank mRNA sequence ref: | AF103796.1 |
| Uniprot protein sequence ref: | Q9UNQ0-1 |
| Uniprot protein sequence: | |
| MSSSNVEVFIPVSQGNTNGFPATASNDLKAFTEGA | |
| VLSFHNICYRVKLKSGFLPCRKPVEKEILSNINGI | |
| MKPGLNAILGPTGGGKSSLLDVLAARKDPSGLSGD | |
| VLINGAPRPANFKCNSGYVVQDDVVMGTLTVRENL | |
| QFSAALRLATTMTNHEKNERINRVIQELGLDKVAD | |
| SKVGTQFIRGVSGGERKRTSIGMELITDPSILFLD | |
| EPTTGLDSSTANAVLLLLKRMSKQGRTIIFSIHQP | |
| RYSIFKLFDSLTLLASGRLMFHGPAQEALGYFESA | |
| GYHCEAYNNPADFFLDIINGDSTAVALNREEDFKA | |
| TEIIEPSKQDKPLIEKLAEIYVNSSFYKETKAELH | |
| QLSGGEKKKKITVFKEISYTTSFCHQLRWVSKRSF | |
| KNLLGNPQASIAQIIVTVVLGLVIGAIYFGLKNDS | |
| TGIQNRAGVLFFLTTNQCFSSVSAVELFVVEKKLF | |
| IHEYISGYYRVSSYFLGKLLSDLLPMRMLPSIIFT | |
| CIVYFMLGLKPKADAFFVMMFTLMMVAYSASSMAL | |
| AIAAGQSVVSVATLLMTICFVFMMIFSGLLVNLTT | |
| IASWLSWLQYFSIPRYGFTALQHNEFLGQNFCPGL | |
| NATGNNPCNYATCTGEEYLVKQGIDLSPWGLWKNH | |
| VALACMIVIFLTIAYLKLLFLKKYS | |
| ABCC2 | |
| Full name: | ATP-binding cassette |
| | sub-family C (MRP) member 2 |
| | or Canalicular multispecific |
| | organic anion transporter 1 |
| UniProt record number: | Q92887 |
| Genbank mRNA sequence ref: | U63970.1 |
| Uniprot protein sequence ref: | Q92887-1 |
| Uniprot protein sequence: | |
| MLEKFCNSTFWNSSFLDSPEADLPLCFEQTVLVWI | |
| PLGYLWLLAPWQLLHVYKSRTKRSSTTKLYLAKQV | |
| FVGFLLILAAIELALVLTEDSGQATVPAVRYTNPS | |
| LYLGTWLLVLLIQYSRQWCVQKNSWFLSLFWILSI | |
| LCGTFQFQTLIRTLLQGDNSNLAYSCLFFISYGFQ | |
| ILILIFSAFSENNESSNNPSSIASFLSSITYSWYD | |
| SIILKGYKRPLTLEDVWEVDEEMKTKTLVSKFETH | |
| MKRELQKARRALQRRQEKSSQQNSGARLPGLNKNQ | |
| SQSQDALVLEDVEKKKKKSGTKKDVPKSWLMKALF | |
| KTFYMVLLKSFLLKLVNDIFTFVSPQLLKLLISFA | |
| SDRDTYLWIGYLCAILLFTAALIQSFCLQCYFQLC | |
| FKLGVKVRTAIMASVYKKALTLSNLARKEYTVGET | |
| VNLMSVDAQKLMDVTNFMHMLWSSVLQIVLSIFFL | |
| WRELGPSVLAGVGVMVLVIPINAILSTKSKTIQVK | |
| NMKNKDKRLKIMNEILSGIKILKYFAWEPSFRDQV | |
| QNLRKKELKNLLAFSQLQCVVIFVFQLTPVLVSVV | |
| TFSVYVLVDSNNILDAQKAFTSITLFNILRFPLSM | |
| LPMMISSMLQASVSTERLEKYLGGDDLDTSAIRHD | |
| CNFDKAMQFSEASFTWEHDSEATVRDVNLDIMAGQ | |
| LVAVIGPVGSGKSSLISAMLGEMENVHGHITIKGT | |
| TAYVPQQSWIQNGTIKDNILFGTEFNEKRYQQVLE | |
| ACALLPDLEMLPGGDLAEIGEKGINLSGGQKQRIS | |
| LARATYQNLDIYLLDDPLSAVDAHVGKHIFNKVLG | |
| PNGLLKGKTRLLVTHSMHFLPQVDEIVVLGNGTIV | |
| EKGSYSALLAKKGEFAKNLKTFLRHTGPEEEATVH | |
| DGSEEEDDDYGLISSVEEIPEDAASITMRRENSFR | |
| RTLSRSSRSNGRHLKSLRNSLKTRNVNSLKEDEEL | |
| VKGQKLIKKEFIETGKVKFSIYLEYLQAIGLFSIF | |
| FIILAFVMNSVAFIGSNLWLSAWTSDSKIFNSTDY | |
| PASQRDMRVGVYGALGLAQGIFVFIAHFWSAFGFV | |
| HASNILHKQLLNNILRAPMRFFDTTPTGRIVNRFA | |
| GDISTVDDTLPQSLRSWITCFLGIISTLVMICMAT | |
| PVFTIIVIPLGIIYVSVQMFYVSTSRQLRRLDSVT | |
| RSPIYSHFSETVSGLPVIRAFEHQQRFLKHNEVRI | |
| DTNQKCVFSWITSNRWLAIRLELVGNLTVFFSALM | |
| MVIYRDTLSGDTVGFVLSNALNITQTLNWLVRMTS | |
| EIETNIVAVERITEYTKVENEAPWVTDKRPPPDWP | |
| SKGKIQFNNYQVRYRPELDLVLRGITCDIGSMEKI | |
| GVVGRTGAGKSSLTNCLFRILEAAGGQIIIDGVDI | |
| ASIGLHDLREKLTIIPQDPILFSGSLRMNLDPFNN | |
| YSDEEIWKALELAHLKSFVASLQLGLSHEVTEAGG | |
| NLSIGQRQLLCLGRALLRKSKILVLDEATAAVDLE | |
| TDNLIQTTIQNEFAHCTVITIAHRLHTIMDSDKVM | |
| VLDNGKIIECGSPEELLQIPGPFYFMAKEAGIENV | |
| NSTKF | |
| SLCO2B1 | |
| Full name: | Solute carrier organic |
| | anion transporter fam. mem. 2B1 |
| UniProt record number: | O94956 |
| Genbank mRNA sequence ref: | AB026256.1 |
| Uniprot protein sequence ref: | O94956-1 |
| Uniprot protein sequence: | |
| MGPRIGPAGEVPQVPDKETKATMGTENTPGGKASP | |
| DPQDVRPSVFHNIKLFVLCHSLLQLAQLMISGYLK | |
| SSISTVEKRFGLSSQTSGLLASFNEVGNTALIVFV | |
| SYFGSRVHRPRMIGYGAILVALAGLLMTLPHFISE | |
| PYRYDNTSPEDMPQDFKASLCLPTTSAPASAPSNG | |
| NCSSYTETQHLSVVGIMFVAQTLLGVGGVPIQPFG | |
| ISYIDDFAHNSNSPLYLGILFAVTMMGPGLAFGLG | |
| SLMLRLYVDINQMPEGGISLTIKDPRWVGAWWLGF | |
| LIAAGAVALAAIPYFFFPKEMPKEKRELQFRRKVL | |
| AVTDSPARKGKDSPSKQSPGESTKKQDGLVQIAPN | |
| LTVIQFIKVFPRVLLQTLRHPIFLLVVLSQVCLSS | |
| MAAGMAIFLPKFLERQFSITASYANLLIGCLSFPS | |
| VIVGIVVGGVLVKRLHLGPVGCGALCLLGMLLCLF | |
| FSLPLFFIGCSSHQIAGITHQTSAHPGLELSPSCM | |
| EACSCPLDGFNPVCDPSTRVEYITPCHAGCSSWVV | |
| QDALDNSQVFYTNCSCVVEGNPVLAGSCDSTCSHL | |
| VVPFLLLVSLGSALACLTHTPSFMLILRGVKKEDK | |
| TLAVGIQFMFLRILAWMPSPVIHGSAIDTTCVHWA | |
| LSCGRRAVCRYYNNDLLRNRFIGLQFFFKTGSVIC | |
| FALVLAVLRQQDKEARTKESRSSPAVEQQLLVSGP | |
| GKKPEDSRV | |
| SLC7A7 | |
| Full name: | Solute carrier family 7, number 7 |
| | or Y + L amino acid transporter 1 |
| UniProt record number: | Q9UM01 |
| Genbank mRNA sequence ref: | AF092032.1 |
| Uniprot protein sequence ref: | Q9UM01-1 |
| Uniprot protein sequence: | |
| MVDSTEYEVASQPEVETSPLGDGASPGPEQVKLKK | |
| EISLLNGVCLIVGNMIGSGIFVSPKGVLIYSASFG | |
| LSLVIWAVGGLFSVFGALCYAELGTTIKKSGASYA | |
| YILEAFGGFLAFIRLWTSLLIIEPTSQAIIAITFA | |
| NYMVQPLFPSCFAPYAASRLLAAACICLLTFINCA | |
| YVKWGTLVQDIFTYAKVLALIAVIVAGIVRLGQGA | |
| STHFENSFEGSSFAVGDIALALYSALFSYSGWDTL | |
| NYVTEEIKNPERNLPLSIGISMPIVTIIYILTNVA | |
| YYTVLDMRDILASDAVAVTFADQIFGIFNWIIPLS | |
| VALSCFGGLNASIVAASRLFFVGSREGHLPDAICM | |
| IHVERFTPVPSLLFNGIMALIYLCVEDIFQLINYY | |
| SFSYWFFVGLSIVGQLYLRWKEPDRPRPLKLSVFF | |
| PIVFCLCTIFLVAVPLYSDTINSLIGIAIALSGLP | |
| FYFLIIRVPEHKRPLYLRRIVGSATRYLQVLCMSV | |
| AAEMDLEDGGEMPKQRDPKSN | |
| SLC22A3 | |
| Full name: | Solute carrier family 22 member 3 |
| UniProt record number: | O75751 |
| Genbank mRNA sequence ref: | AJ001417.1 |
| Uniprot protein sequence ref: | O75751-1 |
| Uniprot protein sequence: | |
| MPSFDEALQRVGEFGRFQRRVFLLLCLTGVTFAFLF | |
| VGVVFLGTQPDHYWCRGPSAAALAERCGWSPEEEW | |
| NRTAPASRGPEPPERRGRCQRYLLEAANDSASATS | |
| ALSCADPLAAFPNRSAPLVPCRGGWRYAQAHSTIV | |
| SEFDLVCVNAWMLDLTQAILNLGFLTGAFTLGYAA | |
| DRYGRIVIYLLSCLGVGVTGVVVAFAPNFPVFVIF | |
| RFLQGVFGKGTWMTCYVIVTEIVGSKQRRIVGIVI | |
| QMFFTLGIIILPGIAYFIPNWQGIQLAITLPSFLF | |
| LLYYWVVPESPRWLITRKKGDKALQILRRIAKCNG | |
| KYLSSNYSEITVTDEEVSNPSFLDLVRTPQMRKCT | |
| LILMFAWFTSAVVYQGLVMRLGIIGGNLYIDFFIS | |
| GVVELPGALLILLTIERLGRRLPFAASNIVAGVAC | |
| LVTAFLPEGIAWLRTTVATLGRLGITMAFEIVYLV | |
| NSELYPTTLRNFGVSLCSGLCDFGGIIAPFLLFRL | |
| AAVWLELPLIIFGILASICGGLVMLLPETKGIALP | |
| ETVDDVEKLGSPHSCKCGRNKKTPVSRSHL | |
| SLCO2A1 | |
| Full name: | Solute carrier organic anion |
| | transporter fam. mem. 2A1 |
| UniProt record number: | Q92959 |
| Genbank mRNA sequence ref: | U70867.1 |
| Uniprot protein sequence ref: | Q92959-1 |
| Uniprot protein sequence: | |
| MGLLPKLGASQGSDTSTSRAGRCARSVFGNIKVFV | |
| LCQGLLQLCQLLYSAYFKSSLTTIEKRFGLSSSSS | |
| GLISSLNEISNAILIIFVSYFGSRVHRPRLIGIGG | |
| LFLAAGAFILTLPHFLSEPYQYTLASTGNNSRLQA | |
| ELCQKHWQDLPPSKCHSTTQNPQKETSSMWGLMVV | |
| AQLLAGIGTVPIQPFGISYVDDFSEPSNSPLYISI | |
| LFAISVFGPAFGYLLGSVMLQIFVDYGRVNTAAVN | |
| LVPGDPRWIGAWWLGLLISSALLVLTSFPFFFFPR | |
| AMPIGAKRAPATADEARKLEEAKSRGSLVDFIKRF | |
| PCIFLRLLMNSLFVLVVLAQCTFSSVIAGLSTFLN | |
| KFLEKQYGTSAAYANFLIGAVNLPAAALGMLFGGI | |
| LMKRFVFSLQAIPRIATTIITISMILCVPLFFMGC | |
| STPTVAEVYPPSTSSSIHPQSPACRRDCSCPDSIF | |
| HPVCGDNGIEYLSPCHAGCSNINMSSATSKQLIYL | |
| NCSCVTGGSASAKTGSCPVPCAHFLLPAIFLISFV | |
| SLIACISHNPLYMMVLRVVNQEEKSFAIGVQFLLM | |
| RLLAWLPSPALYGLTIDHSCIRWNSLCLGRRGACA | |
| YYDNDALRDRYLGLQMGYKALGMLLLCFISWRVKK | |
| NKEYNVQKAAGLI | |
| ABCC12 | |
| Full name: | ATP-binding cassette |
| | sub-family C (MRP) member 12 |
| | or Multidrug resistance- |
| | associated protein 9 |
| UniProt record number: | Q96J65 |
| Genbank mRNA sequence ref: | AF395908.1 |
| Uniprot protein sequence ref: | Q96J65-1 |
| Uniprot protein sequence: | |
| MVGEGPYLISDLDQRGRRRSFAERYDPSLKTMIPV | |
| RPCARLAPNPVDDAGLLSFATFSWLTPVMVKGYRQ | |
| RLTVDTLPPLSTYDSSDTNAKRFRVLWDEEVARVG | |
| PEKASLSHVVWKFQRTRVLMDIVANILCIIMAAIG | |
| PVILIHQILQQTERTSGKVWVGIGLCIALFATEF | |
| TKVFFWALAWAINYRTAIRLKVALSTLVFENLVSF | |
| KTLTHISVGEVLNILSSDSYSLFEAALFCPLPATI | |
| PILMVFCAAYAFFILGPTALIGISVYVIFIPVQMF | |
| MAKLNSAFRRSAILVTDKRVQTMNEFLTCIRLIKM | |
| YAWEKSFTNTIQDIRRRERKLLEKAGFVQSGNSAL | |
| APIVSTIAIVLTLSCHILLRRKLTAPVAFSVIAMF | |
| NVMKFSIAILPFSIKAMAEANVSLRRMKKILIDKS | |
| PPSYITQPEDPDTVLLLANATLTWEHEASRKSTPK | |
| KLQNQKRHLCKKQRSEAYSERSPPAKGATGPEEQS | |
| DSLKSVLHSISFVVRKGKILGICGNVGSGKSSLLA | |
| ALLGQMQLQKGVVAVNGTLAYVSQQAWIFHGNVRE | |
| NILFGEKYDHQRYQHTVRVCGLQKDLSNLPYGDLT | |
| EIGERGLNLSGGQRQRISLARAVYSDRQLYLLDDP | |
| LSAVDAHVGKHVFEECIKKTLRGKTVVLVTHQLQF | |
| LESCDEVILLEDGEICEKGTHKELMEERGRYAKLI | |
| HNLRGLQFKDPEHLYNAAMVEAFKESPAEREEDAG | |
| IIVLAPGNEKDEGKESETGSEFVDTKVPEHQLIQT | |
| ESPQEGTVTWKTYHTYIKASGGYLLSLFTVFLFLL | |
| MIGSAAFSNWWLGLWLDKGSRMTCGPQGNRTMCEV | |
| GAVLADIGQHVYQWVYTASMVFMLVFGVTKGFVFT | |
| KTTLMASSSLHDTVFDKILKSPMSFFDTTPTGRLM | |
| NRFSKDMDELDVRLPFHAENFLQQFFMVVFILVIL | |
| AAVFPAVLLVVASLAVGFFILLRIFHRGVQELKKV | |
| ENVSRSPWFTHITSSMQGLGIIHAYGKKESCITYH | |
| LLYFNCALRWFALRMDVLMNILTFTVALLVTLSFS | |
| SISTSSKGLSLSYIIQLSGLLQVCVRTGTETQAKF | |
| TSVELLREYISTCV | |
| PECTHPLKVGTCPKDWPSRGEITFRDYQMRYRDNT | |
| PLVLDSLNLNIQSGQTVGIVGRTGSGKSSLGMALF | |
| RLVEPASGTIFIDEVDICILSLEDLRTKLTVIPQD | |
| PVLFVGTVRYNLDPFESHTDEMLWQVLERTFMRDT | |
| IMKLPEKLQAEVTENGENFSVGERQLLCVARALLR | |
| NSKIILLDEATASMDSKTDTLVQNTIKDAFKGCTV | |
| LTIAHRLNTVLNCDHVLVMENGKVIEFDKPEVLAE | |
| KPDSAFAMLLAAEVRL | |
| ATP7A | |
| Full name: | ATPase, Cu++ transporting |
| | alpha polypeptide |
| | or Copper- |
| | transporting ATPase 1 |
| UniProt record number: | Q04656 |
| Genbank mRNA sequence ref: | L06133.1 |
| Uniprot protein sequence ref: | Q04656-1 |
| Uniprot protein sequence: | |
| MDPSMGVNSVTISVEGMTCNSCVWTIEQQIGKVNG | |
| VHHIKVSLEEKNATIIYDPKLQTPKTLQEAIDDMG | |
| FDAVIHNPDPLPVLTDTLFLTVTASLTLPVVDHIQ | |
| STLLKTKGVTDIKIYPQKRTVAVTIIPSIVNANQI | |
| KELVPELSLDTGTL | |
| EKKSGACEDHSMAQAGEVVLKMKVEGMTCHSCTST | |
| IEGKIGKLQGVQRIKVSLDNQEATIVYQPHLISVE | |
| EMKKQIEAMGFPAFVKKQPKYLKLGAIDVERLKNT | |
| PVKSSEGSQQRSPSYTNDSTATFIIDGMHCKSCVS | |
| NIESTLSALQYVSSIVVSLENRSAIVKYNASSVTP | |
| ESLRKAIEAVSPGLYRVSITSEVESTSNSPSSSSL | |
| QKIPLNWSQPLTQETVINIDGMTCNSCVQSIEGVI | |
| SKKPGVKSIRVSLANSNGTVEYDPLLTSPETLRGA | |
| IEDMGFDATLSDTNEPLVVIAQPSSEMPLLTSTNE | |
| FYTKGMTPVQDKEEGKNSSKCYIQVTGMTCASCVA | |
| NIERNLRREEGIYSILVALMAGKAEVRYNPAVIQP | |
| PMIAEFIRELGFGATVIENADEGDGVLELVVRGMT | |
| CASCVHKIESSLTKHRGILYCSVALATNKAHIKYD | |
| PEIIGPRDIIHTIESLGFEASLVKKDRSASHLDHK | |
| REIRQWRRSFLVSLFFCIPVMGLMIYMMVMDHHFA | |
| TLHHNQNMSKEEMINLHSSMFLERQILPGLSVMNL | |
| LSFLLCVPVQFFGGWYFYIQAYKALKHKTANMDVL | |
| IVLATTIAFAYSLIILLVAMYERAKVNPITFFDTP | |
| PMLFVFIALGRWLEHIAKGKTSEALAKLISLQATE | |
| ATIVTLDSDNILLSEEQVDVELVQRGDIIKVVPGG | |
| KFPVDGRVIEGHSMVDESLITGEAMPVAKKPGSTV | |
| IAGSINQNGSLLICATHVGADTTLSQIVKLVEEAQ | |
| TSKAPIQQFADKLSGYFVPFIVFVSIATLLVWIVI | |
| GFLNFEIVETYFPGYNRSISRTETIIRFAFQASIT | |
| VLCIACPCSLGLATPTAVMVGTGVGAQNGILIKGG | |
| EPLEMAHKVKVVVFDKTGTITHGTPVVNQVKVLTE | |
| SNRISHHKILAIVGTAESNSEHPLGTAITKYCKQE | |
| LDTETLGTCIDFQVVPGCGISCKVTNIEGLLHKNN | |
| WNIEDNNIKNASLVQIDASNEQSSTSSSMIIDAQI | |
| SNALNAQQYKVLIGNREWMIRNGLVINNDVNDFMT | |
| EHERKGRTAVLVAVDDELCGLIAIADTVKPEAELA | |
| IHILKSMGLEVVLMTGDNSKTARSIASQVGITKVF | |
| AEVLPSHKVAKVKQLQEEGKRVAMVGDGINDSPAL | |
| AMANVGIAIGTGTDVAIEAADVVLIRNDLLDVVAS | |
| IDLSRKTVKRIRINFVFALIYNLVGIPIAAGVFMP | |
| IGLVLQPWMGSAAMAASSVSVVLSSLFLKLYRKPT | |
| YESYELPARSQIGQKSPSEISVHVGIDDTSRNSPK | |
| LGLLDRIVNYSRASINSLLSDKRSLNSVVTSEPDK | |
| HSLLVGDFREDDDTAL | |
| AQP7 | |
| Full name: | Aquaporin-7 |
| UniProt record number: | O14520 |
| Genbank mRNA sequence ref: | AB006190.1 |
| Uniprot protein sequence ref: | O14520-1 |
| Uniprot protein sequence: | |
| MVQASGHRRSTRGSKMVSWSVIAKIQEILQRKMVR | |
| EFLAEFMSTYVMMVFGLGSVAHMVLNKKYGSYLGV | |
| NLGFGFGVTMGVHVAGRISGAHMNAAVTFANCALG | |
| RVPWRKFPVYVLGQFLGSFLAAATIYSLFYTAILH | |
| FSGGQLMVTGPVATAGIFATYLPDHMTLWRGFLNE | |
| AWLTGMLQLCLFAITDQENNPALPGTEALVIGILW | |
| IIGVSLGMNTGYAINPSRDLPPRIFTFIAGWGKQV | |
| FSNGENWWWVPVVAPLLGAYLGGIIYLVFIGSTIP | |
| REPLKLEDSVAYEDHGITVLPKMGSHEPTISPLTP | |
| VSVSPANRSSVHPAPPLHESMALEHF | |
| SLC5A1 | |
| Full name: | Solute carrier |
| | family 5 member 1 |
| | or Sodium/glucose |
| | cotransporter 1 |
| UniProt record number: | P13866 |
| Genbank mRNA sequence ref: | M24847.1 |
| Uniprot protein sequence ref: | P13866-1 |
| Uniprot protein sequence: | |
| MDSSTWSPKTTAVTRPVETHELIRNAADISIIVIY | |
| FVVVMAVGLWAMFSTNRGTVGGFFLAGRSMVWWPI | |
| GASLFASNIGSGHFVGLAGTGAASGIAIGGFEWNA | |
| LVLVVVLGWLFVPIYIKAGVVTMPEYLRKRFGGQR | |
| IQVYLSLLSLLLYIFTKISADIFSGAIFINLALGL | |
| NLYLAIFLLLAITALYTITGGLAAVIYTDTLQTVI | |
| MLVGSLILTGFAFHEVGGYDAFMEKYMKAIPTIVS | |
| DGNTTFQEKCYTPRADSFHIFRDPLTGDLPWPGFI | |
| FGMSILTLWYWCTDQVIVQRCLSAKNMSHVKGGCI | |
| LCGYLKLMPMFIMVMPGMISRILYTEKIACVVPSE | |
| CEKYCGTKVGCTNIAYPTLVVELMPNGLRGLMLSV | |
| MLASLMSSLTSIFNSASTLFTMDIYAKVRKRASEK | |
| ELMIAGRLFILVLIGISIAWVPIVQSAQSGQLFD | |
| YIQSITSYLGPPIAAVFLLAIFWKRVNEPGAFWGL | |
| ILGLLIGISRMITEFAYGTGSCMEPSNCPTIICGV | |
| HYLYFAIILFAISFITIVVISLLTKPIPDVHLYRL | |
| CWSLRNSKEERIDLDAEEENIQEGPKETIEIETQV | |
| PEKKKGIFRRAYDLFCGLEQHGAPKMTEEEEKAMK | |
| MKMTDTSEKPLWRTVLNVNGIILVTVAVFCHAYFA | |
| SLC16A2 | |
| Full name: | Solute carrier family |
| | 16 member 2 or |
| | Monocarboxylate |
| | transporter 8 |
| UniProt record number: | P36021 |
| Genbank mRNA sequence ref: | AH005330.2 |
| Uniprot protein sequence ref: | P36021-1 |
| Uniprot protein sequence: | |
| MALQSQASEEAKGPWQEADQEQQEPVGSPEPESEP | |
| EPEPEPEPVPVPPPEPQPEPQPLPDPAPLPELEFE | |
| SERVHEPEPTPTVETRGTARGFQPPEGGFGWVVVF | |
| AATWCNGSIFGIHNSVGILYSMLLEEEKEKNRQVE | |
| FQAAWVGALAMGMIFFCSPIVSIFTDRLGCRITAT | |
| AGAAVAFIGLHTSSFTSSLSLRYFTYGILFGCGCS | |
| FAFQPSLVILGHYFQRRLGLANGVVSAGSSIFSMS | |
| FPFLIRMLGDKIKLAQTFQVLSTFMFVLMLLSLTY | |
| RPLLPSSQDTPSKRGVRTLHQRFLAQLRKYFNMRV | |
| FRQRTYRIWAFGIAAAALGYFVPYVHLMKYVEEEF | |
| SEIKETWVLLVCIGATSGLGRLVSGHISDSIPGL | |
| KKIYLQVLSFLLLGLMSMMIPLCRDFGGLIVVCLF | |
| LGLCDGFFITIMAPIAFELVGPMQASQAIGYLLGM | |
| MALPMIAGPPIAGLLRNCFGDYHVAFYFAGVPPII | |
| GAVILFFVPLMHQRMFKKEQRDSSKDKMLAPDPDP | |
| NGELLPGSPNPEEPI | |
| SLC7A9 | |
| Full name: | Solute carrier family |
| | 7 member 9 |
| | or b(0, +)-type amino |
| | acid transporter 1 |
| UniProt record number: | P82251 |
| Genbank mRNA sequence ref: | AF141289.1 |
| Uniprot protein sequence ref: | P82251-1 |
| Uniprot protein sequence: | |
| MGDTGLRKRREDEKSIQSQEPKTTSLQKELGLISG | |
| ISIIVGTIIGSGIFVSPKSVLSNTEAVGPCLIIWA | |
| ACGVLATLGALCFAELGTMITKSGGEYPYLMEAYG | |
| PIPAYLFSWASLIVIKPTSFAIICLSFSEYVCAPF | |
| YVGCKPPQIVVKCLAAAAILFISTVNSLSVRLGSY | |
| VQNIFTAAKLVIVAIIIISGLVLLAQGNTKNFDNS | |
| FEGAQLSVGAISLAFYNGLWAYDGWNQLNYITEEL | |
| RNPYRNLPLAIIIGIPLVTACYILMNVSYFTVMTA | |
| TELLQSQAVAVTFGDRVLYPASWIVPLFVAFSTIG | |
| AANGTCFTAGRLIYVAGREGHMLKVLSYISVRRLT | |
| PAPAIIFYGIIATIYIIPGDINSLVNYFSFAAWLF | |
| YGLTILGLIVMRFTRKELERPIKVPVVIPVLMTLI | |
| SVFLVLAPIISKPTWEYLYCVLFILSGLLFYFLFV | |
| HYKFGWAQKISKPITMHLQMLMEVVPPEEDPE | |
| ABCB4 | |
| Full name: | ATP-binding cassette |
| | sub-family B (MDR) |
| | member 4 or |
| | Phosphatidylcholine |
| | translocator ABCB4 |
| UniProt record number: | P21439 |
| Genbank mRNA sequence ref: | M23234.1 |
| Uniprot protein sequence ref: | P21439-1 |
| Uniprot protein sequence: | |
| MDLEAAKNGTAWRPTSAEGDFELGISSKQKRKKTK | |
| TVKMIGVLTLFRYSDWQDKLFMSLGTIMAIAHGSG | |
| LPLMMIVFGEMTDKFVDTAGNFSFPVNFSLSLLNP | |
| GKILEEEMTRYAYYYSGLGAGVLVAAYIQVSFWTL | |
| AAGRQIRKIRQKFFHAILRQEIGWFDINDTTELNT | |
| RLTDDISKISEGIGDKVGMFFQAVATFFAGFIVGF | |
| IRGWKLTLVIMAISPILGLSAAVWAKILSAFSDKE | |
| LAAYAKAGAVAEEALGAIRTVIAFGGQNKELERYQ | |
| KHLENAKEIGIKKAISANISMGIAFLLIYASYALA | |
| FWYGSTLVISKEYTIGNAMTVFFSILIGAFSVGQA | |
| APCIDAFANARGAAYVIFDIIDNNPKIDSFSERGH | |
| KPDSIKGNLEFNDVHFSYPSRANVKILKGLNLKVQ | |
| SGQTVALVGSSGCGKSTTVQLIQRLYDPDEGTINI | |
| DGQDIRNFNVNYLREIIGVVSQEPVLFSTTIAENI | |
| CYGRGNVTMDEIKKAVKEANAYEFIMKLPQKFDTL | |
| VGERGAQLSGGQKQRIAIARALVRNPKILLLDEAT | |
| SALDTESEAEVQAALDKAREGRTTIVIAHRLSTVR | |
| NADVIAGFEDGVIVEQGSHSELMKKEGVYFKLVNM | |
| QTSGSQIQSEEFELNDEKAATRMAPNGWKSRLFRH | |
| STQKNLKNSQMCQKSLDVETDGLEANVPPVSFLKV | |
| LKLNKTEWPYRNGTVCAIANGGLQPAFSVIFSEII | |
| AIFGPGDDAVKQQKCNIFSLIFLFLGIISFFTFFL | |
| QGFTFGKAGEILTRRLRSMAFKAMLRQDMSWFDDH | |
| KNSTGALSTRLATDAAQVQGATGTRLALIAQNIAN | |
| LGTGIIISFIYGWQLTLLLLAVVPIIAVSGIVEMK | |
| LLAGNAKRDKKELEAAGKIATEAIENIRTVVSLTQ | |
| ERKFESMYVEKLYGPYRNSVQKAHIYGITFSISQA | |
| FMYFSYAGCFRFGAYLIVNGHMRFRDVILVFSAIV | |
| FGAVALGHASSFAPDYAKAKLSAAHLFMLFERQPL | |
| IDSYSEEGLKPDKFEGNITFNEVVFNYPTRANVPV | |
| LQGLSLEVKKGQTLALVGSSGCGKSTVVQLLERFY | |
| DPLAGTVFVDFGFQLLDGQEAKKLNVQWLRAQLGI | |
| VSQEPILFDCSIAENIAYGDNSRVVSQDEIVSAAK | |
| AANIHPFIETLPHKYETRVGDKGTQLSGGQKQRIA | |
| IARALIRQPQILLLDEATSALDTESEKVVQEALDK | |
| AREGRTCIVIAHRLSTIQNADLIVVFQNGRVKEHG | |
| THQQLLAQKGIYFSMVSVQAGTQNL | |
| ABCC11 | |
| Full name: | ATP-binding cassette |
| | sub-family C member 11 |
| UniProt record number: | Q96J66 |
| Genbank mRNA sequence ref: | AY040219.1 |
| Uniprot protein sequence ref: | Q96J66-1 |
| Uniprot protein sequence: | |
| MTRKRTYWVPNSSGGLVNRGIDIGDDMVSGLIYK | |
| TYTLQDGPWSQQERNPEAPGRAAVPPWGKYDAALR | |
| TMIPFRPKPRFPAPQPLDNAGLFSYLTVSWLTPLM | |
| IQSLRSRLDENTIPPLSVHDASDKNVQRLHRLWEE | |
| EVSRRGIEKASVLLVMLRFQRTRLIFDALLGICFC | |
| IASVLGPILIIPKILEYSEEQLGNVVHGVGLCFAL | |
| FLSECVKSLSFSSSWIINQRTAIRFRAAVSSFAFE | |
| KLIQFKSVIHITSGEAISFFTGDVNYLFEGVCYGP | |
| LVLITCASLVICSISSYFIIGYTAFIAILCYLLVF | |
| PLAVFMTRMAVKAQHHTSEVSDQRIRVTSEVLTCI | |
| KLIKMYTWEKPFAKIIEDLRRKERKLLEKCGLVQS | |
| LTSITLFIIPTVATAVWVLIHTSLKLKLTASMAF | |
| SMLASLNLLRLSVFFVPIAVKGLTNSKSAVMRFKK | |
| FFLQESPVFYVQTLQDPSKALVFEEATLSWQQTCP | |
| GIVNGALELERNGHASEGMTRPRDALGPEEEGNSL | |
| GPELHKINLVVSKGMMLGVCGNTGSGKSSLLSAIL | |
| EEMHLLEGSVGVQGSLAYVPQQAWIVSGNIRENIL | |
| MGGAYDKARYLQVLHCCSLNRDLELLPFGDMTEIG | |
| ERGLNLSGGQKQRISLARAVYSDRQIYLLDDPLSA | |
| VDAHVGKHIFEECIKKTLRGKTVVLVTHQLQYLEF | |
| CGQIILLENGKICENGTHSELMQKKGKYAQLIQKM | |
| HKEATSDMLQDTAKIAEKPKVESQALATSLEESLN | |
| GNAVPEHQLTQEEEMEEGSLSWRVYHHYIQAAGGY | |
| MVSCIIFFFVVLIVFLTIFSFWWLSYWLEQGSGTN | |
| SSRESNGTMADLGNIADNPQLSFYQLVYGLNALLL | |
| ICVGVCSSGIFTKVTRKASTALHNKLFNKVFRCPM | |
| SFFDTIPIGRLLNCFAGDLEQLDQLLPIFSEQFLV | |
| LSLMVIAVLLIVSVLSPYILLMGAIIMVICFIYYM | |
| MFKKAIGVFKRLENYSRSPLFSHILNSLQGLSSIH | |
| VYGKTEDFISQFKRLTDAQNNYLLLFLSSTRWMAL | |
| RLEIMTNLVTLAVALFVAFGISSTPYSFKVMAVNI | |
| VLQLASSFQATARIGLETEAQFTAVERILQYMKMC | |
| VSEAPLHMEGTSCPQGWPQHGEIIFQDYHMKYRDN | |
| TPTVLHGINLTIRGHEVVGIVGRTGSGKSSLGMAL | |
| FRLVEPMAGRILIDGVDICSIGLEDLRSKLSVIPQ | |
| DPVLLSGTIRFNLDPFDRHTDQQIWDALERTFLTK | |
| AISKFPKKLHTDVVENGGNFSVGERQLLCIARAVL | |
| RNSKIILIDEATASIDMETDTLIQRTIREAFQGCT | |
| VLVIAHRVTTVLNCDHILVMGNGKVVEFDRPEVLR | |
| KKPGSLFAALMATATSSLR | |
| ABCF1 | |
| Full name: | ATP-binding cassette |
| | sub-family F member 1 |
| UniProt record number: | Q8NE71 |
| Genbank mRNA sequence ref: | AF027302.1 |
| Uniprot protein sequence ref: | Q8NE71-1 |
| Uniprot protein sequence: | |
| MPKAPKQQPPEPEWIGDGESTSPSDKVVKKGKKDK | |
| KIKKTFFEELAVEDKQAGEEEKVLKEKEQQQQQQQ | |
| QQQKKKRDTRKGRRKKDVDDDGEEKELMERLKKLS | |
| VPTSDEEDEVPAPKPRGGKKTKGGNVFAALIQDQS | |
| EEEEEEEKHPPKPAKPEKNRINKAVSEEQQPALKG | |
| KKGKEEKSKGKAKPQNKFAALDNEEEDKEEEIIKE | |
| KEPPKQGKEKAKKAEQGSEEEGEGEEEEEEGGESK | |
| ADDPYAHLSKKEKKKLKKQMEYERQVASLKAANAA | |
| ENDFSVSQAEMSSRQAMLENASDIKLEKFSISAHG | |
| KELFVNADLYIVAGRRYGLVGPNGKGKTTLLKHIA | |
| NRALSIPPNIDVLLCEQEVVADETPAVQAVLRADT | |
| KRLKLLEEERRLQGQLEQGDDTAAERLEKVYEELR | |
| ATGAAAAEAKARRILAGLGFDPEMQNRPTQKFSGG | |
| WRMRVSLARALFMEPTLLMLDEPTNHLDLNAVIWL | |
| NNYLQGWRKTLLIVSHDQGFLDDVCTDIIHLDAQR | |
| LHYYRGNYMTFKKMYQQKQKELLKQYEKQEKKLKE | |
| LKAGGKSTKQAEKQTKEALTRKQQKCRRKNQDEES | |
| QEAPELLKRPKEYTVRFTFPDPPPLSPPVLGLHGV | |
| TFGYQGQKPLFKNLDFGIDMDSRICIVGPNGVGKS | |
| TLLLLLTGKLTPTHGEMRKNHRLKIGFFNQQYAEQ | |
| LRMEETPTEYLQRGFNLPYQDARKCLGRFGLESHA | |
| HTIQICKLSGGQKARVVFAELACREPDVLILDEPT | |
| NNLDIESIDALGEAINEYKGAVIVVSHDARLITET | |
| NCQLWVVEEQSVSQIDGDFEDYKREVLEALGEVMV | |
| SRPRE | |
| SLC28A3 | |
| Full name: | Solute carrier |
| | family 28 member 3 |
| UniProt record number: | Q9HAS3 |
| Genbank mRNA sequence ref: | AF305210.1 |
| Uniprot protein sequence ref: | Q9HAS3-1 |
| Uniprot protein sequence: | |
| MELRSTAAPRAEGYSNVGFQNEENFLENENTSGNN | |
| SIRSRAVQSREHTNTKQDEEQVTVEQDSPRNREHM | |
| EDDDEEMQQKGCLERRYDTVCGFCRKHKTTLRHII | |
| WGILLAGYLVMVISACVLNFHRALPLFVITVAAIF | |
| FVVWDHLMAKYEHRIDEMLSPGRRLLNSHWFWLKV | |
| VVIWSSLVLAVIFWLAFDTAKLGQQQLVSFGGLIM | |
| YIVLLFLFSKYPTRVYWRPVLWGIGLQFLLGLLIL | |
| RTDPGFIAFDWLGRQVQTFLEYTDAGASFVFGEKY | |
| KDHFFAFKVLPIVVFFSTVMSMLYYLGLMQWIIRK | |
| VGWIMLVTTGSSPIESVVASGNIFVGQTESPLLVR | |
| PYLPYITKSELHAIMTAGFSTIAGSVLGAYISFGV | |
| PSSHLLTASVMSAPASLAAAKLFWPETEKPKITLK | |
| NAMKMESGDSGNLLEAATQGASSSISLVANIAVNL | |
| IAFLALLSFMNSALSWFGNMFDYPQLSFELICSYI | |
| FMPFSFMMGVEWQDSFMVARLIGYKTFFNEFVAYE | |
| HLSKWIHLRKEGGPKFVNGVQQYISIRSEIIATYA | |
| LCGFANIGSLGIVIGGLTSMAPSRKRDIASGAVRA | |
| LIAGTVACFMTACIAGILSSTPVDINCHHVLENAF | |
| NSTFPGNTTKVIACCQSLLSSTVAKGPGEVIPGGN | |
| HSLYSLKGCCTLLNPSTFNCNGISNTF | |
| ABCB6 | |
| Full name: | ATP-binding cassette |
| | sub-family B |
| | member 6, mito. |
| UniProt record number: | Q9NP58 |
| Genbank mRNA sequence ref: | AF076775.1 |
| Uniprot protein sequence ref: | Q9NP58-1 |
| Uniprot protein sequence: | |

MVTVGNYCEAEGPVGPAWMQDGLSPCFFFTLVPST
RMALGTLALVLALPCRRRERPAGADSLSWGAGPRI
SPYVLQLLLATLQAALPLAGLAGRVGTARGAPLPS
YLLLASVLESLAGACGLWLLVVERSQARQRLAMGM
IKFRHSPGLLLLVVTVAFAAENLALVSWNSPQWWW
ARADLGQQVQFSLWVLRYVVSGGLFVLGLWAPGLR
PQSYTLQVHEEDQDVERSQVRSAAQQSTWRDFGRK
LRLLSGYLWPRGSPALQLVVLICLGLMGLERALNV
LVPIFYRNIVNLLTEKAPWNSLAWTVTSYVFLKFL
QGGGTGSTGFVSNLRTFLWIRVQQFTSRRVELLIF
SHLHELSLRWHLGRRTGEVLRIADRGTSSVTGLLS
YLVFNVIPTLADIIIGIIYFSMFFNAWFGLIVFLC
MSLYLTLTIVVTEWRTKFRRAMNTQENATRARAVD
SLLNFETVKYYNAESYEVERYREAIIKYQGLEWKS
SASLVLLNQTQNLVIGLGLLAGSLLCAYFVTEQKL
QVGDYVLFGTYIIQLYMPLNWFGTYYRMIQTNFID
MENMFDLLKEETEVKDLPGAGPLRFQKGRIEFENV
HFSYADGRETLQDVSFTVMPGQTLALVGPSGAGKS
TILRLLFRFYDISSGCIRIDGQDISQVTQASLRSH
IGVVPQDTVLFNDTIADNIRYGRVTAGNDEVEAAA
QAAGIHDAIMAFPEGYRTQVGERGLKLSGGEKQRV
AIARTILKAPGIILLDEATSALDTSNERAIQASLA
KVCANRTTIVVAHRLSTVVNADQILVIKDGCIVER
GRHEALLSRGGVYADMWQLQQGQEETSEDTKPQTM
ER

The one or more PBD-resistance genes referred to in the methods described herein may be selected from the above genes; that is selected from the group consisting of:
(a) ABCG2, ABCC2, SLCO2B1, SLC7A7, SLC22A3, SLCO2A1, ABCC12, ATP7A, AQP7, SLC5A1, SLC16A2, SLC7A9, ABCB4, ABCC11, ABCF1, SLC28A3, and ABCB6.

In order of increasing preference (ie. (j) is most preferred), the one or more PBD-resistance genes referred to in the methods described herein may be selected from the following groups consisting of:
(b) ABCG2, ABCC2, SLCO2B1, SLC7A7, SLC22A3, SLCO2A1, ABCC12, ATP7A, AQP7, and SLC5A1;
(c) ABCG2, ABCC2, SLCO2B1, SLC7A7, and SLC22A3;
(d) ABCG2, ABCC2, SLCO2B1, and SLC7A7;
(e) ABCG2, ABCC2, SLCO2B1, and SLC22A3;
(f) ABCG2, ABCC2, and SLC7A7;
(g) ABCG2, ABCC2, and SLC22A3;
(h) ABCG2, ABCC2, and SLCO2B1;
(i) ABCG2 and ABCC2; or
(j) ABCG2.

For example SLCO2B1 could be the one gene selected from group (c). Within the “one or more” definition, SLCO2B1 could then optionally be combined with any of the other (or all of the other) genes listed in group (c) (so, ABCG2, ABCC2, SLC7A7, and/or SLC22A3).

In some embodiments two or more PBD-resistance genes are selected from one of the above groups (a) to (g). For example, again using group (c), SLCO2B1 and ABCG2 could be the two genes selected from group (c), optionally in combination with any of the other (or all of the other) genes listed in group (c) (so, ABCC2, SLC7A7, and/or SLC22A3). Accordingly, in this particular example, only overexpression of both SLCO2B1 and ABCG2 would indicate that the proliferative disease is resistant to treatment with the PBD agent, or the overexpression of neither SLCO2B1 nor ABCG2 lead to selecting a subject for treatment with a PBD agent. A preferred embodiment is the selection of the two genes, ABCG2 and ABCC2.

In some embodiments three or more PBD-resistance genes are selected from one of the above groups (a) to (h).

In some embodiments four or more PBD-resistance genes are selected from one of the above groups (a) to (e).

In some embodiments five or more PBD-resistance genes are selected from one of the above groups (a) to (c).

In some embodiments ten or more PBD-resistance genes are selected from one of the above groups (a) to (b).

For the avoidance of doubt, the term “XXX or more” as used above and elsewhere herein, wherein ‘XXX’ is a whole number such as one, two, three, etc., is used to mean “XXX, or more than XXX”. That is, the term is used to describe the two alternatives:
(1) “exactly XXX” (i.e. neither more than XXX, nor less than XXX), or
(2) “more than XXX” (i.e. the ascending numerical range of whole numbers, starting from XXX+1).

### Overexpression of PBD-Resistance Genes

The term “overexpression” of a PBD-resistance gene as used herein refers to the higher level of expression of a PBD-resistance gene in a PBD-resistance proliferative cell, as compared to a control sample as defined herein.

In some embodiments, overexpression of a PBD-resistance gene is indicated by an at least 2-fold increase relative to a control sample. For example, in some embodiments overexpression of a PBD-resistance gene is indicated by an at least 5-fold, at least 10-fold, at least 20-fold, at least 50-fold, or at least 100-fold increase relative to a control sample.

In some embodiments, overexpression of a PBD-resistance gene is indicated by an increase relative to a control sample that has a p-value no greater than 0.05. For example, in some embodiments overexpression of a PBD-resistance gene is indicated by an increase relative to a control sample that has a p-value no greater than 0.01, no greater than 0.005, no greater than 0.001, no greater than 0.0005, no greater than 0.0001, no greater than 0.00005, or no greater than 0.00001 (Students T-test of the replicate 2\hat{ }(−Delta C_{T}) values for each gene in control and treated groups).

### Determining Overexpression

The expression level of a PBD-resistance gene in a sample may be determined by measuring the level of mRNA transcription from the PBD-resistance gene in a sample. For example, the level of mRNA transcription may be measured by cDNA PCR array, RT-PCR, fluorescence in situ hybridization (FISH), Southern Blot, immunohistochemisty (IHC), polymerase chain reaction (PCR), quantitative PCR (qPCR), quantitative real-time PCR (qRT-PCR), microarray based comparative genomic hybridization, comparative genomic hybridization, or ligase chain reaction (LCR).

The expression level of a PBD-resistance gene in a sample may be determined by measuring the level of protein translation from the PBD-resistance gene in a sample. That is, the expression level of a PBD-resistance gene in a sample may be determined by measuring the level of protein product produced from the PBD-resistance gene; such a protein product is described herein PBD-resistance polypeptide. The level of PBD-resistance polypeptide may be measured by contacting the sample with an antibody that specifically binds the PBD-resistance polypeptide (described herein as an anti-PBD-resistance antibody) and binding of the anti-PBD-resistance antibody to PBD-resistance polypeptide. An example of such a method is Western blotting.

The expression level of a PBD-resistance gene in a sample may be determined by measuring the activity of the PBD-resistance polypeptide produced from the PBD-resistance gene. Preferably the activity will be readily and directly measureable by a straightforward assay. For example, most of the PBD-resistance genes described herein have either a transporter or enzymatic activity; these activities are measurable through a range of assays available to the skilled person (see, for example, Xie H., Acta Biochim Biophys Sin., 2008 April; 40(4):269-77; Volpe D., Expert Opinion on Drug Discovery, Vol. 11(1), 2016, pp. 91-103).

### Control Samples

In some embodiments, the expression level of a PBD-resistance gene in a test sample from a subject is compared to the expression level in a control sample. Controls are useful to identify experimental artefacts.

In some cases, the control may be a reference sample or reference dataset. The reference may be a sample that has been previously obtained from a subject with a known degree of suitability (i.e. sensitivity to PDB agents). The reference may be a dataset obtained from analyzing a reference sample.

In some, embodiments the control or reference sample is from non-PBD resistant proliferative cells. In some embodiments the control or reference sample is from non-PBD resistant non-proliferative cells.

Controls may be positive controls in which the PBD-resistance gene is known to be overexpressed and/or are derived from PBD-resistant proliferative cells. Controls may be negative controls in which the PBD-resistance gene is known to be absent or expressed at low level.

Controls may be samples of tissue that are from subjects who are known to benefit from the treatment; for example, the control samples may be from subjects whose disorders are known not to be PBD-resistant, or whose disorders are known to have normal expression of the PBD-resistance gene.

The tissue of the control sample may be of the same type as the test sample. For example, a test sample of tumor tissue from a subject may be compared to a control sample of tumor tissue from a subject whose disorder is known not to be PBD-resistant, such as a individual who has previously responded to treatment to treatment with a PBD agent.

In some cases the control may be a sample obtained from the same subject as the test sample, but from a tissue known to be healthy. Thus, a sample of cancerous tissue from a subject may be compared to a non-cancerous tissue sample.

In some cases, the control is internal to the test sample. For example, the control may be the level of expression of a gene known to be constitutively expressed at a constant rate. Typical examples of such ‘housekeeping’ are genes that are required for the maintenance of basal cellular functions that are essential for the existence of a cell, regardless of its specific role in the tissue or organism (see Silver et al., BMC Mol Biol. 2006; 7: 33).

In some cases, the control is a cell culture sample.

In some protein expression cases, a test sample is analyzed prior to incubation with an antibody to determine the level of background staining inherent to that sample.

In some protein expression cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern.

### Antagonists of PBD-Resistance Genes

An antagonist of a PBD-resistant gene as described herein is an agent which, when administered to a subject, decreases the expression of one of more PBD resistance gene. Preferably the antagonist prevents or reduces overexpression of one or more PBD-resistant gene such that the PBD resistance of a PBD-resistant proliferative disorder is reduced or abrogated.

The antagonist may reduce PBD gene expression by reducing the level of mRNA transcription from one of more PBD resistance gene. The antagonist may reduce PBD gene expression by reducing the expression of one or more PBD-resistance polypeptide. The antagonist may reduce PBD gene expression by reducing the activity of one or more PBD-resistance polypeptide.

Antagonists that are useful in the methods include:
a) RNA based antagonists (eg. RNA interference, RNAi) that target the transcription and/or translation of one or more PBD resistance genes, such as antisense RNA, miRNA, siRNA, and shRNAs;
b) Mimetics or analogs of the antagonists of (a)
c) Agents that increase the expression of the antagonists of (a)
d) PBD-resistance polypeptide binding agents such as anti-PBD resistance antibodies; and
e) organic/small molecule inhibitors of one or more PBD-resistance polypeptides, such as molecules which inhibit the transporter or enzymatic activity of one or more PBD-resistance polypeptides.

For example, the expression of several of the PBD-resistance genes disclosed herein has been shown to be regulated by specific miRNAs (see Haenisch et al., Br J Clin Pharmacol., 77:4, 587-596, summarized below):

| PBD-resistance gene | Inhibitory miRNAs |
|---|---|
| ABCG2 | miR-200c, miR-212, miR-328, |
| | miR-519c, miR-520h |
| ABCC2 | miR-297, miR-379 |

“Organic/small molecule inhibitors of one or more PBD-resistance polypeptides” means any chemical compound or biological molecule that reduces the PBD-resistance conferring activity of a PBD-resistance polypeptide. For example, a small molecule inhibitor that reduced the transporter activity of one or more of the ABC transporter proteins described herein.

To examine the extent of inhibition of, e.g., ABC activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%.

Specific organic/small molecule inhibitors of one or more PBD-resistance polypeptides suitable for use in the present disclosure include:
a) MK-571
   i. CAS Number→115104-28-4 [115103-85-0, sodium salt]
      (see http://www.cas.org/content/chemical-substances/faqs)
   ii. Unique Ingredient Identifier (UNII)→Q8W8588793
      (see http://www.fda.gov/ForIndustry/DataStandards/SubstanceRegisration System-UniqueIngredientIdentifierUNII/default.htm)

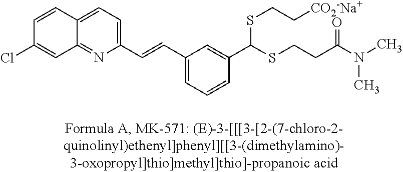

b) Biricodar
   i. CAS Number→174254-13-8
      (see http://www.cas.org/content/chemical-substances/faqs)
   ii. Unique Ingredient Identifier (UNII)→9WQP0L619L
      (see http://www.fda.gov/ForIndustry/DataStandards/SubstanceRegistration System-UniqueIngredientIdentifierUNII/default.htm)

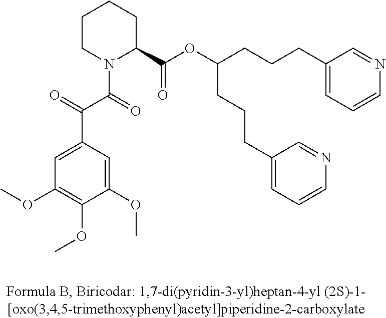 [SMILES: COc1cc(C(=O)C(=O)N2CCCC[C@H]2C(=O)OC(CCCc2cccnc2)CCCc2cccnc2)cc(OC)c1OC]

c) Probenecid
   i. CAS Number→57-66-9
      (see http://www.cas.org/content/chemical-substances/faqs)
   ii. Unique Ingredient Identifier (UNII)→PO572Z7917
      (see http://www.fda.gov/ForIndustry/DataStandards/SubstanceRegistration System-UniqueIngredientIdentifierUNII/default.htm)

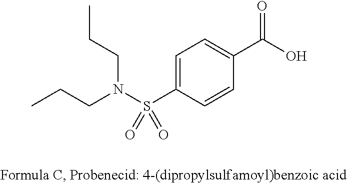 [SMILES: CCCN(CCC)S(=O)(=O)c1ccc(C(=O)O)cc1]

d) Reversan
   i. CAS Number→313397-13-6
      (see http://www.cas.org/content/chemical-substances/faqs)

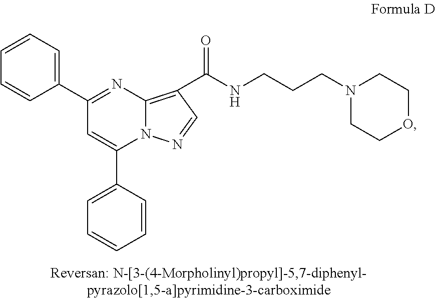 [SMILES: O=C(NCCCN1CCOCC1)c1cnn2c(-c3ccccc3)cc(-c3ccccc3)nc12]

e) Fumitremorgin C
   i. CAS Number→118974-02-0
      (see http://www.cas.org/content/chemical-substances/faqs)

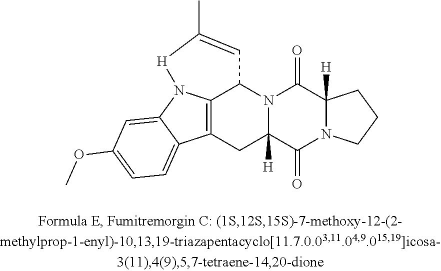

f) Ko143
   i. CAS Number→461054-93-3
      (see http://www.cas.org/content/chemical-substances/faqs)

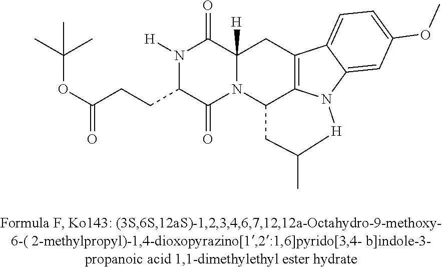

g) Ritonavir
   i. CAS Number→155213-67-5

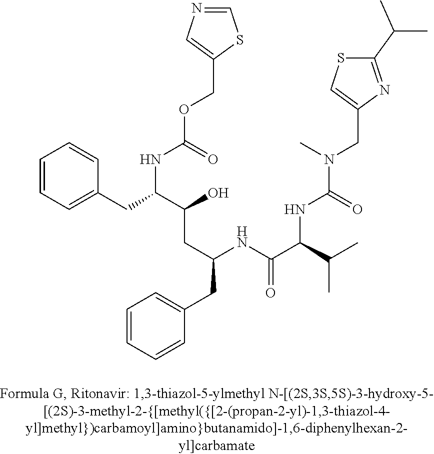 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

h) Saquinavir
   i. CAS Number→127779-20-8

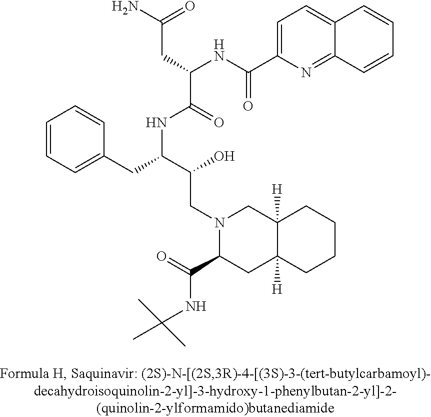 [SMILES: CC(C)(C)NC(=O)[C@@H]1C[C@@H]2CCCC[C@@H]2CN1C[C@@H](O)[C@H](Cc1ccccc1)NC(=O)[C@H](CC(N)=O)NC(=O)c1ccc2ccccc2n1]

i) Lamivudine
   i. CAS Number→127779-20-8

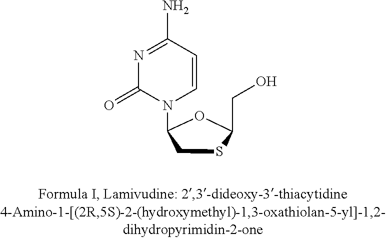

j) Abacavir
   i. CAS Number→136470-78-5

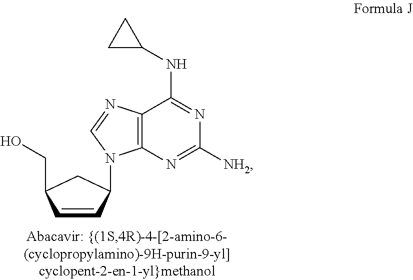 [SMILES: Nc1nc(NC2CC2)c2ncn([C@H]3C=C[C@@H](CO)C3)c2n1]

k) Emtricitabine
   i. CAS Number→143491-57-0

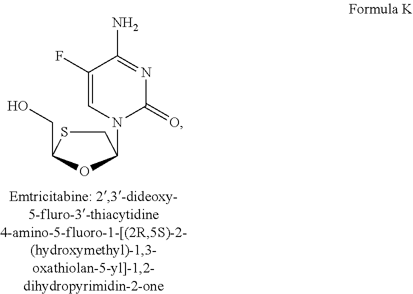 [SMILES: Nc1nc(=O)n([C@@H]2CS[C@H](CO)O2)cc1F]

l) Efavirenz
   i. CAS Number→154598-52-4

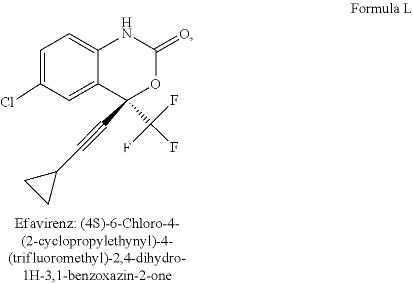 [SMILES: O=C1Nc2ccc(Cl)cc2[C@@](C#CC2CC2)(C(F)(F)F)O1]

m) Delavirdine
   i. CAS Number→136817-59-9

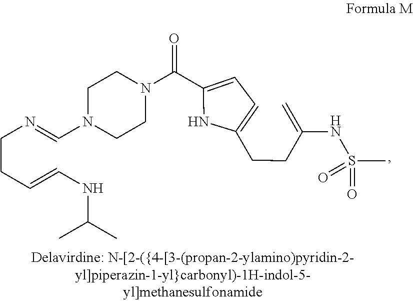 [SMILES: C=C(CCc1ccc(C(=O)N2CCN(/C=N/CC/C=C/NC(C)C)CC2)[nH]1)NS(C)(=O)=O]

n) Nevirapine
   i. CAS Number→129618-40-2

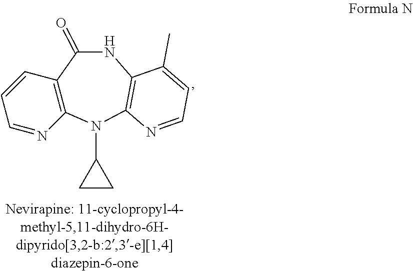 [SMILES: Cc1ccnc2c1NC(=O)c1cccnc1N2C1CC1]

o) Cidofovir
   i. CAS Number→113852-37-2

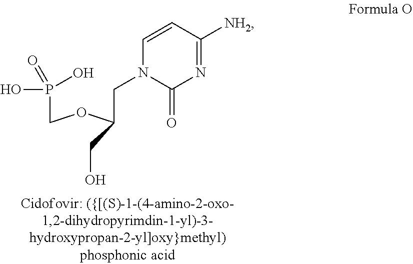 [SMILES: Nc1ccn(C[C@@H](CO)OCP(=O)(O)O)c(=O)n1]

p) Adefovir
   i. CAS Number→106941-25-7

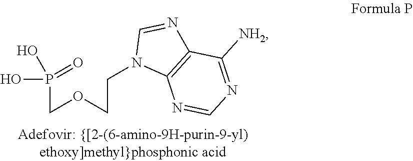 [SMILES: Nc1ncnc2c1ncn2CCOCP(=O)(O)O]

q) Tenofovir
   i. CAS Number→201341-05-1

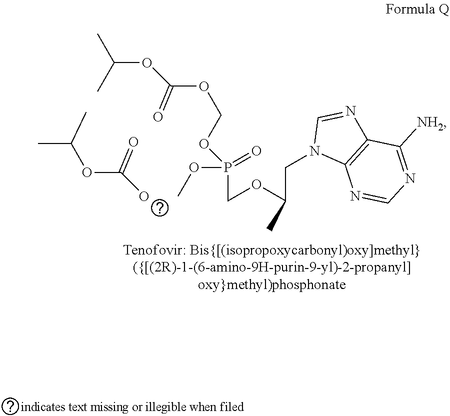

r) Cyclosporine
   i. CAS Number→59865-13-3

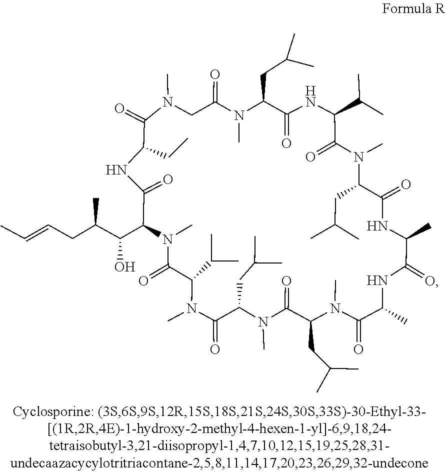 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

s) PSC833
   i. CAS Number→121584-18-7

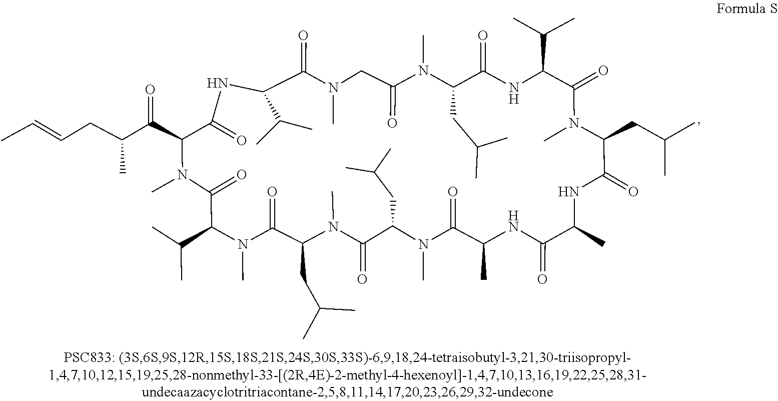 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@@H](C)C(=O)N(C)[C@@H](CC(C)C)C(=O)N(C)[C@@H](CC(C)C)C(=O)N(C)[C@@H](C(C)C)C(=O)N1C]

t) Febuxostat
   i. CAS Number→144060-53-7

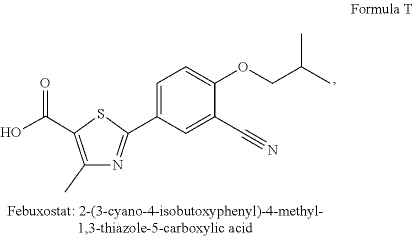 [SMILES: Cc1nc(-c2ccc(OCC(C)C)c(C#N)c2)sc1C(=O)O]

u) Elacridar
   i. CAS Number→143664-11-3

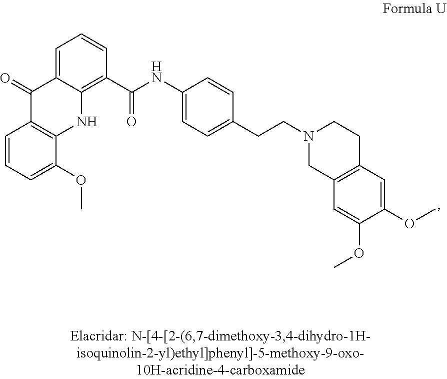 [SMILES: COc1cc2c(cc1OC)CN(CCc1ccc(NC(=O)c3cccc4c(=O)c5cccc(OC)c5[nH]c34)cc1)CC2]

v) Tariquidar
   i. CAS Number→206873-63-4

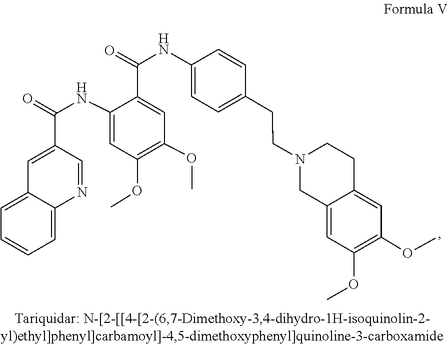 [SMILES: COc1cc2c(cc1OC)CN(CCc1ccc(NC(=O)c3cc(OC)c(OC)cc3NC(=O)c3cnc4ccccc4c3)cc1)CC2]

### Pyrrolobenzodiazepine Agent

The term pyrrolobenzodiazepine (PBD) agent is used herein to describe a chemotherapeutic comprising a PBD moiety. PBD moieties have the general structure:

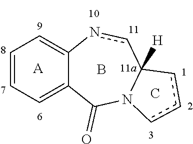

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring.

### PBD Compounds

In some embodiments the PBD-agent is, or comprises, a PBD-compound selected from the group consisting of:

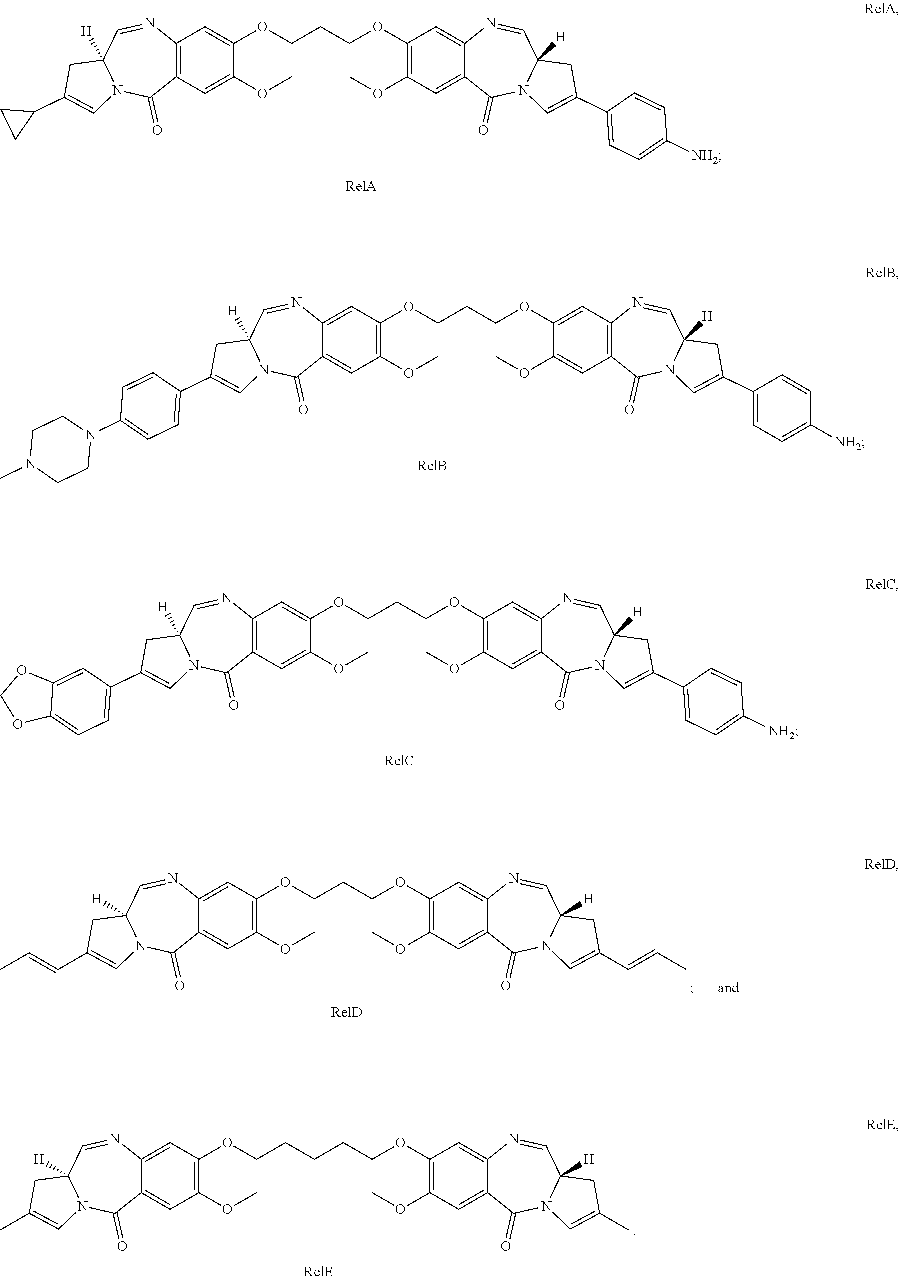

The PBD-compound RelE is particularly preferred.

### PBD Cell Binding Agents (PBD-CBA)

As used herein, the term “PBD-CBA” refers to a PBD agent comprising a PBD compound conjugated to a cell binding agent, such as an antibody (Ab). In a first aspect, the PBD-CBA has the structure defined in the following paragraphs:

1. A conjugate of formula L-(D^{L})ₚ, where D^{L} is of formula I or II:

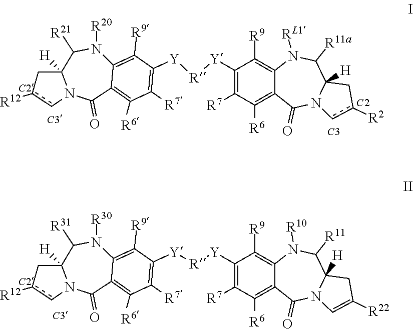

wherein:
L is a cell binding agent (CBA);
when there is a double bond present between C2′ and C3′, R¹² is selected from the group consisting of:
   (ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
   (ib) C₁₋₅ saturated aliphatic alkyl;
   (ic) C₃₋₆ saturated cycloalkyl;

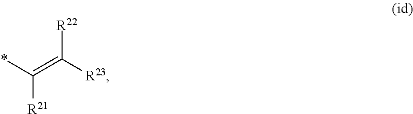

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

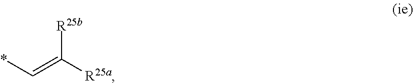

wherein one of R^{25a} and R^{25b} is H and the other is selected from: phenyl, which phenyl is optionally substituted by a group selected from halo, methyl, methoxy; pyridyl; and thiophenyl; and

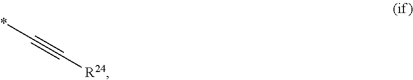

where R²⁴ is selected from: H; C₁₋₃ saturated alkyl; C2-3 alkenyl; C2-3 alkynyl;

cyclopropyl; phenyl, which phenyl is optionally substituted by a group selected from halo, methyl, methoxy; pyridyl; and thiophenyl;

when there is a single bond present between C2′ and C3′,

### R¹² is

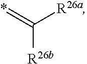

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the cell binding agent (CBA);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

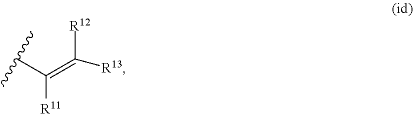

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

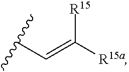

wherein one of R^{15a} and R^{15b} is H and the other is selected from: phenyl, which phenyl is optionally substituted by a group selected from halo, methyl, methoxy; pyridyl; and thiophenyl; and

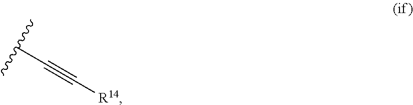

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

### R² is

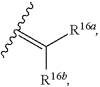

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

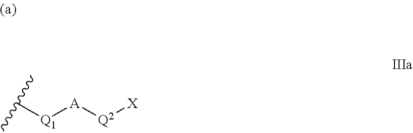

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

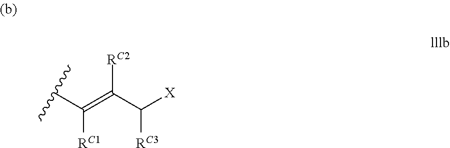

where;
R^{C1}, R^{C2} and R^{C3} are independently selected from H and unsubstituted C₁₋₂ alkyl;

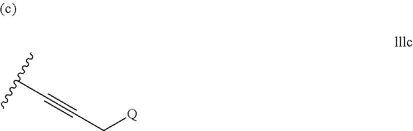

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, NH—C(═O)—R^{L2′}, NHNH—R^{L2′}, CONHNH—R^{L2′},

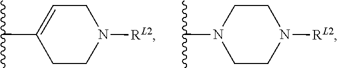

NR^{N}R^{L2′}, wherein R^{N} is selected from the group comprising H and C₁₋₄ alkyl;
R^{L2′} is a linker for connection to the cell binding agent (CBA);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

2. The conjugate according to statement 1, wherein the conjugate is not:

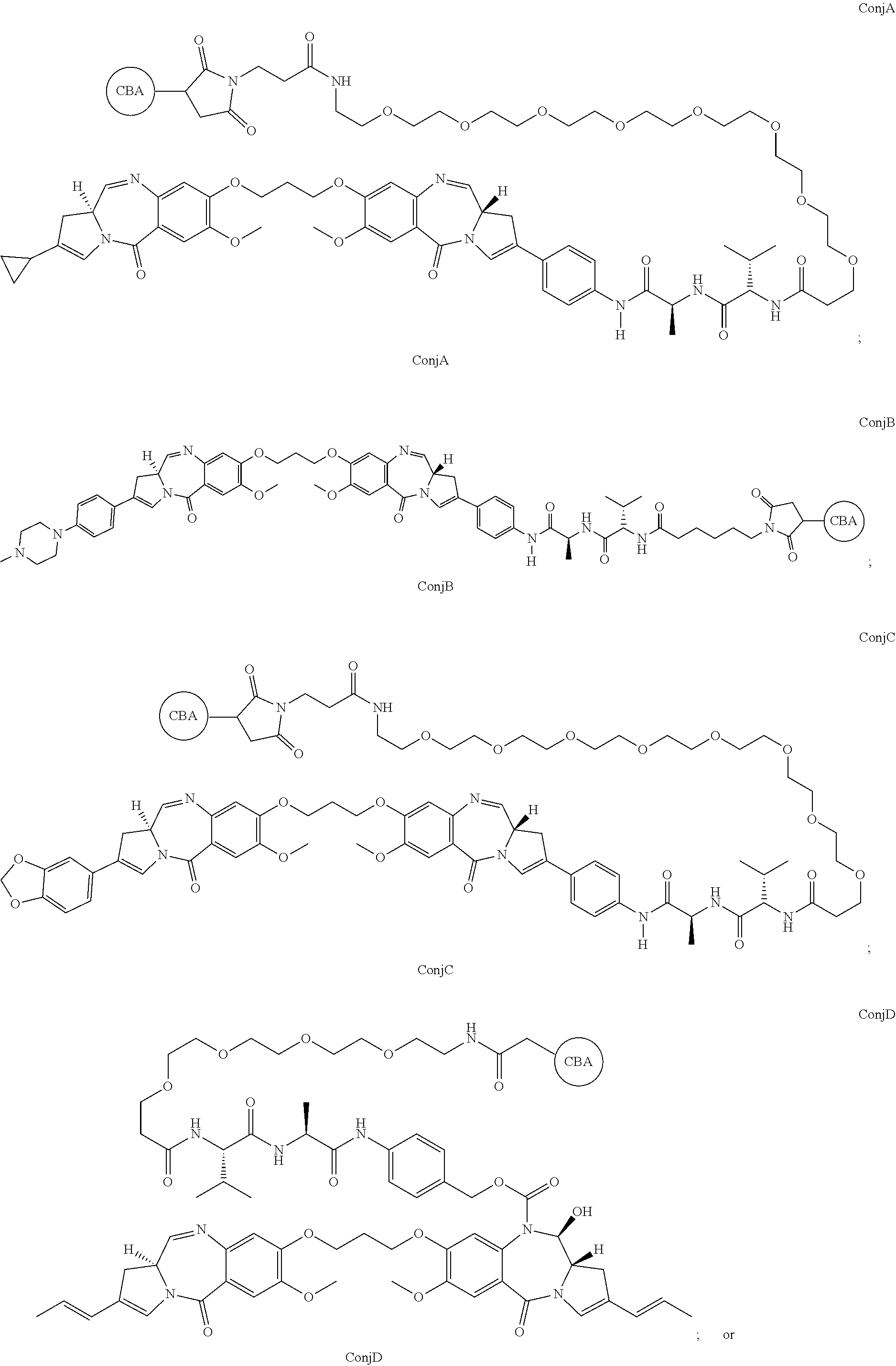

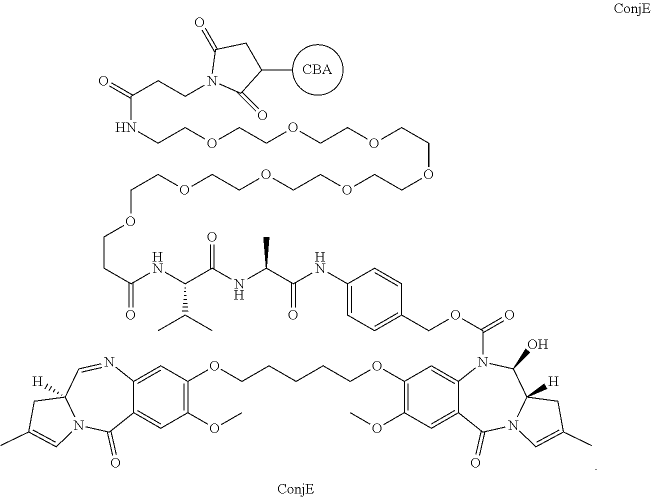

3. The conjugate according to either statement 1 or statement 2, wherein R⁷ is selected from H, OH and OR.

4. The conjugate according to statement 3, wherein R⁷ is a C₁₋₄ alkyloxy group.

5. The conjugate according to any one of statements 1 to 4, wherein Y is O.

6. The conjugate according to any one of the preceding statements, wherein R″ is C₃₋₇ alkylene.

7. The conjugate according to any one of statements 1 to 6, wherein R⁹ is H.

8. The conjugate according to any one of statements 1 to 7, wherein R⁶ is selected from H and halo.

9. The conjugate according to any one of statements 1 to 8, wherein there is a double bond between C2′ and C3′, and R¹² is a C₅₋₇ aryl group.

10. The conjugate according to statement 9, wherein R¹² is phenyl.

11. The conjugate according to any one of statements 1 to 8, wherein there is a double bond between C2′ and C3′, and R¹² is a C₈₋₁₀ aryl group.

12. The conjugate according to any one of statements 9 to 11, wherein R¹² bears one to three substituent groups.

13. The conjugate according to any one of statements 9 to 12, wherein the substituents are selected from methoxy, ethoxy, fluoro, chloro, cyano, bis-oxy-methylene, methyl-piperazinyl, morpholino and methyl-thiophenyl.

14. The conjugate according to any one of statements 1 to 8, wherein there is a double bond between C2′ and C3′, and R¹² is a C₁₋₅ saturated aliphatic alkyl group.

15. A compound according to statement 16, wherein R¹² is methyl, ethyl or propyl.

16. The conjugate according to any one of statements 1 to 8, wherein there is a double bond between C2′ and C3′, and R¹² is a C₃₋₆ saturated cycloalkyl group.

17. The conjugate according to statement 16, wherein R¹² is cyclopropyl.

18. The conjugate according to any one of statements 1 to 8, wherein there is a double bond between C2′ and C3′, and R¹² is a group of formula:

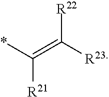

19. The conjugate according to statement 18, wherein the total number of carbon atoms in the R¹² group is no more than 4.

20. The conjugate according to statement 19, wherein the total number of carbon atoms in the R¹² group is no more than 3.

21. The conjugate according to any one of statements 18 to 20, wherein one of R²¹, R²² and R²³ is H, with the other two groups being selected from H, C₁₋₃ saturated alkyl, C₂₋₃ alkenyl, C₂₋₃ alkynyl and cyclopropyl.

22. The conjugate according to any one of statements 18 to 20, wherein two of R²¹, R²² and R²³ are H, with the other group being selected from H, C₁₋₃ saturated alkyl, C₂₋₃ alkenyl, C₂₋₃ alkynyl and cyclopropyl.

23. The conjugate according to any one of statements 1 to 8, wherein there is a double bond between C2′ and C3′, and R¹² is a group of formula:

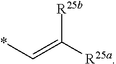

24. The conjugate according to statement 23, wherein R¹² is the group:

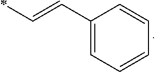 [SMILES: */C=C/c1ccccc1]

25. The conjugate according to any one of statements 1 to 8, wherein there is a double bond between C2′ and C3′, and R¹² is a group of formula:

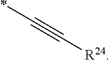

26. The conjugate according to statement 25, wherein R²⁴ is selected from H, methyl, ethyl, ethenyl and ethynyl.

27. The conjugate according to statement 26, wherein R²⁴ is selected from H and methyl.

28. The conjugate according to any one of statements 1 to 8, wherein there is a single bond between C2′ and C3′, R¹² is

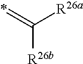

and R^{26a} and R^{26b} are both H.

29. The conjugate according to any one of statements 1 to 8, wherein there is a single bond between C2′ and C3′, R¹² is

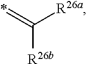

and R^{26a} and R^{26b} are both methyl.

30. The conjugate according to any one of statements 1 to 8, wherein there is a single bond between C2′ and C3′, R¹² is

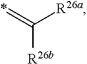

one of R^{26a} and R^{26b} is H, and the other is selected from C₁₋₄ saturated alkyl, C₂₋₃ alkenyl, which alkyl and alkenyl groups are optionally substituted.

### [Formula I]

31. The conjugate according to any one of statements 1 to 30, wherein there is a double bond between C2 and C3, and R² is a C₅₋₇ aryl group.

32. The conjugate according to statement 31, wherein R² is phenyl.

33. The conjugate according to any one of statements 1 to 30, wherein there is a double bond between C2 and C3, and R¹ is a C₈₋₁₀ aryl group.

34. A compound according to any one of statements 31 to 33, wherein R² bears one to three substituent groups.

35. The conjugate according to any one of statements 31 to 34, wherein the substituents are selected from methoxy, ethoxy, fluoro, chloro, cyano, bis-oxy-methylene, methyl-piperazinyl, morpholino and methyl-thiophenyl.

36. The conjugate according to any one of statements 1 to 30, wherein there is a double bond between C2 and C3, and R² is a C₁₋₅ saturated aliphatic alkyl group.

37. The conjugate according to statement 36, wherein R² is methyl, ethyl or propyl.

38. The conjugate according to any one of statements 1 to 30, wherein there is a double bond between C2 and C3, and R² is a C₃₋₆ saturated cycloalkyl group.

39. The conjugate according to statement 38, wherein R² is cyclopropyl.

40. The conjugate according to any one of statements 1 to 30, wherein there is a double bond between C2 and C3, and R² is a group of formula:

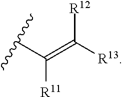

41. The conjugate according to statement 40, wherein the total number of carbon atoms in the R² group is no more than 4.

42. The conjugate according to statement 41, wherein the total number of carbon atoms in the R² group is no more than 3.

43. The conjugate according to any one of statements 40 to 42, wherein one of R¹¹, R¹² and R¹³ is H, with the other two groups being selected from H, C₁₋₃ saturated alkyl, C₂₋₃ alkenyl, C₂₋₃ alkynyl and cyclopropyl.

44. The conjugate according to any one of statements 40 to 42, wherein two of R¹¹, R¹² and R¹³ are H, with the other group being selected from H, C₁₋₃ saturated alkyl, C₂₋₃ alkenyl, C₂₋₃ alkynyl and cyclopropyl.

45. The conjugate according to any one of statements 1 to 30, wherein there is a double bond between C2 and C3, and R² is a group of formula:

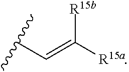

46. The conjugate according to statement 45, wherein R² is the group:

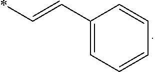 [SMILES: */C=C/c1ccccc1]

47. The conjugate according to any one of statements 1 to 30, wherein there is a double bond between C2 and C3, and R² is a group of formula:

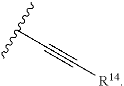

48. The conjugate according to statement 48, wherein R¹⁴ is selected from H, methyl, ethyl, ethenyl and ethynyl.

49. The conjugate according to statement 48, wherein R¹⁴ is selected from H and methyl.

50. The conjugate according to any one of statements 1 to 30, wherein there is a single bond between C2 and C3, R² is

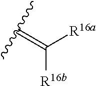

and R^{16a} and R^{16b} are both H.

51. The conjugate according to any one of statements 1 to 30, wherein there is a single bond between C2 and C3, R² is

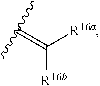

and R^{16a} and R^{16b} are both methyl.

52. The conjugate according to any one of statements 1 to 30, wherein there is a single bond between C2 and C3, R² is

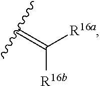

one of R^{16a} and R^{16b} is H, and the other is selected from C₁₋₄ saturated alkyl, C₂₋₃ alkenyl, which alkyl and alkenyl groups are optionally substituted.

53. The conjugate according to any one of statements 1 to 52, wherein R^{11a} is OH.

54. The conjugate according to any one of statements 1 to 53, wherein R²¹ is OH.

55. The conjugate according to any one of statements 1 to 53, wherein R²¹ is OMe.

56. The conjugate according to any one of statements 1 to 55, wherein R²⁰ is H.

57. The conjugate according to any one of statements 1 to 55, wherein R²⁰ is R^{C}.

58. The conjugate according to statement 57, wherein R^{C} is selected from the group consisting of: Alloc, Fmoc, Boc, Troc, Teoc, Psec, Cbz and PNZ.

60. The conjugate according to statement 57, wherein R^{C} is a group:

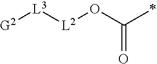

where the asterisk indicates the point of attachment to the N10 position, G² is a terminating group, L³ is a covalent bond or a cleavable linker L¹, L² is a covalent bond or together with OC(═O) forms a self-immolative linker.

61. The conjugate according to statement 60, wherein G² is Ac or Moc or is selected from the group consisting of: Alloc, Fmoc, Boc, Troc, Teoc, Psec, Cbz and PNZ.

62. The conjugate according to any one of statements 1 to 53, wherein R²⁰ and R²¹ together form a double bond between the nitrogen and carbon atoms to which they are bound.

### [Formula II]

63. The conjugate according to any one of statements 1 to 30, wherein R²² is of formula IIIa, and A is phenyl.

64. The conjugate according to any one of statements 1 to 30 and statement 63, wherein R²² is of formula IIa, and Q¹ is a single bond.

65. The conjugate according to statement 63, wherein Q² is a single bond.

66. The conjugate according to statement 63, wherein Q² is —Z—(CH₂)ₙ—, Z is O or S and n is 1 or 2.

67. The conjugate according any one of statements 1 to 30 and statement 63, wherein R²² is of formula IIIa, and Q¹ is —CH═CH—.

68. The conjugate according to any one of statements 1 to 30, wherein R²² is of formula IIIb,

and R^{C1}, R^{C2} and R^{C3} are independently selected from H and methyl.

69. The conjugate according to statement 68, wherein R^{C1}, R^{C2} and R^{C3} are all H.

70. The conjugate according to statement 68, wherein R^{C1}, R^{C2} and R^{C3} are all methyl.

71. The conjugate according to any one of statements 1 to 30 and statements 63 to 70, wherein R²² is of formula IIIa or formula IIIb and X is selected from O—R^{L2′}, S—R^{L2′}, CO₂—R^{L2′}, —N—C(═O)—R^{L2′} and NH—R^{L2′}.

72. The conjugate according to statement 71, wherein X is NH—R^{L2′}.

73. The conjugate according to any one of statements 1 to 30, wherein R²² is of formula IIIc, and Q is NR^{N}—R^{L2′}.

74. The conjugate according to statement 73, wherein R^{N} is H or methyl.

75. The conjugate according to any one of statements 1 to 30, wherein R²² is of formula IIIc, and Q is O—R^{L2′} or S—R^{L2′}.

76. The conjugate according to any one of statements 1 to 30 and statements 63 to 75, wherein R¹¹ is OH.

77. The conjugate according to any one of statements 1 to 30 and statements 63 to 75, wherein R¹¹ is OMe.

78. The conjugate according to any one of statements 1 to 30 and statements 63 to 77, wherein R¹⁰ is H.

79. The conjugate according to any one of statements 1 to 30 and statements 63 to 75, wherein R¹⁰ and R¹¹ together form a double bond between the nitrogen and carbon atoms to which they are bound.

80. The conjugate according to any one of statements 1 to 30 and statements 63 to 79, wherein R³¹ is OH.

81. The conjugate according to any one of statements 1 to 30 and statements 63 to 79, wherein R³¹ is OMe.

82. The conjugate according to any one of statements 1 to 30 and statements 63 to 81, wherein R³⁰ is H.

83. The conjugate according to any one of statements 1 to 30 and statements 63 to 79, wherein R³⁰ and R³¹ together form a double bond between the nitrogen and carbon atoms to which they are bound.

84. The conjugate according to any one of statements 1 to 83, wherein R^{6′}, R^{7′}, R^{9′}, and Y′ are the same as R⁶, R⁷, R⁹, and Y.

85. The conjugate according to any one of statements 1 to 84 wherein, wherein L-R^{L1′} or L-R^{L2′} is a group:

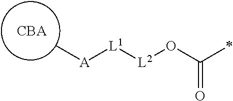

where the asterisk indicates the point of attachment to the PBD, CBA is the cell binding agent, L¹ is a cleavable linker, A is a connecting group connecting L¹ to the antibody, L² is a covalent bond or together with —OC(═O)— forms a self-immolative linker.

86. The conjugate of statement 85, wherein L¹ is enzyme cleavable.

87. The conjugate of statement 85 or statement 86, wherein L¹ comprises a contiguous sequence of amino acids.

88. The conjugate of statement 87, wherein L¹ comprises a dipeptide and the group —X₁-X₂— in dipeptide, —NH—X₁-X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-.

89. The conjugate according to statement 88, wherein the group —X₁—X₂— in dipeptide, —NH—X₁-X₂—CO—, is selected from:
-Phe-Lys-,
-Val-Ala-,
-Val-Lys-,
-Ala-Lys-,
-Val-Cit-.

90. The conjugate according to statement 89, wherein the group —X₁—X₂— in dipeptide, —NH—X₁-X₂—CO—, is -Phe-Lys-, -Val-Ala- or -Val-Cit-.

91. The conjugate according to any one of statements 88 to 90, wherein the group X₂—CO— is connected to L².

92. The conjugate according to any one of statements 88 to 91, wherein the group NH—X₁— is connected to A.

93. The conjugate according to any one of statements 88 to 92, wherein L² together with OC(═O) forms a self-immolative linker.

94. The conjugate according to statement 93, wherein C(═O)O and L² together form the group:

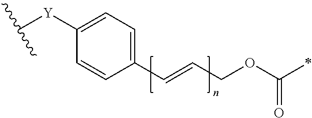

where the asterisk indicates the point of attachment to the PBD, the wavy line indicates the point of attachment to the linker L¹, Y is NH, O, C(═O)NH or C(═O)O, and n is 0 to 3.

95. The conjugate according to statement 94, wherein Y is NH.

96. The conjugate according to statement 94 or statement 95, wherein n is 0.

97. The conjugate according to statement 95, wherein L¹ and L² together with —OC(═O)— comprise a group selected from:

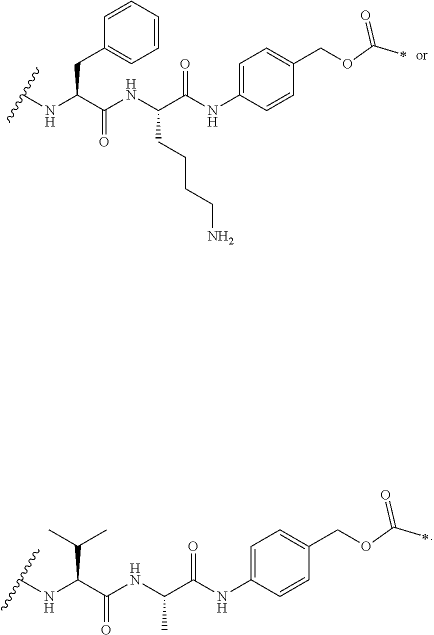

where the asterisk indicates the point of attachment to the PBD, and the wavy line indicates the point of attachment to the remaining portion of the linker L¹ or the point of attachment to A.

98. The conjugate according to statement 97, wherein the wavy line indicates the point of attachment to A.

99. The conjugate according to any one of statements 85 to 98, wherein A is:

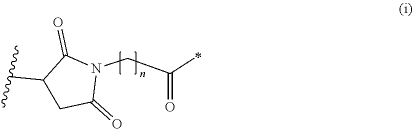

where the asterisk indicates the point of attachment to L¹, the wavy line indicates the point of attachment to the antibody, and n is 0 to 6; or

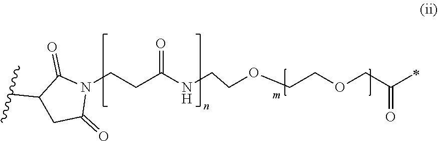

where the asterisk indicates the point of attachment to L¹, the wavy line indicates the point of attachment to the antibody, n is 0 or 1, and m is 0 to 30.

100. A conjugate according to statement 1 of formula

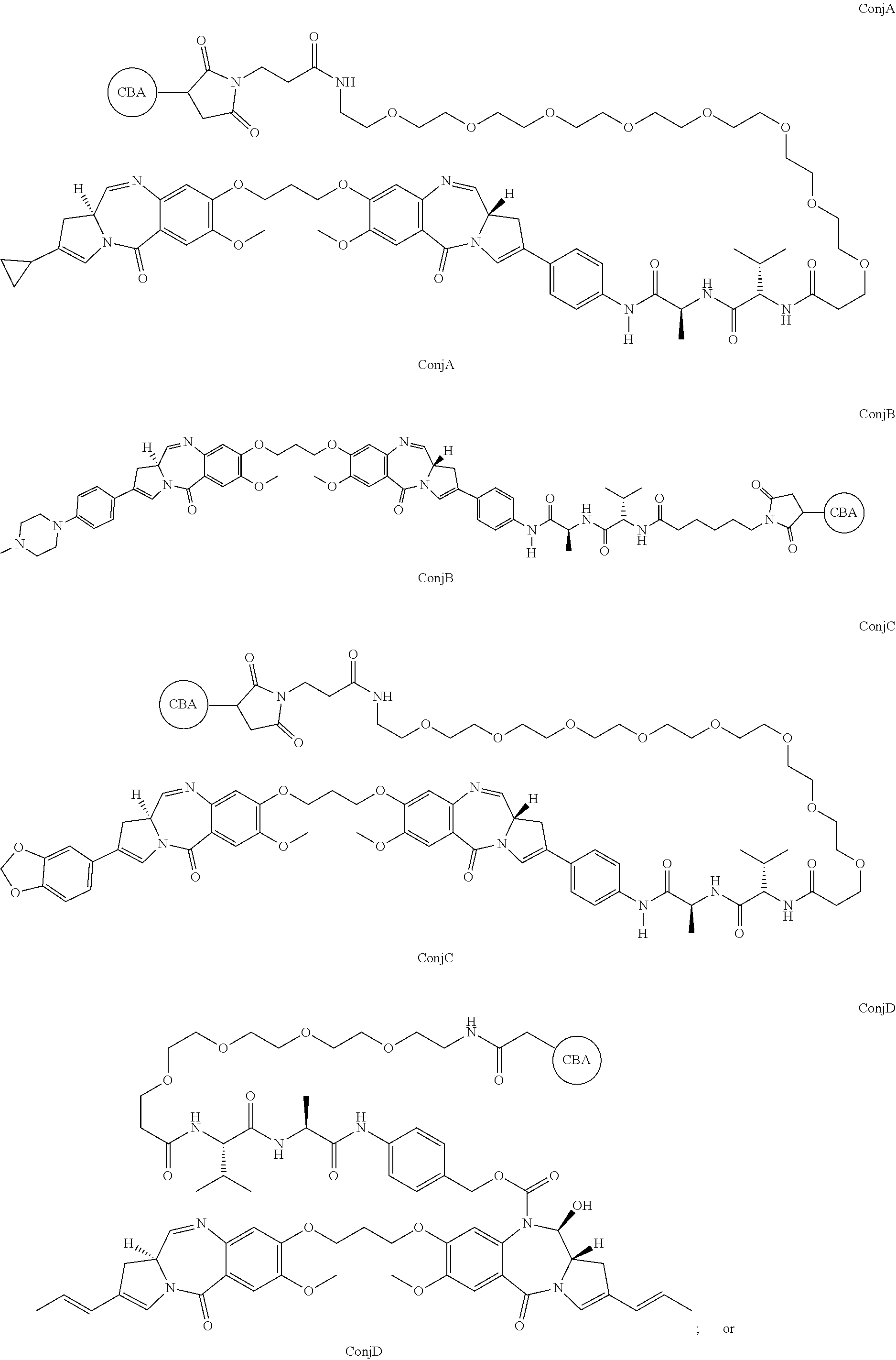

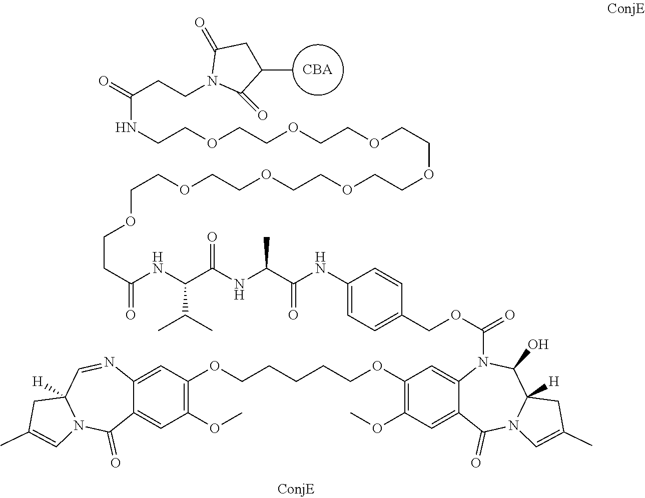

101. The conjugate according to any one of statements 1 to 100 wherein the cell binding agent (CBA) is an antibody (Ab).

The definition of the terms used in the above statements are as defined in WO2014/057119.

Conjugates having the structure of ConjE are particularly preferred.

In a second aspect, the PBD-CBA has the structure defined in the following paragraphs:

1. A conjugate of formula (III):

L-(DL)ₚ (III)

wherein:
L is a cell binding agent (CBA);

### DL is

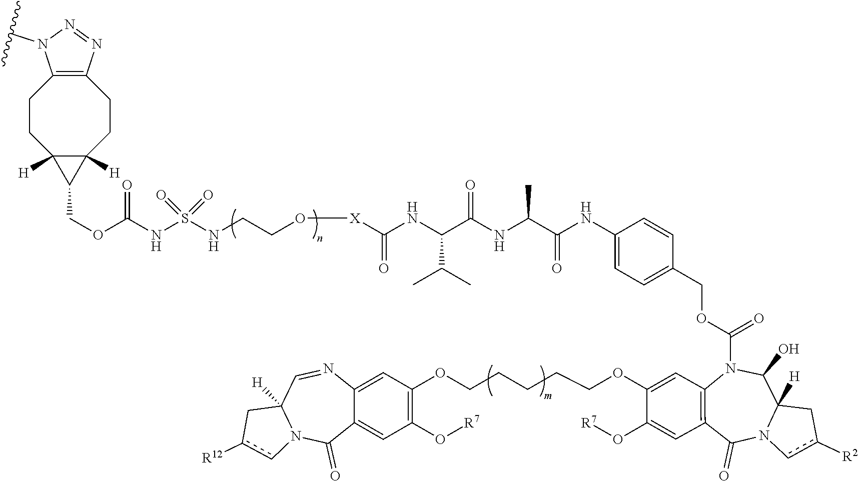

wherein:
X is selected from the group comprising: a single bond, —CH₂— and —C₂H₄—;
n is from 1 to 8;
m is 0 or 1;
R⁷ is either methyl or phenyl;

when there is a double bond between C2 and C3, R² is selected the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

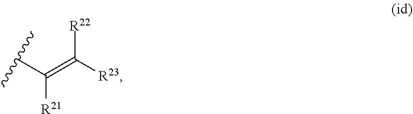

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R² group is no more than 5;

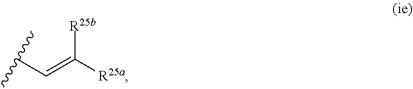

wherein one of R^{25a} and R^{25b} is H and the other is selected from: phenyl, which phenyl is optionally substituted by a group selected from halo, methyl, methoxy; pyridyl; and thiophenyl; and

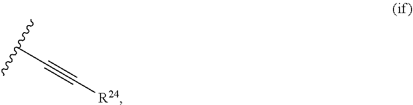

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2 and C3, R² is

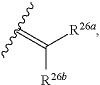

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
when there is a double bond between C2′ and C3′, R¹² is selected the group consisting of:
(iia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

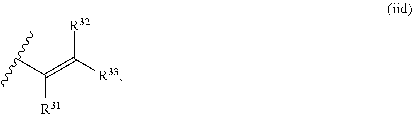

wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R¹² group is no more than 5;

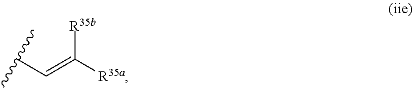

wherein one of R^{35a} and R^{35b} is H and the other is selected from: phenyl, which phenyl is optionally substituted by a group selected from halo, methyl, methoxy; pyridyl; and thiophenyl; and

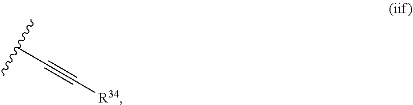

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2′ and C3′, R¹² is

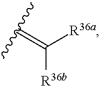

where R^{36a} and R^{36b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{36a} and R^{36b} is H, the other is selected from nitrile and a C₁₋₄ alkyl ester;
and p is from 1 to 8.

2. The conjugate according to statement 1, wherein X is a single bond.

3. The conjugate according to statement 1, wherein X is —CH₂—.

4. The conjugate according to statement 1, wherein X is —C₂H₄—.

5. The conjugate according to any one of statements 1 to 4, wherein n is 1 to 4.

6. The conjugate according to statement 5, wherein n is 1.

7. The conjugate according to statement 5, wherein n is 2.

8. The conjugate according to statement 5, wherein n is 4.

9. A compound according to any one of statements 1 to 8, wherein there is a double bond between C2 and C3, and R² is a C₅₋₇ aryl group.

10. A compound according to statement 9, wherein R² is phenyl.

11. A compound according to any one of statements 1 to 8, wherein there is a double bond between C2 and C3, and R² is a C₈₋₁₀ aryl group.

12. A compound according to any one of statements 9 to 11, wherein R² bears one to three substituent groups.

13. A compound according to any one of statements 9 to 12, wherein the substituents are selected from methoxy, ethoxy, fluoro, chloro, cyano, bis-oxy-methylene, methyl-piperazinyl, morpholino and methyl-thiophenyl.

14. A compound according to any one of statements 1 to 8, wherein there is a double bond between C2 and C3, and R² is a C₁₋₅ saturated aliphatic alkyl group.

15. A compound according to statement 14, wherein R² is methyl, ethyl or propyl.

16. A compound according to any one of statements 1 to 8, wherein there is a double bond between C2 and C3, and R² is a C₃₋₆ saturated cycloalkyl group.

17. A compound according to statement 16, wherein R² is cyclopropyl.

18. A compound according to any one of statements 1 to 8, wherein there is a double bond between C2 and C3, and R² is a group of formula:

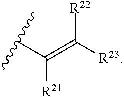

19. A compound according to statement 18, wherein the total number of carbon atoms in the R² group is no more than 4.

20. A compound according to statement 19, wherein the total number of carbon atoms in the R² group is no more than 3.

21. A compound according to any one of statements 18 to 20, wherein one of R²¹, R²² and R²³ is H, with the other two groups being selected from H, C₁₋₃ saturated alkyl, C₂₋₃ alkenyl, C₂₋₃ alkynyl and cyclopropyl.

22. A compound according to any one of statements 18 to 20, wherein two of R²¹, R²² and R²³ are H, with the other group being selected from H, C₁₋₃ saturated alkyl, C₂₋₃ alkenyl, C₂₋₃ alkynyl and cyclopropyl.

23. A compound according to any one of statements 1 to 8, wherein there is a double bond between C2 and C3, and R² is a group of formula:

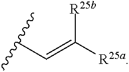

24. A compound according to statement 23, wherein R² is the group:

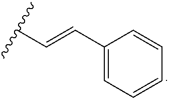 [SMILES: */C=C/c1ccccc1]

25. A compound according to any one of statements 1 to 8, wherein there is a double bond between C2 and C3, and R² is a group of formula:

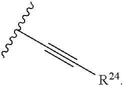

26. A compound according to statement 25, wherein R²⁴ is selected from H, methyl, ethyl, ethenyl and ethynyl.

27. A compound according to statement 26, wherein R²⁴ is selected from H and methyl.

28. A compound according to any one of statements 1 to 8, wherein there is a single bond between C2 and C3, R² is

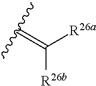

and R^{26a} and R^{26b} are both H.

29. A compound according to any one of statements 1 to 8, wherein there is a single bond between C2 and C3, R² is

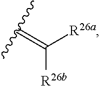

and R^{26a} and R^{26b} are both methyl.

30. A compound according to any one of statements 1 to 8, wherein there is a single bond between C2 and C3, R² is

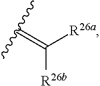

one of R^{26a} and R^{26b} is H, and the other is selected from C₁₋₄ saturated alkyl, C₂₋₃ alkenyl, which alkyl and alkenyl groups are optionally substituted.

31. A compound according to any one of statements 1 to 30, wherein there is a double bond between C2′ and C3′, and R¹² is a C₅₋₇ aryl group.

32. A compound according to statement 31, wherein R¹² is phenyl.

33. A compound according to any one of statements 1 to 30, wherein there is a double bond between C2′ and C3′, and R¹² is a C₈₋₁₀ aryl group.

34. A compound according to any one of statements 31 to 33, wherein R¹² bears one to three substituent groups.

35. A compound according to any one of statements 31 to 34, wherein the substituents are selected from methoxy, ethoxy, fluoro, chloro, cyano, bis-oxy-methylene, methyl-piperazinyl, morpholino and methyl-thiophenyl.

36. A compound according to any one of statements 1 to 30, wherein there is a double bond between C2′ and C3′, and R¹² is a C₁₋₅ saturated aliphatic alkyl group.

37. A compound according to statement 36, wherein R¹² is methyl, ethyl or propyl.

38. A compound according to any one of statements 1 to 30, wherein there is a double bond between C2′ and C3′, and R¹² is a C₃₋₆ saturated cycloalkyl group.

39. A compound according to statement 38, wherein R¹² is cyclopropyl.

40. A compound according to any one of statements 1 to 30, wherein there is a double bond between C2′ and C3′, and R¹² is a group of formula:

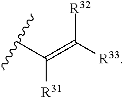

41. A compound according to statement 40, wherein the total number of carbon atoms in the R¹² group is no more than 4.

42. A compound according to statement 41, wherein the total number of carbon atoms in the R¹² group is no more than 3.

43. A compound according to any one of statements 40 to 42, wherein one of R³¹, R³² and R³³ is H, with the other two groups being selected from H, C₁₋₃ saturated alkyl, C₂₋₃ alkenyl, C₂₋₃ alkynyl and cyclopropyl.

44. A compound according to any one of statements 40 to 42, wherein two of R³¹, R³² and R³³ are H, with the other group being selected from H, C₁₋₃ saturated alkyl, C₂₋₃ alkenyl, C₂₋₃ alkynyl and cyclopropyl.

45. A compound according to any one of statements 1 to 30, wherein there is a double bond between C2′ and C3′, and R¹² is a group of formula:

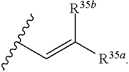

46. A compound according to statement 45, wherein R¹² is the group:

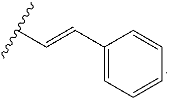 [SMILES: */C=C/c1ccccc1]

47. A compound according to any one of statements 1 to 30, wherein there is a double bond between C2′ and C3′, and R¹² is a group of formula:

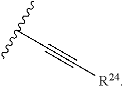

48. A compound according to statement 47, wherein R³⁴ is selected from H, methyl, ethyl, ethenyl and ethynyl.

49. A compound according to statement 48, wherein R³⁴ is selected from H and methyl.

50. A compound according to any one of statements 1 to 30, wherein there is a single bond between C2′ and C3′, R¹² is

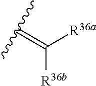

and R^{36a} and R^{36b} are both H.

51. A compound according to any one of statements 1 to 30, wherein there is a single bond between C2′ and C3′, R¹² is

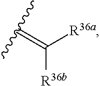

and R^{36a} and R^{36b} are both methyl.

52. A compound according to any one of statements 1 to 30, wherein there is a single bond between C2′ and C3′, R¹² is

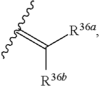

one of R^{36a} and R^{36b} is H, and the other is selected from C₁₋₄ saturated alkyl, C₂₋₃ alkenyl, which alkyl and alkenyl groups are optionally substituted.

53. A conjugate according to statement 1, wherein DL is:

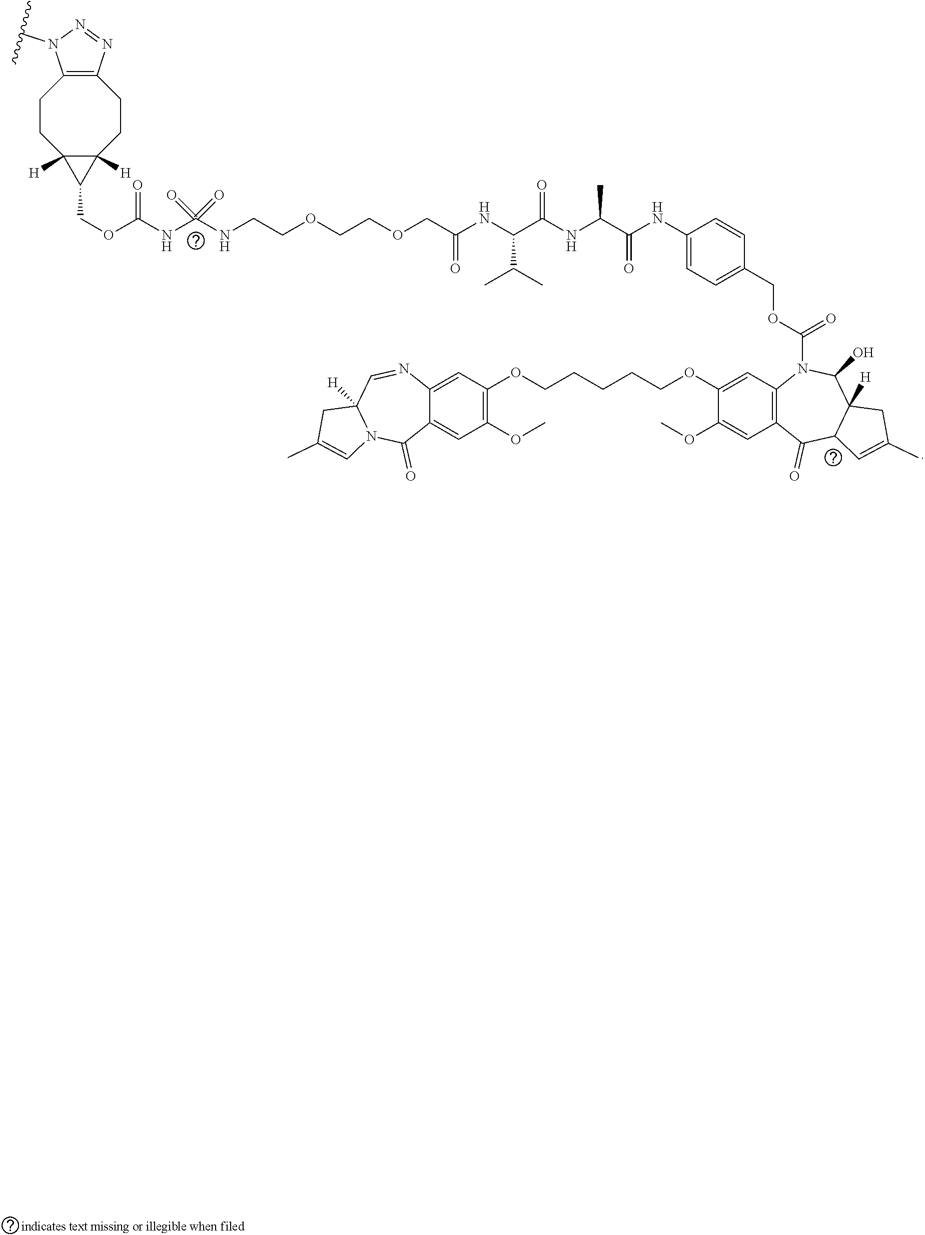

54. The conjugate according to any one of statements 1 to 53 wherein the cell binding agent (CBA) is an antibody (Ab).

The definition of the terms used in the above statements are as defined in WO2018/146199.

Conjugates having the structure shown in statement 53 above are particularly preferred.

### Cell Binding Agents (CBAs)

A cell binding agent may be of any kind, and include peptides and non-peptides. These can include antibodies or a fragment of an antibody that contains at least one binding site, lymphokines, hormones, hormone mimetics, vitamins, growth factors, nutrient-transport molecules, or any other cell binding molecule or substance.

### Antibodies

In preferred embodiments the cell-binding agent is an antibody.

The term “antibody” herein is used in the broadest sense and specifically covers monoclonal antibodies, polyclonal antibodies, dimers, multimers, multispecific antibodies {e.g., bispecific antibodies), and antibody fragments, so long as they exhibit the desired biological activity (Miller et al (2003) Jour, of Immunology 170:4854-4861). Antibodies may be murine, human, humanized, chimeric, or derived from other species. An antibody is a protein generated by the immune system that is capable of recognizing and binding to a specific antigen. (Janeway, C, Travers, P., Walport, M., Shlomchik (2001) ImmunoBiology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a fu II-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)2, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1 q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Techniques to reduce the in vivo immunogenicity of a non-human antibody or antibody fragment include those termed “humanisation”.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

### Preferred Antibodies

In some embodiments, the antibody binds CD25. In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589. The antibody may comprise a VH domain having a VH CDR1 with the amino acid sequence of SEQ ID NO.3, a VH CDR2 with the amino acid sequence of SEQ ID NO.4, and a VH CDR3 with the amino acid sequence of SEQ ID NO.5; for example the antibody may comprise a VH domain having the sequence according to SEQ ID NO. 1. The antibody may further comprise a VL domain having a VL CDR1 with the amino acid sequence of SEQ ID NO.6, a VL CDR2 with the amino acid sequence of SEQ ID NO.7, and a VL CDR3 with the amino acid sequence of SEQ ID NO.8; for example the antibody may comprise a VL domain having the sequence according to SEQ ID NO. 2.

In some embodiments, the antibody binds CD19. In some embodiments, CD19 polypeptide corresponds to Genbank accession no. NP_001171569, version no. NP_001171569.1 GI:296010921, record update date: Sep. 10, 2012 12:43 AM. In one embodiment, the nucleic acid encoding CD19 polypeptide corresponds to Genbank accession no NM_001178098, version no. NM_001178098.1 GI:296010920, record update date: Sep. 10, 2012 12:43 AM. In some embodiments, CD19 polypeptide corresponds to Uniprot/Swiss-Prot accession No. P15391. The antibody may comprise a VH domain having the sequence according to either one of SEQ ID Nos. 11 or 12, optionally further comprising a VL domain having the sequence according to any one of SEQ ID Nos. 13 or 14. The antibody may comprise VH and VL domains respectively having the sequences of: SEQ ID NO. 11 and SEQ ID NO. 13, or SEQ ID NO. 12 and SEQ ID NO. 14. In preferred embodiments the antibody comprises a VH domain having the sequence according to SEQ ID NO. 12. In preferred embodiments the antibody comprises a VL domain having the sequence according to SEQ ID NO. 14.

In some embodiments, the antibody binds CD22. In some embodiments, CD22 polypeptide corresponds to Genbank accession no. BAB15489, version no. BAB15489.1 GI:10439338, record update date: Sep. 11, 2006 11:24 PM. In one embodiment, the nucleic acid encoding CD22 polypeptide corresponds to Genbank accession no AK026467, version no. AK026467.1 GI:10439337, record update date: Sep. 11, 2006 11:24 PM. Preferably the antibody comprises a VH domain having the sequence according to SEQ ID NO. 15. Preferably the antibody comprises a VL domain having the sequence according to SEQ ID NO. 16. Most preferably the antibody comprises a heavy chain having the sequence according to SEQ ID NO. 17 and a light chain having the sequence according to SEQ ID NO. 18, optionally wherein the drug moiety is conjugated to the cysteine at position 219 of SEQ ID NO.17.

In some embodiments, the antibody binds PSMA. In one embodiment, PSMA polypeptide corresponds to Genbank accession no. AAA60209, version no. AAA60209.1 GI:190664, record update date: Jun. 23, 2010 08:48 AM. In one embodiment, the nucleic acid encoding PSMA polypeptide corresponds to Genbank accession no. M99487, version no. M99487.1 GI:190663, record update date: Jun. 23, 2010 08:48 AM. The antibody may comprise a VH domain having the sequence according to either one of SEQ ID Nos. 21 or 23. The antibody may further comprise a VL domain having the sequence according to either one of SEQ ID NOs. 22 or 24. Preferably the antibody comprises a VH domain having a sequence SEQ ID NO. 23 and a VL domain having a sequence SEQ ID NO. 24. Most preferably the antibody comprises: (a) a heavy chain having the sequence according to SEQ ID NO. 25, wherein the drug moiety is conjugated to the cysteine at position 218 of SEQ ID NO.25; (b) a light chain having the sequence according to SEQ ID NO. 26.

In some embodiments, the antibody binds AXL. In some embodiments, the AXL polypeptide corresponds to Genbank accession no. AAH32229, version no. AAH32229.1 GI:21619004, record update date: Mar. 6, 2012 01:18 PM. In one embodiment, the nucleic acid encoding AXL polypeptide corresponds to Genbank accession no. M76125, version no. M76125.1 GI:292869, record update date: Jun. 23, 2010 08:53 AM. In some embodiments the antibody comprises a VH domain having a VH CDR1 with the amino acid sequence of SEQ ID NO.35, a VH CDR2 with the amino acid sequence of SEQ ID NO.36, and a VH CDR3 with the amino acid sequence of SEQ ID NO.37. The antibody may further comprise a VL domain having a VL CDR1 with the amino acid sequence of SEQ ID NO.38, a VL CDR2 with the amino acid sequence of SEQ ID NO.39, and a VL CDR3 with the amino acid sequence of SEQ ID NO.40. In preferred embodiments the antibody comprises a VH domain having the sequence of SEQ ID NO.31 and a VL domain having the sequence of SEQ ID NO.32.

In some embodiments, the antibody binds DLK-1. In some embodiments, the DLK1 polypeptide corresponds to Genbank accession no. CAA78163, version no. CAA78163.1, record update date: Feb. 2, 2011 10:34 AM. In one embodiment, the nucleic acid encoding DLK1 polypeptide corresponds to Genbank accession no. Z12172, version no Z12172.1, record update date: Feb. 2, 2011 10:34 AM. The antibody may comprise a VH domain having a VH CDR1 with the amino acid sequence of SEQ ID NO.45, a VH CDR2 with the amino acid sequence of SEQ ID NO.46, and a VH CDR3 with the amino acid sequence of SEQ ID NO.47. The antibody may further comprise a VL domain having a VL CDR1 with the amino acid sequence of SEQ ID NO.48, a VL CDR2 with the amino acid sequence of SEQ ID NO.49, and a VL CDR3 with the amino acid sequence of SEQ ID NO.50. Preferably the antibody comprises a VH domain having the sequence of SEQ ID NO.41 and a VL domain having the sequence of SEQ ID NO.42. In some embodiments the antibody comprises a heavy chain having the sequence of SEQ ID NO. 43 or 51 paired with a light chain having the sequence of SEQ ID NO.44.

In some embodiments, the antibody binds KAAG1. In some embodiments, the KAAG1 polypeptide corresponds to Genbank accession no. AAF23613, version no. AAF23613.1. In one embodiment, the nucleic acid encoding KAAG1 polypeptide corresponds to Genbank accession no. AF181722, version no AF181722.1. The antibody may a VH domain having a VH CDR1 with the amino acid sequence of SEQ ID NO.65, a VH CDR2 with the amino acid sequence of SEQ ID NO.66, and a VH CDR3 with the amino acid sequence of SEQ ID NO.67. The antibody may further comprise a VL domain having a VL CDR1 with the amino acid sequence of SEQ ID NO.68, a VL CDR2 with the amino acid sequence of SEQ ID NO.69, and a VL CDR3 with the amino acid sequence of SEQ ID NO.70. In preferred embodiments the antibody comprises a VH domain having the sequence of SEQ ID NO.61 and a VL domain having the sequence of SEQ ID NO.62, SEQ ID NO.73, or SEQ ID NO.75. In some embodiments the antibody comprises a heavy chain having the sequence of SEQ ID NO. 63 or 71 and a light chain having the sequence of SEQ ID NO.64, SEQ ID NO.74, or SEQ ID NO.76.

In some embodiments, the antibody binds Mesothelin. In some embodiments, the Mesothelin polypeptide corresponds to Genbank accession no. AAC50348, version no. AAC50348.1, record update date: Jun. 23, 2010 09:12 AM. In one embodiment, the nucleic acid encoding Mesothelin polypeptide corresponds to Genbank accession no. U40434, version no U40434.1, record update date: Jun. 23, 2010 09:12 AM. The antibody may comprises a VH domain having the sequence of SEQ ID NO.81 and a VL domain comprises the sequence of SEQ ID NO.82. For example, the antibody may comprises a heavy chain having the sequence of SEQ ID NO. 83 or 91 and a light chain having the sequence of SEQ ID NO.84. The antibody may comprises a VH domain having the sequence of SEQ ID NO.92 and a VL domain comprises the sequence of SEQ ID NO.93. For example, the antibody may comprises a heavy chain having the sequence of SEQ ID NO. 94 or 102 and a light chain having the sequence of SEQ ID NO.95. The antibody may comprises a VH domain having the sequence of SEQ ID NO.103 and a VL domain comprises the sequence of SEQ ID NO.104. For example, the antibody may comprises a heavy chain having the sequence of SEQ ID NO. 105 or 113 and a light chain having the sequence of SEQ ID NO.106. The antibody may comprises a VH domain having the sequence of SEQ ID NO.114 and a VL domain comprises the sequence of SEQ ID NO.115. For example, the antibody may comprises a heavy chain having the sequence of SEQ ID NO. 116 or 118 and a light chain having the sequence of SEQ ID NO.117.

As used herein to describe antibodies, “binds [antigen]” (eg. “binds CD25”) means that the antibody binds the antigen with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds the antigen with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the disclosure can bind the antigen with a high affinity. For example, in some embodiments the antibody can bind the antigen with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

The antibody may be an intact antibody. The antibody may be humanised, deimmunised or resurfaced. The antibody may be a fully human monoclonal IgG1 antibody, preferably IgG1,κ.

### Preferred PBD Agents

### ADCx25

ADCx25 has the chemical structure:

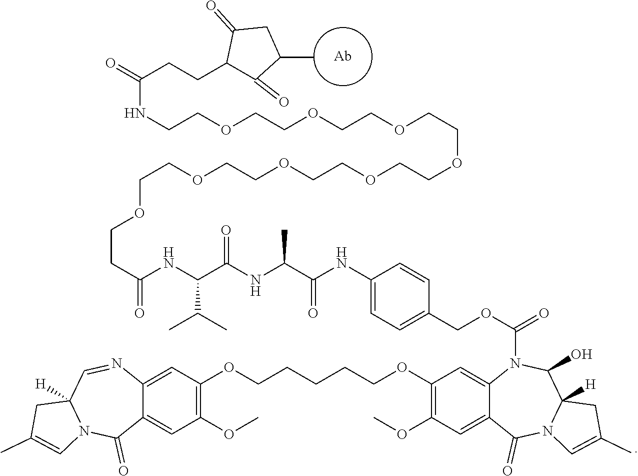

wherein “Ab” is the antibody AB12 (fully human monoclonal IgG1, K antibody with the VH and VL sequences SEQ ID NO. 1 and SEQ ID NO. 2, respectively, also known as HuMax-TAC). It is synthesised as described in W2014/057119 (Conj AB12-E) and typically has a DAR (Drug to Antibody Ratio) of 2.0+/−0.3.

### ADCx19

ADCx19 has the chemical structure shown above for ADCx25, except that in ADCx19 “Ab” represents Antibody RB4v1.2 (antibody with the VH and VL sequences SEQ ID NO. 12 and SEQ ID NO. 14, respectively). It is synthesised as described in WO2014/057117 (RB4v1.2-E) and typically has a DAR (Drug to Antibody Ratio) of 2+/−0.3.

### ADCx22

ADCx22 has the chemical structure shown above for ADCx25, except that in ADCx22 “Ab” represents Antibody EMabC220. This antibody comprises a heavy chain having the sequence according to SEQ ID NO. 17 and a light chain having the sequence according to SEQ ID NO. 18. Linkage to the drug occurs on Heavy Chain interchain cysteine Cys220 (EU numbering). HC220 corresponds to position 219 of SEQ ID NO.17.

It is noted that “having the sequence” has the same meaning as “comprising the sequence”; in particular, in some embodiments the heavy chain of ADCx22 is expressed with an additional terminal ‘K’ residue (so, ending . . . SPGK), with the terminal K being optionally removed post-translationally to improve the homogeneity of the final therapeutic ADC product.

### ADCxPSMA

ADCxPSMA has the chemical structure shown above for ADCx25, except that in ADCxPSMA “Ab” represents an antibody comprising:
(a) a heavy chain having the sequence according to SEQ ID NO. 25, wherein the drug moiety is conjugated to the cysteine at position 218 of SEQ ID NO.25;
(b) a light chain having the sequence according to SEQ ID NO. 26.

It is noted that “having the sequence” has the same meaning as “comprising the sequence”; in particular, in some embodiments the heavy chain of ADCxPSMA is expressed with an additional terminal ‘K’ residue (so, ending . . . SPGK), with the terminal K being optionally removed post-translationally to improve the homogeneity of the final therapeutic ADC product.

### ADCxAXL

ADCxAXL has the chemical structure:

Ab-(DL)ₚ

wherein:

### D^{L} is:

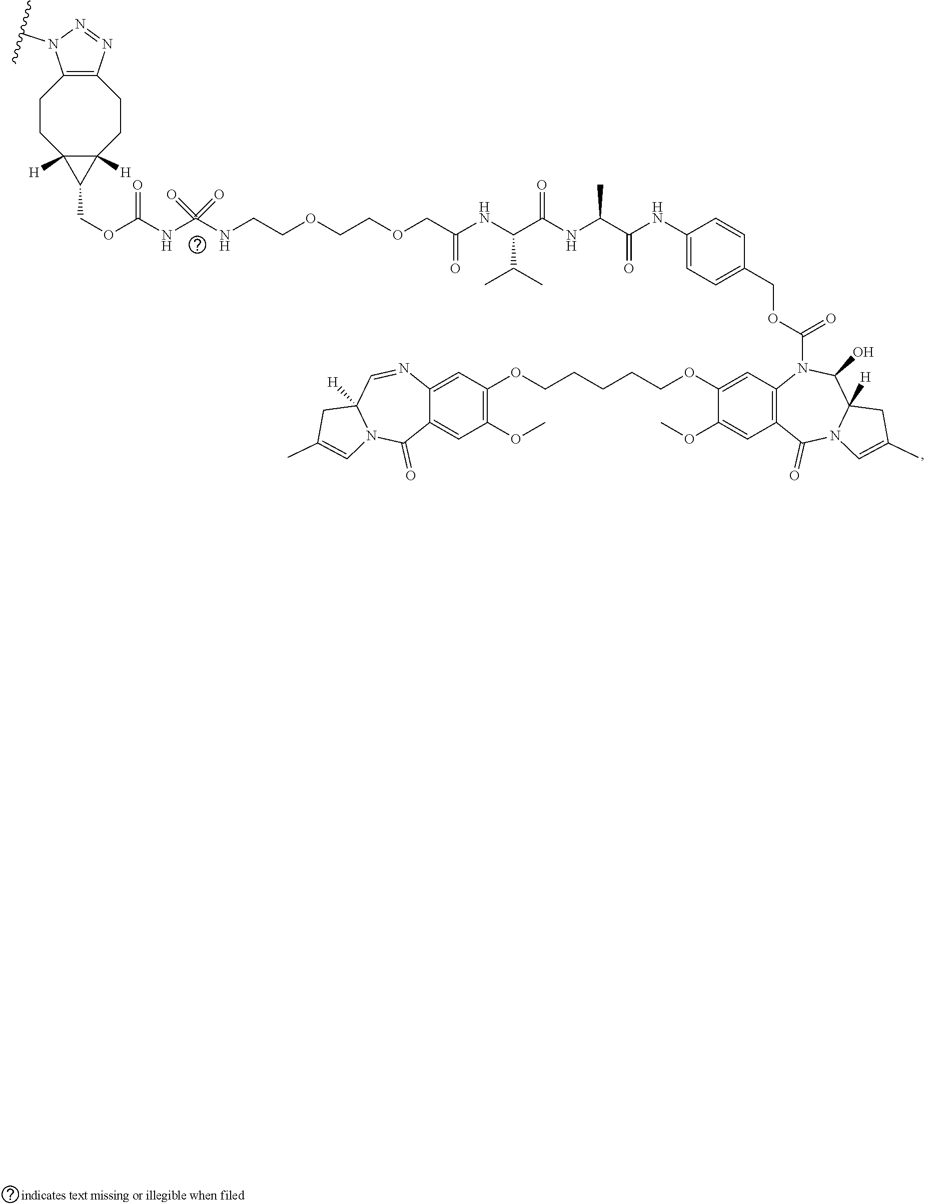

Ab is an antibody that binds to AXL, the antibody comprising:
(a) a heavy chain having the sequence according to SEQ ID NO. 33;
(b) a light chain having the sequence according to SEQ ID NO. 34.

It is noted that “having the sequence” has the same meaning as “comprising the sequence”; in particular, in some embodiments the heavy chain of ADCxAXL is expressed with an additional terminal ‘K’ residue (so, ending . . . SPGK), with the terminal K being optionally removed post-translationally to improve the homogeneity of the final therapeutic ADC product.

DL may be conjugated to the antibody through the sidechain of the asparagine at position 302 of SEQ ID NO.3. The structure of the linkage to the antibody may be N-[GlcNAc]-DL, wherein N is the asparagine residue, and [GlcNac] represents a GlcNAc residue. p may be up to 2, and is typically greater than 1.9.

### ADCxDLK1

ADCxDLK1 has the chemical structure shown above for ADCxAXL, except that in ADCxDLK1 “Ab” represents an antibody that binds to DLK1, the antibody comprising:
(a) a heavy chain having the sequence according to SEQ ID NO. 43;
(b) a light chain having the sequence according to SEQ ID NO. 44.

### ADCxKAAG1

ADCxKAAG1 has the chemical structure shown above for ADCxAXL, except that in ADCxKAAG1 “Ab” represents an antibody that binds to KAAG1, the antibody comprising:
(a) a heavy chain having the sequence according to SEQ ID NO. 63;
(b) a light chain having the sequence according to SEQ ID NO. 64, 74, or 76.

### ADCxMesothelin

ADCxMesothelin has the chemical structure shown above for ADCxAXL, except that in ADCxMesothelin “Ab” represents an antibody that binds to Mesothelin, the antibody comprising:
(a) a heavy chain having the sequence according to SEQ ID NO. 83 a light chain having the sequence according to SEQ ID NO. 84;
(b) a heavy chain having the sequence according to SEQ ID NO. 94 a light chain having the sequence according to SEQ ID NO. 95;
(c) a heavy chain having the sequence according to SEQ ID NO. 105 a light chain having the sequence according to SEQ ID NO. 106; or
(d) a heavy chain having the sequence according to SEQ ID NO. 116 a light chain having the sequence according to SEQ ID NO. 117.

In particularly preferred embodiments, the PBD agent is selected from ADCT-301, ADCT-401, ADCT-402, ADCT-602, ADCT-601, or ADCT-701.

### Treated Disorders

This disclosure describes methods for determining whether a proliferative disorder in a subject is resistant to treatment with a PBD agent. Related methods describe the selection of a subject for treatment with a PBD agent on the basis that the subject has a proliferative disorder that is not resistant to treatment with a PBD agent. Also described are methods for reducing the PBD resistance of a PBD-resistant proliferative disorder, allowing for the effective treatment of a PBD-resistant proliferative disorder using a PBD agent.

Certain aspects the therapies include a cell binding agent such as an antibody conjugated, i.e. covalently attached by a linker, to a PBD agent, i.e. toxin. When the agent is not conjugated to an antibody, the PBD compound has a cytotoxic effect. The biological activity of the PBD compound is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

The terms “proliferative disease” and “proliferative disorder” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders also include heamatological cancers including, but not limited to, non-Hodgkin's Lymphoma, including diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL), and leukemias such as Hairy cell leukemia (HCL), Hairy cell leukemia variant (HCL-v), and Acute Lymphoblastic Leukaemia (ALL) such as Philadelphia chromosome-positive ALL (Ph+ALL) or Philadelphia chromosome-negative ALL (Ph−ALL). [Fielding A., Haematologica. 2010 January; 95(1): 8-12].

The target proliferative cells may be all or part of a solid tumour.

“Solid tumor” herein will be understood to include solid haematological cancers such as lymphomas (Hodgkin's lymphoma or non-Hodgkin's lymphoma) which are discussed in more detail herein.

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the combined therapies may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, graft-versus-host disease (GVHD), and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

### Samples

The sample may comprise or may be derived from: a quantity of blood; a quantity of serum derived from the subject's blood which may comprise the fluid portion of the blood obtained after removal of the fibrin clot and blood cells; a quantity of pancreatic juice; a tissue sample or biopsy, in particular from a solid tumour; or cells isolated from said subject.

A sample may be taken from any tissue or bodily fluid. In certain aspects, the sample may include or may be derived from a tissue sample, biopsy, resection or isolated cells from said subject.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of CD25 polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In certain aspects, the sample is a fresh biopsy.

### Subjects

The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject may be any of its forms of development, for example, a foetus. In one preferred embodiment, the subject is a human. The terms “subject”, “patient” and “individual” are used interchangeably herein.

In some aspects the subject has, is suspected of having, has been diagnosed with, or is at risk of, a PBD-resistant proliferative disorder as described herein.

### Subject Selection

In certain aspects, the subjects are selected as suitable for treatment with the treatments before the treatments are administered. In some aspects the treatment methods described herein include the step of selecting suitable subjects. In some aspects the treatment methods described herein treat subjects that have been previously selected as suitable for treatment.

As used herein, subjects who are considered suitable for treatment are those subjects who are expected to benefit from, or respond to, the treatment. Subjects may have, be suspected of having, have been diagnosed with, or be at risk of, a disorder characterized by the presence of a proliferative cell, or cell population such as a tumour, that is resistant to treatment with a PBD-agent.

In some aspects the treated subject has been selected for treatment on the basis that the subject has, is suspected of having, has been diagnosed with, or is at risk of, a PBD-resistant disorder.

In some aspects the subject is: (1) selected for treatment on the basis that the subject has, is suspected of having, has been diagnosed with, or is at risk of, a PBD-resistant disorder; then (2) treated with a PBD agent as described herein.

In particular, the PBD-resistant disorder may be solid tumour as described herein.

In some aspects, subjects are selected on the basis of the amount or pattern of expression of one or more PBD-resistance genes. So, in some cases, subjects are selected on the basis they have, or are suspected of having, are at risk of having, or have received a diagnosis of a proliferative disease characterized by the amount or pattern of expression of PBD-resistance genes.

In some cases, expression of one or more PBD-resistance genes in a particular tissue of interest is determined. For example, in a sample of tumor tissue. In some cases, systemic expression of one or more PBD-resistance genes is determined. For example, in a sample of circulating fluid such as blood, plasma, serum or lymph.

In some aspects, the subject is selected as suitable for treatment with a PBD-agent due to the normal expression of one or more PBD-resistance genes in a sample. In those cases, subjects without normal expression (i.e with overexpression) of one or more PBD-resistance genes may be considered not suitable for treatment with a PBD-agent.

In some aspects, the subject is selected as suitable for treatment with an antagonist of a PBD-resistance gene due to the overexpression of one or more PBD-resistance genes in a sample. In those cases, subjects without over expression of one or more PBD-resistance genes may be considered not suitable for treatment with an antagonist of a PBD-resistance gene.

In other aspects, the level of PBD-resistance gene expression is used to select a subject as suitable for treatment. Where the level of expression of one or more PBD-resistance genes is below a threshold level, the subject is determined to be suitable for treatment with a PBD agent.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of a PBD agent. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the PBD agent alone (for example, to a subject that is, or has been determined to be, not resistant to treatment with PBD agents) or in further combination with other treatments, either simultaneously or sequentially dependent upon the condition to be treated.

Combination of a PBD agent with an antagonist of one or more PBD-resistance genes is a preferred embodiment (for example, to a subject that is, or has been determined to be, resistant to treatment with PBD agents). Such combination therapies noted herein encompass combined administration (where two or more therapeutic agents are included in the same or separate formulations), and separate administration, in which case, administration of the combined agent (eg. PBD-gene antagonist) can occur prior to, simultaneously, and/or following, administration of the PBD agent.

Examples of combined treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1l, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure, including vaccine compositions, are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Dosage

It will be appreciated by one of skill in the art that appropriate dosages of the PBD agent and compositions comprising this active element, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of PBD agent and/or PBD gene antagonist are determined by the expression of one or more PBD resistance genes observed in a sample obtained from the subject. Thus, the level or localisation of expression of PBD-resistance gene expression in the sample may be indicative that a higher or lower dose of PBD agent and/or PBD gene antagonist is required. For example, a high expression level of PBD-resistance gene may indicate that a higher dose of PBD agent and/or PBD gene antagonist would be suitable. In some cases, a high expression level of PBD-resistance gene may indicate the need for administration of another agent in addition to the PBD-agent, such as a PBD gene antagonist. A high expression level of PBD-resistance gene may indicate a more aggressive therapy.

In certain aspects, the dosage level is determined by the expression of CD25 on neoplastic cells in a sample obtained from the subject. For example, when the target neoplasm is composed of, or comprises, neoplastic cells expressing CD25

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the PBD agent the dosage amounts described above may apply to a conjugate (including a PBD moiety and the linker to the cell binding agency) or to the effective amount of PBD compound provided, for example the amount of compound that is releasable after cleavage of the linker.

### BRIEF DESCRIPTION OF THE FIGURES

Embodiments and experiments illustrating the principles of the disclosure will now be discussed with reference to the accompanying figures in which:

FIG. 1. 96-hour continuous exposure ADCT-301 cytotoxicity of Karpas wt and Karpas ADCR cells

FIG. 2. 96-hour continuous exposure SG3199 cytotoxicity of Karpas wt and Karpas PBDR cells

FIG. 3. 144-hour continuous exposure ADCT-502 cytotoxicity of NCI-N87 wt and NCI-N87 ADCR cells

FIG. 4. 144-hour continuous exposure SG3199 cytotoxicity of NCI-N87 wt and NCI-N87 PBDR cells

FIG. 5. 96-hour continuous exposure ADCT-301 cytotoxicity of Karpas wt and Karpas PBDR cells

FIG. 6. 96-hour continuous exposure ADCT-301 cytotoxicity of Karpas wt and Karpas PBDR cells

FIG. 7. 96-hour continuous exposure Humax-TAC-SG3560 cytotoxicity of Karpas wt, ADCR and PBDR cells.

FIG. 8. 144-hour continuous exposure SG3199 cytotoxicity of NCI-N87 wt, and ADCR cells

FIG. 9. 144-hour continuous exposure ADCT-502 cytotoxicity of NCI-N87 wt, and PBDR cells

FIG. 10. ICL formation of 130 pM ADCT-301 in Karpas wt, and ADCR cells

FIG. 11. ICL formation of 280 pM SG3199 in Karpas wt, and PBDR cells

FIG. 12. ICL formation of 1 nM ADCT-502 in NCI-N87 wt, and ADCR cells

FIG. 13. ICL formation of 1.7 nM SG3199 in NCI-N87 wt, and PBDR cells

FIG. 14. 2-hour exposure 130 pM ADCT-301 cytotoxicity of Karpas wt, and ADCR cells

FIG. 15. 2-hour exposure 280 pM SG3199 cytotoxicity of Karpas wt, and PBDR cells

FIG. 16. 2-hour exposure 1 nM ADCT-502 cytotoxicity of NCI-N87 wt, and ADCR cells

FIG. 17. 2-hour exposure 1.7 nM SG3199 cytotoxicity of NCI-N87 wt, and PBDR cells

FIG. 18. ICL formation of 280 pM SG3199 in Karpas wt, and ADCR cells

FIG. 19. ICL formation of 130 pM ADCT-301 in Karpas wt, and PBDR cells

FIG. 20. ICL formation of 1.7 nM SG3199 in NCI-N87 wt, and ADCR cells

FIG. 21. ICL formation of 1 nM ADCT-502 in NCI-N87 wt, and PBDR cells

FIG. 22. 2-hour exposure 280 pM SG3199 cytotoxicity of Karpas wt, and ADCR cells

FIG. 23. 2-hour exposure 130 pM ADCT-301 cytotoxicity of Karpas wt, and PBDR cells

FIG. 24. 2-hour exposure 1 nM SG3199 cytotoxicity of NCI-N87 wt & ADCR cells

FIG. 25. 2-hour exposure 1.7 nM ADCT-502 cytotoxicity of NCI-N87 wt, and PBDR cells

FIG. 26. Karpas wt and resistant cell line binding flow cytometry

FIG. 27. Volcano plot of Karpas wt vs ADCR drug transporter gene expression

FIG. 28. Volcano plot of Karpas wt vs PBDR drug transporter gene expression

FIG. 29. Volcano plot of NCI-N87 wt vs ADCR drug transporter gene expression

FIG. 30. Volcano plot of NCI-N87 wt vs PBDR drug transporter gene expression

FIG. 31. Karpas ADCR and PBDR drug transporter gene expression compared to wt cells

FIG. 32. NCI-N87 ADCR and PBDR drug transporter gene expression compared to wt cells

FIG. 33. Western blot of Karpas wt and resistant cell lines probed for ABCC2 and ABCG2

FIG. 34. Western blot of NCI-N87 wt and resistant cell lines probed for ABCC2 and ABCG2

FIG. 35. Karpas wt and resistant cell line CD25 antibody internalisation

FIG. 36. NCI-N87 wt and resistant cell line HER2 antibody internalisation

FIG. 37. 96-hour continuous exposure MK-571 and ADCT-301 cytotoxicity of Karpas wt and Karpas ADCR cells

FIG. 38. 96-hour continuous exposure MK-571 and ADCT-301 cytotoxicity of Karpas wt and Karpas PBDR cells

FIG. 39. NCI-N87 wt and resistant cell line binding flow cytometry

FIG. 40. Reversing the acquired PBD or PBD-based ADC resistance with ABC drug transporter inhibitors: continuous exposure in vitro growth inhibition of Karpas-299 and NCI-N87 wt, ADC and PBD resistant cell lines with target ADC and either 5 μM MK-571 or 10 μM FTC

FIG. 41. Reversing the acquired PBD or PBD-based ADC resistance with ABC drug transporter inhibitors: Continuous exposure in vitro growth inhibition of Karpas-299 and NCI-N87 wt, ADC and PBD resistant cell lines with SG3199 and either 5 μM MK-571 or 10 μM FTC

FIG. 42. Reversing the acquired PBD or PBD-based ADC resistance with ABC drug transporter inhibitors:
A. Continuous exposure in vitro growth inhibition of Karpas-299 wt, ADC and PBD resistant cell lines with ADCT-301 and 10 nM Lovastatin.
B. Continuous exposure in vitro growth inhibition of NCI-N87 wt, ADC and PBD resistant cell lines with ADCT-502 and 5 μM Lovastatin. Each data point represents the average of at least 3 biological repeats with +/−SD error bars.

FIG. 43. Reversing the acquired PBD or PBD-based ADC resistance with ABC drug transporter inhibitors: Interstrand cross-link formation in Karpas-299 and NCI-N87 wt, ADC and PBD resistant cell lines with target ADC or SG3199 (Karpas, 130 μM ADCT-301; 280 μM SG3199, NCI-N97, 1 nM ADCT-502; 1.7 nM SG3199) and either 5 μM MK-571 or 10 μM FTC

FIG. 44. Reversing the acquired PBD or PBD-based ADC resistance by ABCC2 siRNA knockdown in NCI-N87 resistant cells: A. Representative western blot for ABCC2 and ABCG2 in Karpas-299 and NCI-N87 wt, ADC and PBD resistant cell lines. B. Representative western blot for ABCC2 NCI-N87 ADC and PBD resistant cell lines with siRNA against ABCC2 or scramble control.

FIG. 45. Reversing the acquired PBD or PBD-based ADC resistance by ABCC2 siRNA knockdown in NCI-N87 resistant cells: ABCC2 siRNA knockdown continuous exposure in vitro growth inhibition of NCI-N87 wt, ADC and PBD resistant cell lines with ADCT-502 or SG3199. Each data point represents the average of at least 3 biological repeats with +/−SD error bars.

### STATEMENTS OF INVENTION

1. A method for determining whether a proliferative disease in a subject is resistant to treatment with a pyrrolobenzodiazepine (PBD) agent,
the method comprising determining whether one or more PBD-resistance genes are overexpressed in a sample from the subject,
wherein overexpression of the one or more PBD-resistance genes indicates that the proliferative disease is resistant to treatment with the PBD agent.

2. A method for selecting a subject for treatment with a pyrrolobenzodiazepine (PBD) agent, the method comprising the steps of:
(a) determining whether one or more PBD-resistance genes are overexpressed in a sample from the subject, and
(b) selecting the subject for treatment with the PBD agent if overexpression of the one or more PBD-resistance genes is not detected in the sample.

3. The method of statement 2, wherein the subject has, is suspected of having, has been diagnosed with, or is at risk of, a proliferative disease.

4. A method of reducing the resistance of a proliferative cell to a PBD-agent, the method comprising contacting the proliferative cell with an antagonist of one or more PBD-resistance genes.

5. A method of treating a proliferative disease in a subject, wherein the proliferative disease is resistant to a PBD agent, the method comprising administering to the subject an antagonist of one or more PBD-resistance genes in combination with a therapeutically effective amount of the PBD agent.

6. The method of either one of statements 4 or 5, wherein the antagonist is administered before the PBD agent, simultaneous with the PBD agent, or after the PBD agent.

7. The method of any one of statements 4 to 6, wherein the antagonist reduces the level of mRNA transcription from the one or more PBD-resistance genes.

8. The method of any one of statements 4 to 7, wherein the antagonist reduces the level of one or more PBD-resistance polypeptide expression.

9. The method of any one of statements 4 to 8, wherein the antagonist reduces the activity of one or more PBD-resistance polypeptide.

10. The method of any one of statements 4 to 9, wherein the antagonist is selected from the group consisting of:
(a) an anti-PBD-resistance antibody;
(b) an RNA agent that reduces the expression of one or more PBD-resistance gene;
(c) a mimetic or analog of (b);
and
(d) an agent that increased the expression of the RNA agent of (b).

11. The method of statement 10, wherein the antagonist is miR-200c, miR-212, miR-328, miR-519c, miR-520h, miR-297, or miR-379.

12. The method of any one of statements 4 to 9, wherein the antagonist is selected from the group consisting of: MK-571, Biricodar, Probenecid, Reversan, Fumitremorgin C, and Ko143.

13. The method of any one of statements 4 to 10, wherein the one or more PBD-resistance genes comprises ABCC2 and the antagonist reduces ABCC2 activity.

14. The method of statement 13, wherein the antagonist is an ABCC2 inhibitor selected from the group consisting of: probenecid, furosemide, ritonavir, saquinavir, lamivudine, abacavir, emtricitabine, efavirenz, delavirdine, nevirapine, cidofovir, adefovir, tenofovir, cyclosporine, PSC833, and MK571.

15. The method of any one of statements 4 to 10, wherein the antagonist reduces ABCC2 expression.

16. The method of statement 15, wherein the antagonist is an siRNA, or an miRNA, such as miR-297 or miR-379.

17. The method of any one of statements 4 to 10, wherein the one or more PBD-resistance genes comprises ABCG2 and the antagonist reduces ABCG2 activity.

18. The method of statement 17, wherein the antagonist is an ABCG2 inhibitor selected from the group consisting of: febuxostat, Fumitremorgin C, elacridar, tariquidar, and Ko143.

19. The method of any one of statements 4 to 10, wherein the antagonist reduces ABCG2 expression.

20. The method of statement 19, wherein the antagonist is an siRNA, or an miRNA, such as miR-200c, miR-212, miR-328, miR-519c, or miR-520h.

21. A method of treating a proliferative disease in a subject, the method comprising
a) selecting a subject for treatment with a pyrrolobenzodiazepine (PBD) agent according to either one of statements 2 or 3, and
b) administering to the subject a therapeutically effective amount of the PBD agent, optionally wherein the PBD agent is administered according to the method of any one of statements 5 to 20.

22. The method of any one of statements 1 to 21, wherein the one or more PBD-resistance genes are selected from the group consisting of: ABCG2, ABCC2, SLCO2B1, SLC7A7, SLC22A3, SLCO2A1, ABCC12, ATP7A, AQP7, SLC5A1, SLC16A2, SLC7A9, ABCB4, ABCC11, ABCF1, SLC28A3, and ABCB6.

23. The method of any one of statements 1 to 22, wherein the one or more PBD-resistance genes are selected from the group consisting of: ABCG2, ABCC2, SLCO2B1, SLC7A7, SLC22A3, SLCO2A1, ABCC12, ATP7A, AQP7, and SLC5A1.

24. The method of any one of statements 1 to 23, wherein the one or more PBD-resistance genes are selected from the group consisting of: ABCG2, ABCC2, SLCO2B1, SLC7A7, and SLC22A3.

25. The method of any one of statements 1 to 24, wherein the one or more PBD-resistance genes are selected from the group consisting of: ABCG2, ABCC2, SLCO2B1, and SLC7A7.

26. The method of any one of statements 1 to 25, wherein the one or more PBD-resistance genes are selected from the group consisting of: ABCG2, ABCC2, and SLCO2B1.

27. The method of any one of statements 1 to 25, wherein the one or more PBD-resistance genes are selected from the group consisting of: ABCG2, ABCC2, and SLC7A7.

28. The method of any one of statements 1 to 25, wherein the one or more PBD-resistance genes are selected from the group consisting of: ABCG2, ABCC2, and SLC22A3.

29. The method of any one of statements 1 to 28, wherein the one or more PBD-resistance genes are selected from the group consisting of: ABCG2 and ABCC2.

30. The method of any one of statements 1 to 26, wherein the one or more PBD-resistance genes are selected from the group consisting of: ABCG2 and SLCO2B1.

31. The method of any one of statements 1 to 30, wherein the one or more PBD-resistance genes are selected from the group consisting of: ABCG2.

32. The method of any one of statements 1 to 29, wherein the one or more PBD-resistance genes are selected from the group consisting of: ABCC2.

33. The method of any one of statements 1 to 30, wherein two or more PBD-resistance genes are selected.

34. The method of any one of statements 1 to 28, wherein three or more PBD-resistance genes are selected.

35. The method of any one of statements 1 to 25, wherein four or more PBD-resistance genes are selected.

36. The method of any one of statements 1 to 24, wherein five or more PBD-resistance genes are selected.

37. The method of any one of statements 1 to 36, determining PBD-resistance gene overexpression comprises measuring the level of mRNA transcription from the one or more PBD-resistance genes.

38. The method of statement 37, wherein the level of mRNA transcription is determined by cDNA PCR array, RT-PCR, fluorescence in situ hybridization (FISH), Southern Blot, immunohistochemisty (IHC), polymerase chain reaction (PCR), quantitative PCR (qPCR), quantitative real-time PCR (qRT-PCR), comparative genomic hybridization, microarray based comparative genomic hybridization, or ligase chain reaction (LCR).

39. The method of any one of statements 1 to 36, wherein determining PBD-resistance gene overexpression comprises measuring the level of PBD-resistance polypeptide expression.

40. The method of statement 39, wherein determining the level of PBD-resistance polypeptide expression comprises contacting the sample with an anti-PBD-resistance antibody and detecting binding of the anti-PBD-resistance antibody to PBD-resistance polypeptide.

41. The method of any one of statements 1 to 36, wherein determining PBD-resistance gene overexpression comprises measuring PBD-resistance polypeptide activity.

42. The method of any one of statements 1 to 41, wherein overexpression is indicated by an at least 2-fold increase relative to a control sample.

43. The method of statement 42, wherein overexpression is indicated by an at least 5-fold increase relative to a control sample.

44. The method of statement 43, wherein overexpression is indicated by an at least 10-fold increase relative to a control sample.

The method of statement 44, wherein overexpression is indicated by an at least 20-fold increase relative to a control sample.

46. The method of statement 45, wherein overexpression is indicated by an at least 50-fold increase relative to a control sample.

47. The method of statement 46 wherein overexpression is indicated by an at least 100-fold increase relative to a control sample.

48. The method of any one of statements 1 to 47, wherein overexpression is indicated by an increase relative to a control sample that has a p-value no greater than 0.05.

49. The method of any one of statements 1 to 48, wherein overexpression is indicated by an increase relative to a control sample that has a p-value no greater than 0.01.

50. The method of any one of statements 1 to 49, wherein overexpression is indicated by an increase relative to a control sample that has a p-value no greater than 0.005.

51. The method of any one of statements 1 to 50, wherein overexpression is indicated by an increase relative to a control sample that has a p-value no greater than 0.001.

52. The method of any one of statements 1 to 51, wherein overexpression is indicated by an increase relative to a control sample that has a p-value no greater than 0.0005.

53. The method of any one of statements 1 to 52, wherein overexpression is indicated by an increase relative to a control sample that has a p-value no greater than 0.0001.

54. The method of any one of statements 1 to 53, wherein overexpression is indicated by an increase relative to a control sample that has a p-value no greater than 0.00005.

55. The method of any one of statements 1 to 54, wherein overexpression is indicated by an increase relative to a control sample that has a p-value no greater than 0.00001.

56. The method of any one of statements 1 to 55, the proliferative disease is cancer.

57. The method of any one of statements 1 to 56, wherein the proliferative disorder or cancer is a benign, pre malignant, or malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis.

58. The method of any one of statements 1 to 56, wherein the proliferative disorder or cancer is a solid tumour.

59. The method of statement 58, wherein the solid tumour is associated with CD25+ve infiltrating cells;
optionally wherein the solid tumour is associated with high levels of CD25+ve infiltrating cells.

60. The method of statement 59, wherein the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

61. The method of any one of statements 1 to 56, wherein the proliferative disorder or cancer is lymphoma or leukaemia.

62. The method of statement 61, wherein the proliferative disorder or cancer is selected from:
Hodgkin's Lymphoma;
non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and
leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

63. The method of any one of statements 1 to 62, wherein the sample is a neoplasm sample.

64. The method of any one of statements 1 to 63, wherein the sample is a tumour sample.

65. The method of any one of statements 1 to 64, wherein the sample is a circulating fluid such as blood, plasma, serum or lymph.

66. The method of any one of statements 1 to 65, wherein the control is the expression level of the one or more PBD-resistance genes in a healthy sample from the subject.

67. The method of any one of statements 1 to 66, wherein the control is the expression level of the one or more PBD-resistance genes in a healthy subject.

68. The method of any one of statements 1 to 65, wherein the control is the expression level of the one or more PBD-resistance genes in a subject having a disorder known not to be PBD-resistant.

69. The method of statement 68, wherein the subject having a disorder that is known not to be PBD-resistant has previously been successfully treated with a PBD agent.

70. The method of any one of statements 1 to 65, wherein the control is the average expression level of the one or more PBD-resistance genes in a control population.

71. The method of statement 70, wherein the control population is a population of healthy subjects.

72. The method of any one of statements 1 to 71, wherein the sample and control are taken from the same tissue.

73. The method of any one of statements 1 to 72, wherein the PBD agent comprises a compound of the formula:

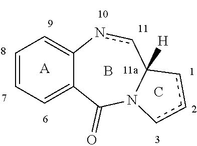

wherein positions 1-3 and 6-9 are optionally substituted;
wherein the B-ring has either an imine (N═C), a carbinolamine (NH—CH(OH)), or a carbinolamine methyl ether (NH—CH(OMe)) at the N10-C11 position; and, optionally
wherein the chiral C11a position has a (S)-configuration.

74. The method of any one of statements 1 to 73, wherein the PBD agent is, comprises, or releases a compound of the formula:

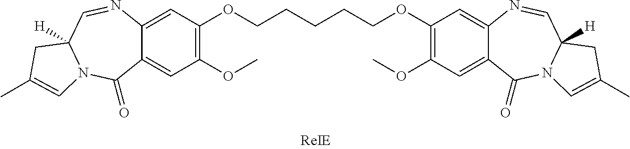

75. The method of any one of statements 1 to 74, wherein the PBD agent is a conjugate of formula L-(D^{L})ₚ, where D^{L} is of formula I or II:

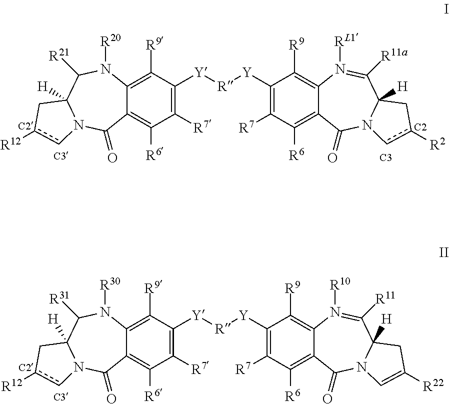

wherein:
L is a cell binding agent (CBA);
when there is a double bond present between C2′ and C3′, R¹² is selected from the group consisting of:
   (ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
   (ib) C₁₋₅ saturated aliphatic alkyl;
   (ic) C₃₋₆ saturated cycloalkyl;

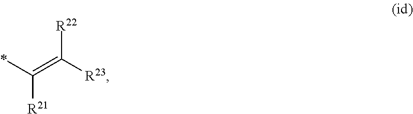

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

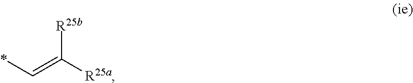

wherein one of R^{25a} and R^{25b} is H and the other is selected from: phenyl, which phenyl is optionally substituted by a group selected from halo, methyl, methoxy; pyridyl; and thiophenyl; and

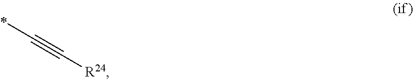

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

### R¹² is

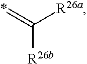

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from 0, S, or NH;
R^{6′}, R^{7′}, R^{9′} are selected from the same groups as R⁶, R⁷ and R⁹ respectively;

### [Formula I]

R^{L1′} is a linker for connection to the cell binding agent (CBA);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

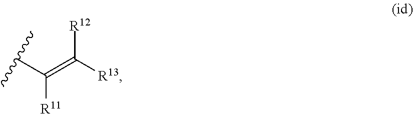

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

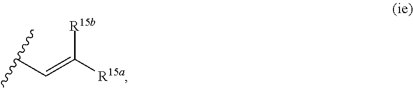

wherein one of R^{15a} and R^{15b} is H and the other is selected from: phenyl, which phenyl is optionally substituted by a group selected from halo, methyl, methoxy; pyridyl; and thiophenyl; and

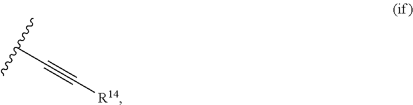

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

### R² is

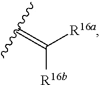

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

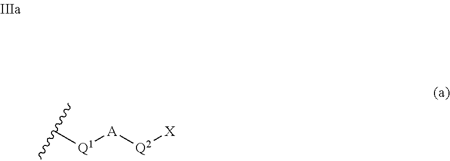

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

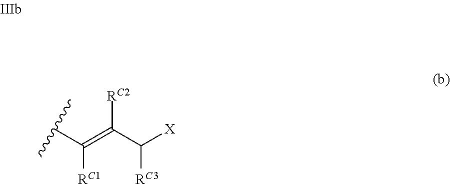

where;
R^{C1}, R^{C2} and R^{C3} are independently selected from H and unsubstituted C₁₋₂ alkyl;

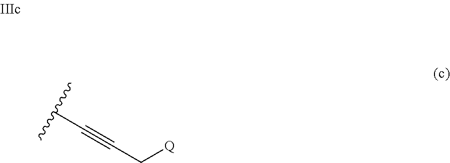

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, NH—C(═O)—R^{L2′}, NHNH—R^{L2′}, CONHNH—R^{L2′},

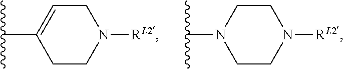

NR^{N}R^{L2′}, wherein R^{N} is selected from the group comprising H and C₁₋₄ alkyl;
R^{L2′} is a linker for connection to the cell binding agent (CBA);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

76. The method of statement 75, wherein the PBD agent comprises a compound of the formula:

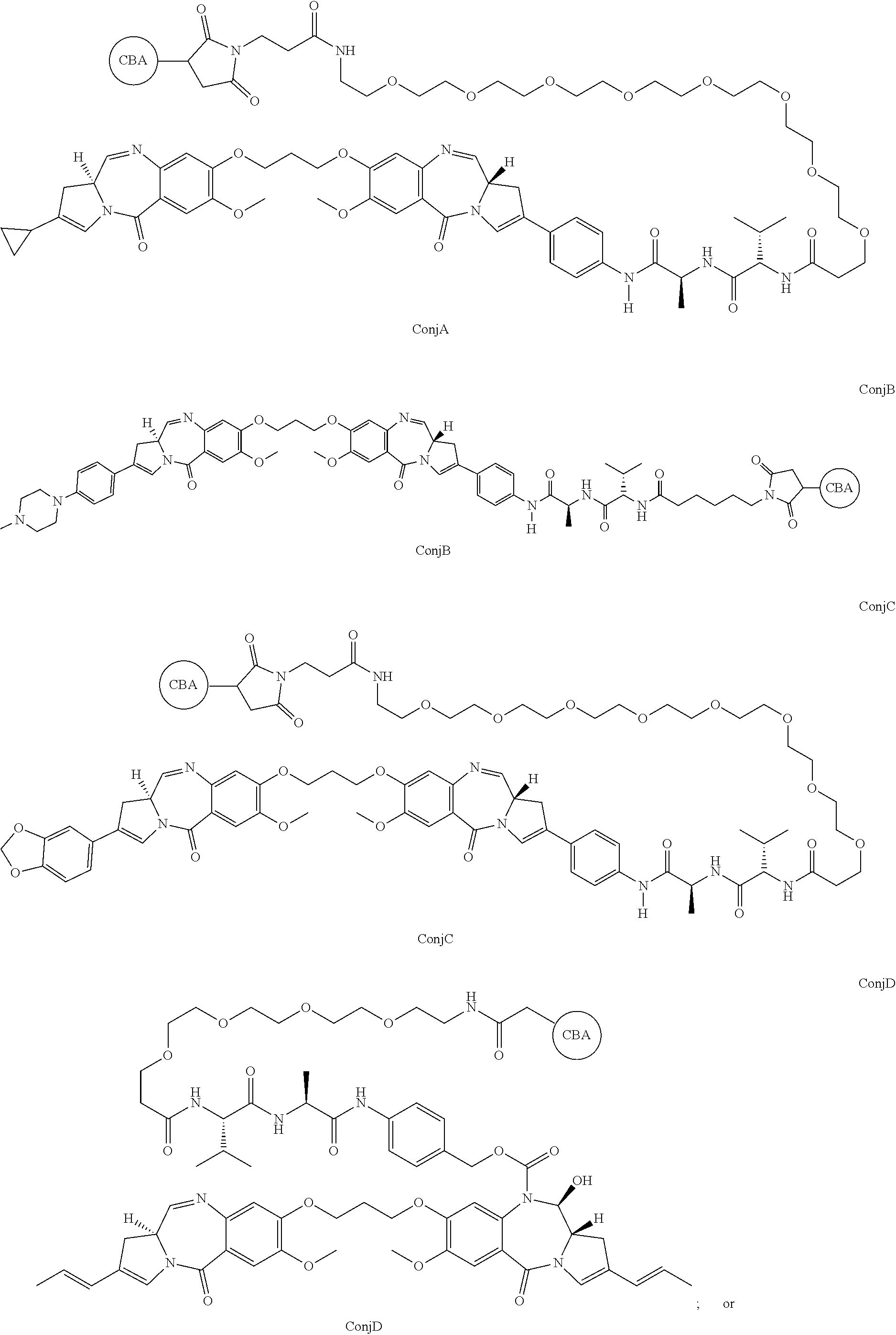

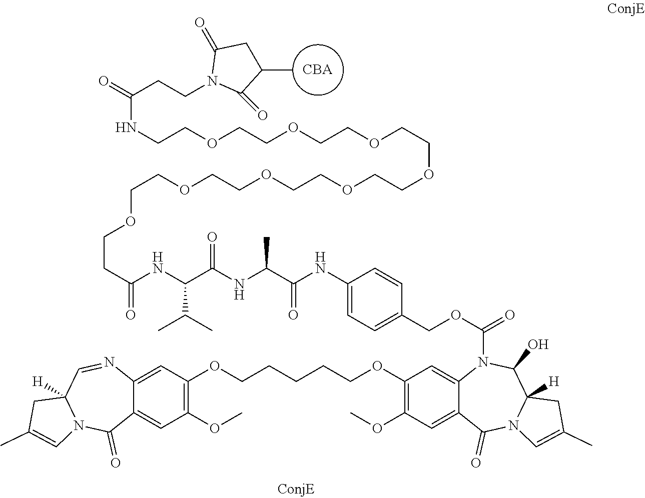

77. The method of any one of statements 1 to 73, wherein the PBD agent is a compound of the formula (III):

L-(DL)ₚ (Ill)

wherein:
L is a cell binding agent (CBA);

### DL is

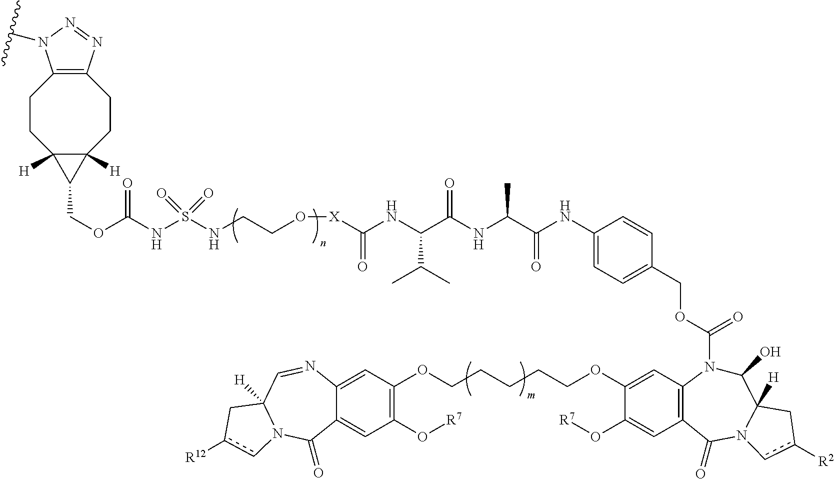

wherein:
X is selected from the group comprising: a single bond, —CH₂— and —C₂H₄—;
n is from 1 to 8;
m is 0 or 1;
R⁷ is either methyl or phenyl;
when there is a double bond between C2 and C3, R² is selected the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C3-6 saturated cycloalkyl;

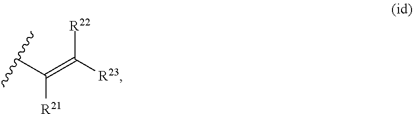

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R² group is no more than 5;

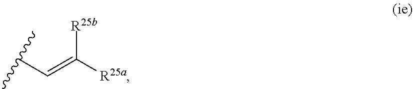

wherein one of R^{25a} and R^{25b} is H and the other is selected from: phenyl, which phenyl is optionally substituted by a group selected from halo, methyl, methoxy; pyridyl; and thiophenyl; and

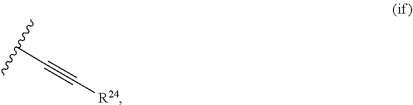

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2 and C3, R² is

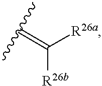

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
when there is a double bond between C2′ and C3′, R¹² is selected the group consisting of:
(iia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

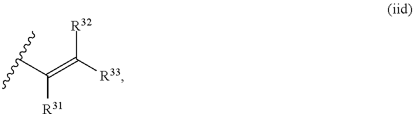

wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R¹² group is no more than 5;

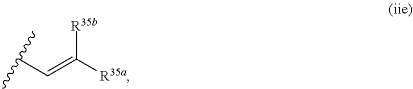

wherein one of R^{35a} and R^{35b} is H and the other is selected from: phenyl, which phenyl is optionally substituted by a group selected from halo, methyl, methoxy; pyridyl; and thiophenyl; and

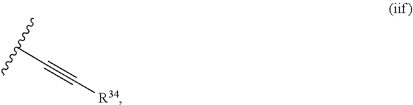

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2′ and C3′, R¹² is

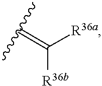

where R^{36a} and R^{36b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{36a} and R^{36b} is H, the other is selected from nitrile and a C₁₋₄ alkyl ester;
and p is from 1 to 8.

78. The method of statement 77, wherein DL is:

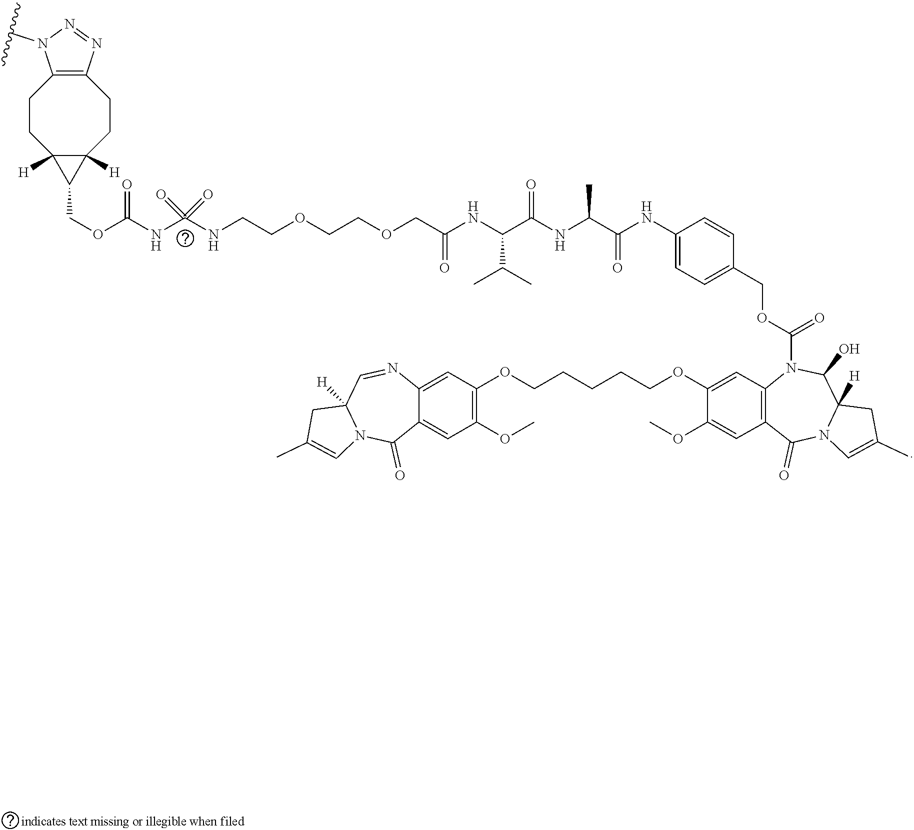

79. The method of any one of statements 1 to 75, wherein the PBD agent comprises a PBD conjugated to a cell binding agent.

80. The method of any one of statements 1 to 76, wherein the cell-binding moiety is an antibody.

81. The method of statement 80, wherein the antibody binds CD25 and comprises a VH domain having the sequence of SEQ ID NO.1 and a VL domain having the sequence of SEQ ID NO.2.

82. The method of statement 80, wherein the antibody binds CD19 and comprises a VH domain having the sequence of SEQ ID NO.12 and a VL domain having the sequence of SEQ ID NO.14.

83. The method of statement 80, wherein the antibody binds CD22 and comprises a VH domain having the sequence of SEQ ID NO.15 and a VL domain having the sequence of SEQ ID NO.16.

84. The method of statement 80, wherein the antibody binds PSMA and comprises a VH domain having the sequence of SEQ ID NO.23 and a VL domain having the sequence of SEQ ID NO.24.

85. The method of statement 80, wherein the antibody binds AXL and comprises a VH domain having the sequence of SEQ ID NO.31 and a VL domain having the sequence of SEQ ID NO.32.

86. The method of statement 80, wherein the antibody binds DLK1 and comprises a VH domain having the sequence of SEQ ID NO.41 and a VL domain having the sequence of SEQ ID NO.42.

87. The method of statement 80, wherein the antibody binds KAAG1 and comprises a VH domain having the sequence of SEQ ID NO.61 and a VL domain having the sequence of SEQ ID NO.62, SEQ ID NO.73, or SEQ ID NO.75.

88. The method of statement 80, wherein the antibody binds Mesothelin and comprises:
(a) a VH having the sequence of SEQ ID NO. 81 a VL having the sequence of SEQ ID NO. 82;
(b) a VH having the sequence of SEQ ID NO. 92 a VL having the sequence of SEQ ID NO. 93;
(c) a VH having the sequence of SEQ ID NO. 103 a VL having the sequence of SEQ ID NO. 104; or
(d) a VH having the sequence of SEQ ID NO. 114 a VL having the sequence of SEQ ID NO. 115.

89. The method of any one of statements 1 to 73, wherein the PBD agent is ADCx25.

90. The method of any one of statements 1 to 73, wherein the PBD agent is ADCx19.

91. The method of any one of statements 1 to 73, wherein the PBD agent is ADCx22.

92. The method of any one of statements 1 to 73, wherein the PBD agent is ADCxPSMA.

93. The method of any one of statements 1 to 73 wherein the PBD agent is ADCxAXL.

94. The method of any one of statements 1 to 73, wherein the PBD agent is ADCxDLK1.

95. The method of any one of statements 1 to 73, wherein the PBD agent is ADCxKAAG1.

96. The method of any one of statements 1 to 73, wherein the PBD agent is ADCxMesothelin.

97. The method of any one of statements 1 to 73, wherein the PBD agent is selected from ADCT-301, ADCT-401, ADCT-402, ADCT-602, ADCT-601, or ADCT-701.

98. A PBD agent as defined in any one of statements 73 to 97 for use in a method of any one of statements 5 to 72.

99. Use of a PBD agent as defined in any one of statements 73 to 97 in the preparation of a medicament for use in a method of any one of statements 5 to 72.

100. An antagonist of one or more PBD-resistance genes as defined in any one of statements 4 to 20 for use in a method of any one of statements 4 to 97.

101. Use of an antagonist of one or more PBD-resistance genes as defined in any one of statements 4 to 20 in the preparation of a medicament for use in a method of any one of statements 4 to 97.

### EXAMPLES

### Example 1: ADC and PBD Acquired Resistance Cell Line Generation

### Generation of Acquired Resistance in Human Haematological Karpas-299 Cell Line

To generate a resistant population to either ADCT-301 or SG3199, human anaplastic large cell lymphoma Karpas-299 cells (expressing CD25) were incubated with an approximate IC50 dose of the drug for 96 hours. The cells were then washed and returned to fresh medium until normal cell growth was recovered. This process was repeated until a measurable and stable loss in cytotoxic efficacy was established using the MTS assay.

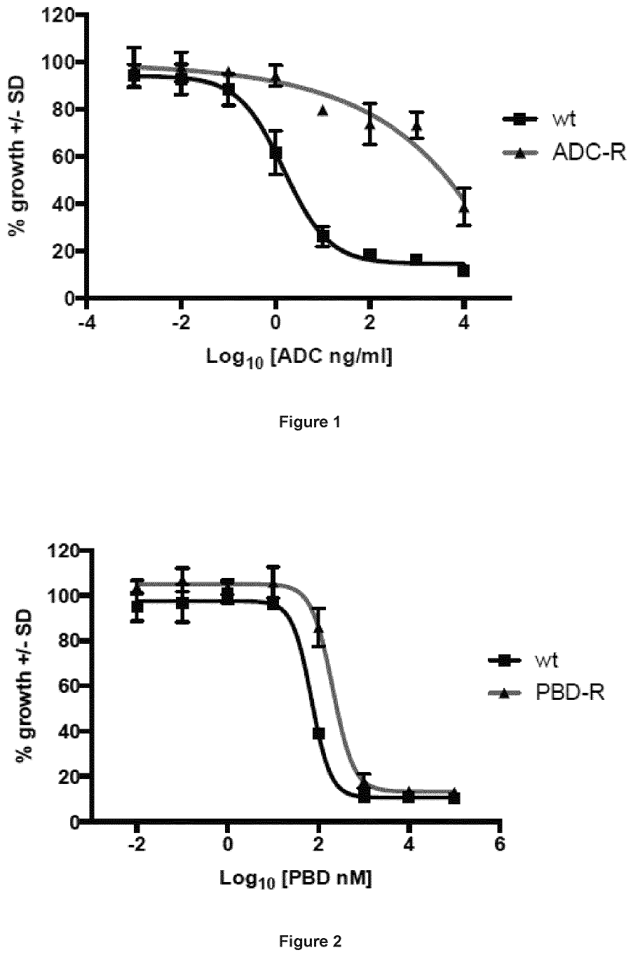

To measure the in vitro cytotoxicity with the MTS assay, cells were incubated with a 10-fold dilution range of ADCT-301 or SG3199, and incubated with continuous exposure for 96 hours (at least three cell doubling times under normal cell culture conditions). After incubation the CellTiter 960 AQueous One Solution Cell Proliferation Assay (MTS) was used to measure growth inhibition, by adding 20 μl reagent to the cells and incubating for 3-4 hours, then reading the absorbance at 492 nm. Mean % growth of the ADC or PBD treated cells was calculated relative to an untreated control. The growth inhibition curves for the wildtype and ADCT-301 acquired resistant cells are shown in FIG. 1, and for SG3199 resistant cells in FIG. 2.

### Generation of Acquired Resistance in Human Solid Tumour NCI-N87 Cell Line

To generate a resistant population to either ADCT-502 or SG3199, human gastric cancer NCI-N87 cells (expressing HER2) were incubated with an approximate IC50 dose of the drug for 144 hours. The cells were then washed and returned to fresh medium until normal cell growth was recovered. This process was repeated until a measurable and stable loss in cytotoxic efficacy was established using the MTS assay.

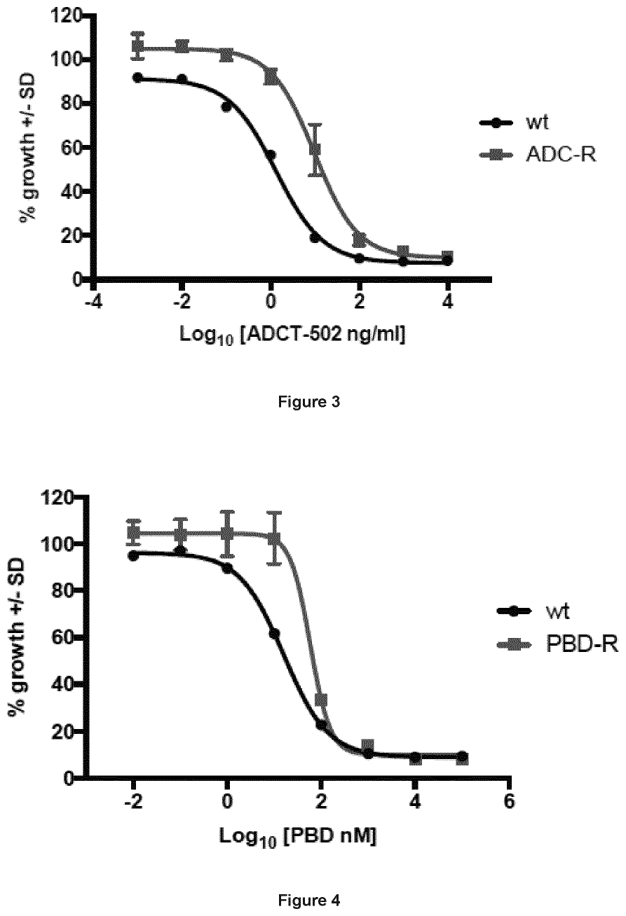

To measure the in vitro cytotoxicity with the MTS assay, cells were incubated with a 10-fold dilution range of ADCT-502 or SG3199, and incubated with continuous exposure for 144 hours (at least three cell doubling times under normal cell culture conditions). After incubation the CellTiter 960 AQueous One Solution Cell Proliferation Assay (MTS) was used to measure growth inhibition, by adding 20 μl reagent to the cells and incubating for 3-4 hours, then reading the absorbance at 492 nm. Mean % growth of the ADC or PBD treated cells was calculated relative to an untreated control. The growth inhibition curves for the wildtype and ADCT-502 acquired resistant cells are shown in FIG. 3, and for SG3199 resistant cells in FIG. 4.

### Example 2: Cross Resistance

### Determination of Cross-Resistance in Karpas-299 Acquired Resistant Cell Lines

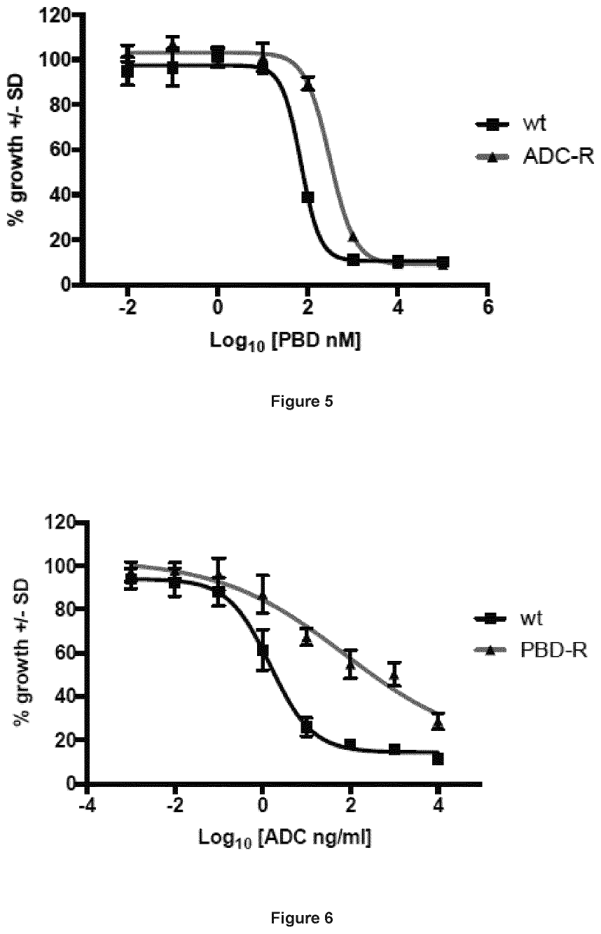

To measure the cross-resistance of the Karpas ADCR cells to the SG3199 PBD warhead, and the PBDR cells to ADCT-301, the in vitro cytotoxicity assay was carried out as described above, treating the cells with the opposite drug molecule to the one with which they were generated. The growth inhibition curves for the wildtype and ADCT-301 acquired resistant cells are shown in FIG. 5, and for SG3199 resistant cells in FIG. 6.

The mean IC50 values and fold resistance compared to the wt cell line are detailed in the table below.

| | ADCT-301 IC₅₀ | SG3199 IC₅₀ pM |
|---|---|---|
| | ng/mL (fold | (fold resistance |
| | resistance compared | compared to |
| Cell line | to wildtype) | wildtype) |
| Karpas wt | 1.5 | 1.5 |
| Karpas ADCr | >1000 (>667) | 306.5 (4.4) |
| Karpas PBDr | 72.8 (48.5) | 204.5 (2.9) |

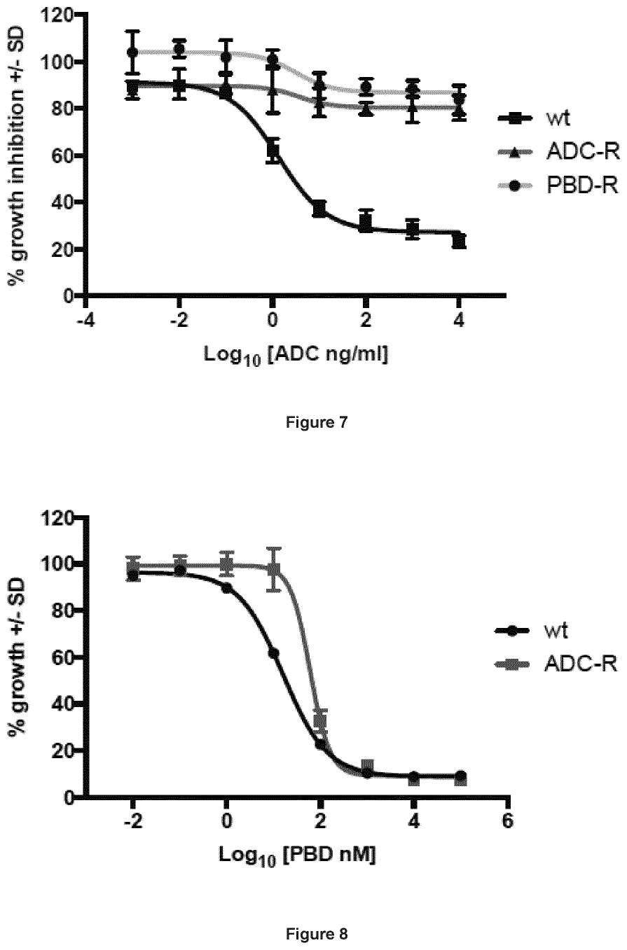

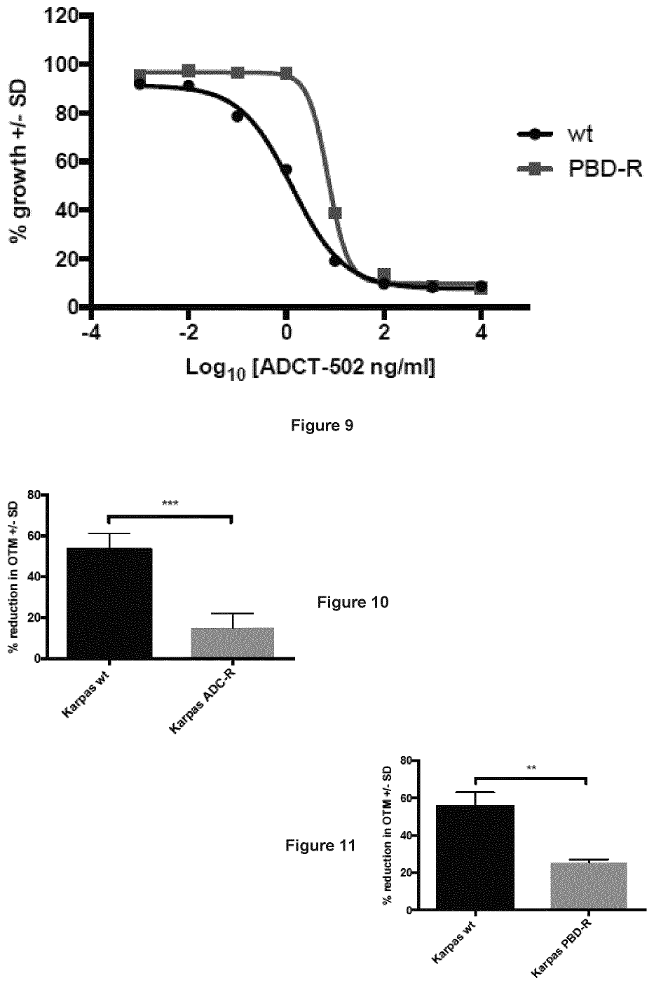

To further investigate the cross-resistance of the Karpas ADCR and PBDR cells to other PBD dimer-containing ADCs, cytotoxic sensitivity of both resistant cell lines was compared to the wt cells treated with the C2-linked PBD-ADC HuMax-TAC-SG3560, using the same MTS assay protocol previously described (FIG. 7). Cross-resistance was clearly demonstrated in both acquired resistant cell lines.

### Determination of Cross-Resistance in NCI-N87 Acquired Resistant Cell Lines

To measure the cross-resistance of the NCI-N87 ADCR cells to the SG3199 PBD warhead, and the PBDR cells to ADCT-502, the in vitro cytotoxicity assay was carried out as described above, treating the cells with the opposite drug molecule to the one with which they were generated. The mean IC50 values and fold resistance compared to the wt cell line are detailed in the table below.

The mean IC50 values and fold resistance compared to the wt cell line are detailed in the table below.

| | ADCT-502 IC₅₀ | SG3199 IC₅₀ pM |
|---|---|---|
| | ng/mL (fold | (fold resistance |
| | resistance compared | compared |
| Cell line | to wildtype) | to wildtype) |
| NCI-N87 wt | 1.3 | 15.9 |
| NCI-N87 ADCr | 9.9 (7.6) | 60.4 (3.8) |
| NCI-N87 PBDr | 7.2 (5.5) | 58.6 (3.7) |

### Example 3: DNA Interstrand Cross-Linking

### Determination of DNA Interstrand Cross-Linking in Karpas-299 Wild Type and Acquired Resistant Cell Lines

The formation of inter-strand cross-links (ICLs) by either ADCT-301 or SG3199 was measured using a modification of the single cell gel electrophoresis (comet) assay (Spanswick V J, Hartley J M, Hartley J A. Measurement of DNA interstrand crosslinking in individual cells using the Single Cell Gel Electrophoresis (Comet) assay. Methods Mol Biol. 613, 267-82 (2010)). Cells were treated with 130 μM ADCT-301 or 280 μM SG3199 for 2 hours, then washed and incubated for 24 hours under normal cell culture conditions. All cells were irradiated with 18 Gy (5 Gy/min for 3.6 min). Comet slides were reviewed under a 20× objective on an epifluorescence microscope equipped with: Hg arc lamp; 580 nm dichroic mirror; and 535 nm excitation and 645 nm emission filters suitable for visualising of propidium iodide staining with a minimum of 50 Comet images acquired per treatment condition. The Olive Tail Moment (OTM) was determined as the product of the tail length and the fraction of total DNA in the tail as recorded by Komet 6 software (Andor Technology, Belfast, UK) and the percentage reduction in OTM calculated according to the formula:

% decrease in tail moment=[1−(TMdi−TMcu)/(TMci−TMcu)]*100
[TMdi=Tail Moment drug irradiated; TMcu=TM control un-irradiated; TMci=TM control irradiated]

The level of DNA interstrand cross-linking for ADCT-301 in Karpas-ADCR and for SG3199 in Karpas-PBDR compared to wildtype cells are shown in FIGS. 10 and 11, respectively. The decreased cross-linking indicates an ‘upstream’ mechanism of acquired resistance, i.e. a mechanism that prevents the drug from reaching its ultimate target (DNA).

### Determination of DNA Interstrand Cross-Linking in NCI-N87 Wild Type and Acquired Resistant Cell Lines

The formation of inter-strand cross-links (ICLs) by either ADCT-502 or SG3199 was measured using the modification of the single cell gel electrophoresis (comet) assay. Cells were treated with 1 nM ADCT-502 or 1.7 nM SG3199 for 2 hours, then washed and incubated for 24 hours under normal cell culture conditions. All cells were irradiated with 18 Gy (5 Gy/min for 3.6 min). ICL formation was quantitated by measuring Olive tail moment (OTM) using the Komet 4 software, and the percentage reduction in OTM compared to an untreated irradiated control.

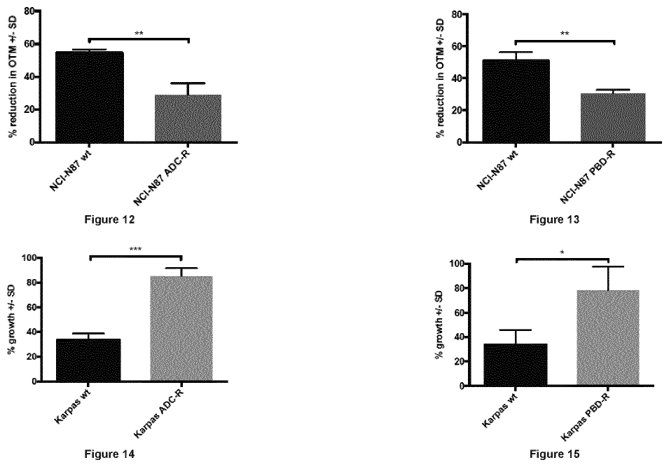

The level of DNA interstrand cross-linking for ADCT-502 in NCI-N87-ADCR and for SG3199 in NCI-N87-PBDR compared to wildtype cells are shown in FIGS. 12 and 13, respectively.

### Karpas-299 2-Hour Exposure Cytotoxicity

To investigate growth inhibition under the same conditions under which DNA ICL formation was measured, Karpas-ADCR cells were treated with 130 pM ADCT-301, and Karpas-PBDR cells treated with 280 pM SG3199 for 2 hours, then washed and incubated for 96 hours under normal cell culture conditions. The MTS assay and calculation of % growth compared to the untreated control was carried out as previously described.

The resulting growth inhibition values for ADCT-301 in Karpas-ADCR versus wildtype and for SG3199 in Karpas-PBDR versus wildtype are shown in FIGS. 14 and 15, respectively.

### NCI-N87 2-Hour Exposure Cytotoxicity

To investigate growth inhibition under the same conditions under which DNA ICL formation was measured, NCI-N87-ADCR cells were treated with 1 nM ADCT-502, and PBDR cells treated with 1.7 nM SG3199 for 2 hours, then washed and incubated for 144 hours under normal cell culture conditions. The MTS assay and calculation of % growth compared to the untreated control was carried out as previously described.

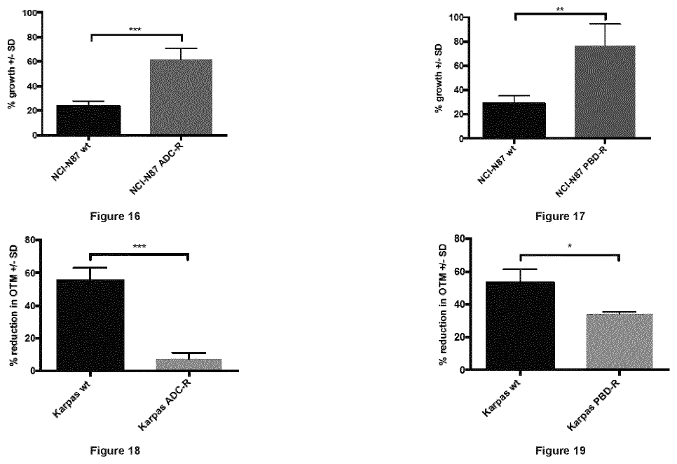

The resulting growth inhibition values for ADCT-502 in NCI-N87-ADCR versus wildtype and for SG3199 in NCI-N87-PBDR versus wildtype are shown in FIGS. 16 and 17, respectively.

### Karpas-R Cross-Resistant ICL Formation

To investigate if the cross-resistance of the Karpas ADCR cells to the SG3199 PBD warhead, and the PBDR cells to ADCT-301, also shows a change in ICL formation, the comet assay was carried out as described above, treating the cells with the opposite drug molecule to the one with which they were generated. The results are shown in FIG. 18 for the ADCR line and FIG. 19 for the PBDR line.

The mean % reduction in OTM and p-value of the t-tests comparing the resistant line with the wt cell line are detailed in the table below.

| | ADCT-301 % red. | SG3199 % red. |
|---|---|---|
| Cell line | OTM (p-value) | OTM (p-value) |
| Karpas wt | 53.5 | 55.5 |
| Karpas ADCr | 15 (0.0004) | 7.2 (0.0006) |
| Karpas PBDr | 34.3 (0.01) | 25.7 (0.003) |

### NCI-N87 Cross-Resistant ICL Formation

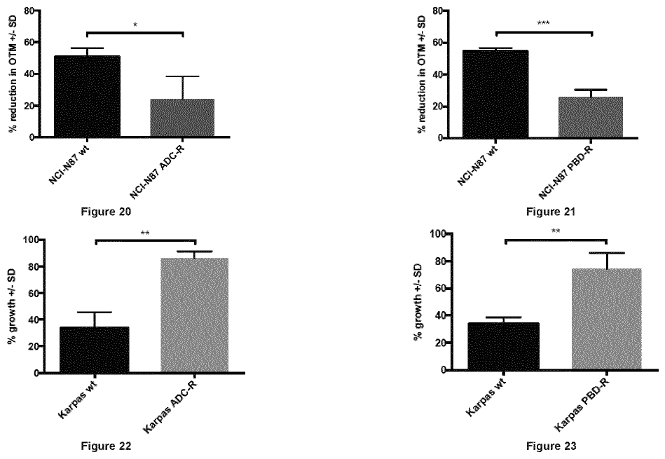

To investigate if the cross-resistance of the NCI-N87 ADCR cells to the SG3199 PBD warhead, and the PBDR cells to ADCT-502, also shows a change in ICL formation, the comet assay was carried out as described above, treating the cells with the opposite drug molecule to the one with which they were generated. The results are shown in FIG. 20 for the ADCR line and FIG. 21 for the PBDR line.

The mean % reduction in OTM and p-value of the t-tests comparing the resistant line with the wt cell line are detailed in the table below.

| | ADCT-502 % red. | SG3199 % red. |
|---|---|---|
| Cell line | OTM (p-value) | OTM (p-value) |
| NCI-N87 wt | 54.7 | 51.0 |
| NCI-N87 ADCr | 29 (0.004) | 24 (0.04) |
| NCI-N87 PBDr | 26 (0.0005) | 30.7 (0.004) |

### Karpas Cross-Resistance Single Dose 2-Hour Exposure Cytotox

To investigate whether the cross-resistance in ICL formation observed in the Karpas resistant cell lines also correlates with growth inhibition at this dose, ADCR cells were treated with 280 μM SG3199, and PBDR cells treated with 130 μM ADCT-301 for 2 hours, then washed and incubated for 96 hours under normal cell culture conditions. The MTS assay and calculation of % growth compared to the untreated control was carried out as previously described. The results are shown in FIG. 22 for the ADCR line and FIG. 23 for the PBDR line.

The mean % growth values and p-values from the t-test comparing the resistant cells to the wt cell line are detailed in the table below.

| | ADCT-301 2 | SG3199 2 |
|---|---|---|
| | hr mean % | hr mean % |
| Cell line | growth (p-value) | growth (p-value) |
| Karpas wt | 33.8 | 33.8 |
| Karpas ADCr | 85.9 (0.0004) | 86.3 (0.008) |
| Karpas PBDr | 74.7 (0.01) | 78.5 (0.035) |

### NCI-N87 Cross-Resistant Single-Dose Cytotox

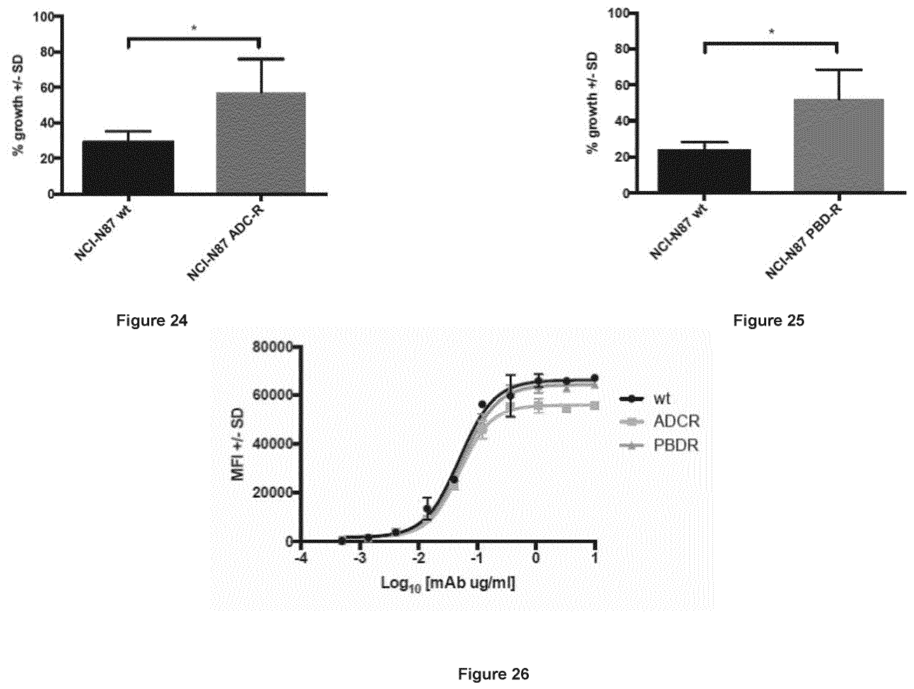

To investigate whether the cross-resistance in ICL formation observed in the NCI-N87 resistant cell lines also correlates with growth inhibition at this dose, ADCR cells were treated with 1 nM SG3199, and PBDR cells treated with 1.7 nM ADCT-502 for 2 hours, then washed and incubated for 144 hours under normal cell culture conditions. The MTS assay and calculation of % growth compared to the untreated control was carried out as previously described. The results are shown in FIG. 24 for the ADCR line and FIG. 25 for the PBDR line.

The mean % growth values and p-values from the t-test comparing the resistant cells to the wt cell line are detailed in the table below.

| | ADCT-502 2 | SG3199 2 |
|---|---|---|
| | hr mean % | hr mean % |
| Cell line | growth (p-value) | growth (p-value) |
| NCI-N87 wt | 23.5 | 29.0 |
| NCI-N87 ADCr | 61.5 (0.0008) | 57.2 (0.03) |
| NCI-N87 PBDr | 51.9 (0.02) | 76.6 (0.004) |

### Cross-Resistance to Other Chemotherapeutic Drugs

Cross-resistance to three conventional DNA-interacting chemotherapeutic drugs, cisplatin, doxorubicin and melphalan was also measured using the MTS assay in Karpas and NCI-N87 parental, ADC and PBD resistant cell lines. No cross-resistance was seen between any of these drugs in either the Karpas-299 ADCr or PBDr cell lines. In contrast, there was a decrease in growth inhibition in both NCI-N87 ADCr and PBDr cells treated with all three of these drugs compared to the parental cell line.

### Example 4: Antigen Expression

### Karpas Resistant Line CD25 Antibody Binding

Karpas wt and acquired resistant cells were blocked at 4° C. for 30 mins before being incubated with a serial dilution series of HuMax-TAC mAb for 1 hour on ice. The cells were then washed and incubated with a F(ab′)2-Goat anti-Human IgG Fc Secondary Antibody conjugated to Alexa Fluor 488. After incubation for 1 hour on ice in the dark the cells were washed and run on a Fortessa X20 flow cytometer, and the mean fluorescence intensity (MFI) determined using the B530/30 laser/filter. The resulting MFI curves are shown in FIG. 26. A small decrease in peak MFI was observed in the Karpas-299 ADCR cells, whereas the PBDR cells were identical to wildtype cells. In NCI-N87 cell lines there was a small decrease in MFI reached by the PBDr cell line, but the ADCr cell line was unchanged compared to the parental line (data not shown). However all cells continued to express high levels of antigen.

### Example 5: Human Drug Transporter Expression—PCR Array

### Karpas-R Drug Transporters PCR Array

The real-time RT2 Profiler PCR Array for human drug-transporters (Qiagen) was used to probe cDNA generated from Karpas acquired resistant and wt cell line lysates. CT values were normalized based on a panel of housekeeping genes (HKG). The Qiagen data analysis web portal calculates fold change/regulation using ΔΔCT method, in which ΔCT was calculated between gene of interest (GOI) and an average of HKGs, followed by DD CT calculations (ΔCT(Test Group)−ΔCT(Control Group)). Fold Change was then calculated using 2\hat{ }(−ΔΔCT) formula.

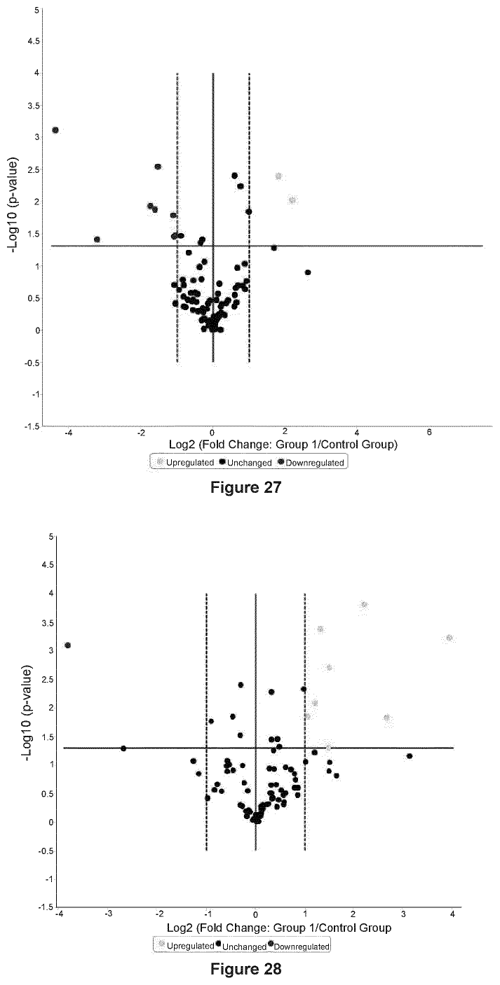

The volcano plot of Karpas wildtype versus Karpas-ADCR is shown in FIG. 27. The fold upregulation and p-value for the significantly upregulated genes in the acquired resistant line are shown in the table.

| Gene | Fold upregulation | (p-value) |
|---|---|---|
| ABCG2 | 168.1 | 0.0002 |
| SLCO2A1 | 4.6 | 0.009 |
| ABCC2 | 3.5 | 0.004 |

The volcano plot of Karpas wildtype versus Karpas-PBDR is shown in FIG. 28. The fold upregulation and p-value for the significantly upregulated genes in the acquired resistant line are shown in the table.

| Gene | Fold upregulation | (p-value) |
|---|---|---|
| SLCO2B1 | 15.5 | 0.0006 |
| ABCC12 | 6.4 | 0.01 |
| ATP7A | 4.6 | 0.0001 |
| SLC16A2 | 2.8 | 0.002 |
| ABCG2 | 2.8 | 0.05 |
| SLC7A9 | 2.5 | 0.0004 |
| ABCB4 | 2.3 | 0.008 |
| ABCC11 | 2.1 | 0.01 |

### NCI-N87-R Drug Transporters PCR Array

The real-time RT2 Profiler PCR Array for human drug-transporters (Qiagen) was used to probe cDNA generated from NCI-N87 resistant and wt cell line lysates. CT values were normalized based on a panel of housekeeping genes (HKG). The Qiagen data analysis web portal calculates fold change/regulation using ΔΔCT method, in which ΔCT was calculated between gene of interest (GOI) and an average of HKGs, followed by DD CT calculations (ΔCT(Test Group)−ΔCT(Control Group)). Fold Change was then calculated using 2\hat{ }(−ΔΔCT) formula.

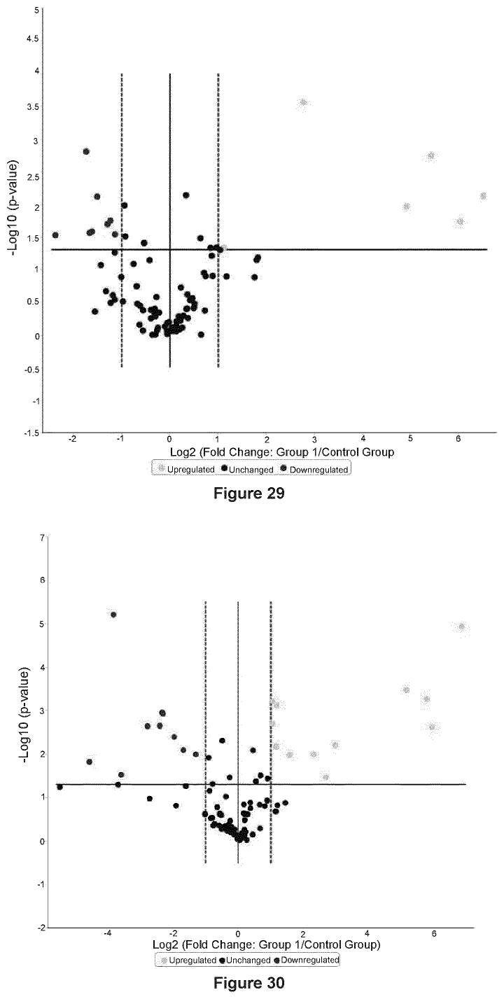

The volcano plot of NCI-N87 wildtype versus NCI-N87-ADCR is shown in FIG. 29. The fold upregulation and p-value for the significantly upregulated genes in the acquired resistant line are shown in the table.

| Gene | Fold upregulation | (p-value) |
|---|---|---|
| ABCG2 | 91.0 | 0.007 |
| SLC22A3 | 65.3 | 0.02 |
| ABCC2 | 43.0 | 0.002 |
| SLCO2B1 | 30.3 | 0.01 |
| SLC7A7 | 6.8 | 0.0003 |
| ABCF1 | 2.2 | 0.05 |

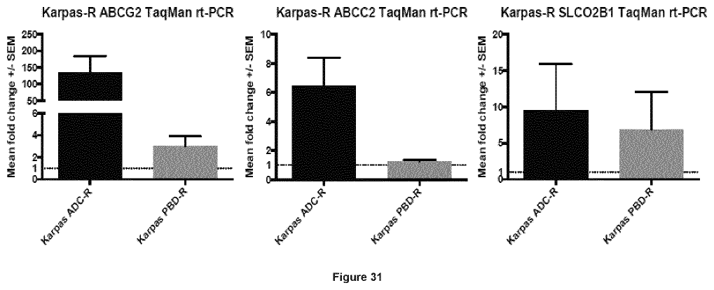

The volcano plot of NCI-N87 wildtype versus NCI-N87-PBDR is shown in FIG. 30. The fold upregulation and p-value for the significantly upregulated genes in the acquired resistant line are shown in the table.

| Gene | Fold upregulation | (p-value) |
|---|---|---|
| ABCG2 | 114.5 | 0.00001 |
| SLC22A3 | 61.1 | 0.002 |
| SLCO2B1 | 54.4 | 0.0006 |
| ABCC2 | 35.5 | 0.0003 |
| SLC7A7 | 7.8 | 0.006 |
| AQP7 | 6.4 | 0.03 |
| SLC5A1 | 4.9 | 0.01 |
| SLC28A3 | 2.9 | 0.01 |
| ABCF1 | 2.3 | 0.0008 |
| ATP7A | 2.2 | 0.007 |
| ABCB6 | 2.1 | 0.002 |

### Example 6: Human Drug Transporter Expression—RT-PCR

### Karpas-R Drug Transporter TaqMan RT-PCR

To confirm the upregulation of drug transporter genes potentially implicated in the acquired resistance phenotype, mRNA from Karpas wt, ADCR and PBDR cells were collected and cDNA synthesis performed with SuperScript III RT kit (LifeTech). Rt-PCR reaction mixes were prepared using TaqMan gene expression probes (LifeTech) for the drug transporter of choice or the housekeeping gene ABL-1 and fold change/regulation calculated using ΔΔCT method as described above. The change in expression levels for ABCG2, ABCC2 and SLCO2B1 in Karpas-ADCR and PBDR cells versus wildtype are shown in FIG. 31.

The mean fold change in expression is shown in the table below.

| | Karpas ADC-R fold | Karpas PBD-R fold |
|---|---|---|
| Gene | upregulation | upregulation |
| ABCG2 | 134 | 1.9 |
| ABCC2 | 6.5 | 0.8 |
| SLCO2B1 | 9.5 | 6 |

### NCI-N87-R Drug Transporters TaqMan RT-PCR

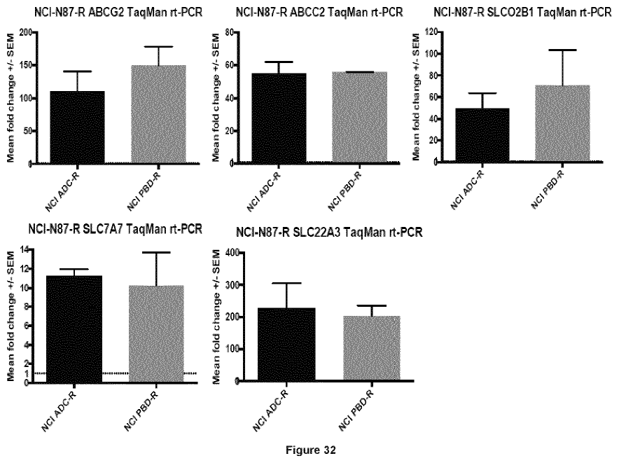

To confirm the upregulation of drug transporter genes potentially implicated in the acquired resistance phenotype, mRNA from NCI-N87 wt, ADCR and PBDR cells were collected and cDNA synthesis performed with SuperScript III RT kit (LifeTech). Rt-PCR reaction mixes were prepared using TaqMan gene expression probes (LifeTech) for the drug transporter of choice or the housekeeping gene ABL-1 and fold change/regulation calculated using ΔΔCT method as described above. The change in expression levels for ABCG2, ABCC2 and SLCO2B1, SLC7A7 and SLC22A3 in NCI-N87-ADCR and PBDR cells versus wildtype are shown in FIG. 32.

The mean fold change in expression is shown in the table below.

| | NCI-N87 ADC-R | NCI-N87 PBD-R |
|---|---|---|
| Gene | fold upregulation | fold upregulation |
| ABCG2 | 111 | 150 |
| ABCC2 | 55 | 56 |
| SLCO2B1 | 49 | 71 |
| SLC7A7 | 11 | 10 |
| SLC22A3 | 220 | 201 |

### DNA Damage Signalling Pathway

In contrast to the results obtained with the human drug transporter gene array, an RT² PCR array for DNA damage signalling pathway showed no significantly upregulated genes in any of the acquired resistant lines. Overall, the data are consistent with the mechanism of resistance being upstream of the DNA damage produced by the PBD dimer.

### Example 7: Human Drug Transporter Expression—Western Blot

### Karpas-R ABC Transporter Western Blot

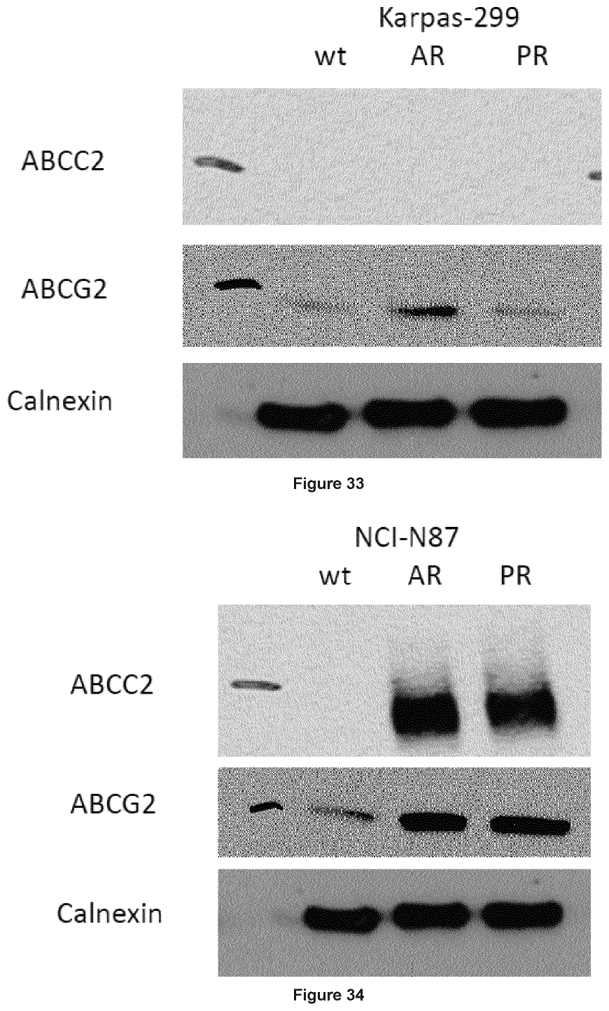

To confirm the drug transporter genes shown to be upregulated by PCR also gave rise to an increase in protein expression, 30-40 μg protein lysate from Karpas resistant and wt cells was loaded into a Mini-Proean TGX 4-15% Tris-Gly gel (BioRad) and run for 120v for 1 hour. The gel was then transferred onto a Trans-Blot Turbo NCL pack (BioRad). The nitrocellulose was then blocked with 5% milk for 30 mins, and incubated with rabbit monoclonal anti-ABCG2 (Abcam) or rabbit monoclonal anti-ABCC2 (Cell Signalling) overnight at 4° C. The membrane was washed with TBST and incubated with HRP-conjugated secondary antibody before developing the signal with the Amersham ECL Western blotting detection reagents and analysis system (GE Healthcare). A western blot of Karpas wt and resistant cell lines probed for ABCC2 and ABCG2 is shown in FIG. 33 showing increased expression of ABCG2 in the ADCR cell line.

### NCI-N87-R ABC Transporter Western Blot

To confirm the drug transporter genes shown to be upregulated also gave rise to an increase in protein expression, 30-40 μg protein lysate from NCI-N87 resistant and wt cells was loaded into a Mini-Proean TGX 4-15% Tris-Gly gel (BioRad) and run for 120v for 1 hour.

The gel was then transferred onto a Trans-Blot Turbo NCL pack (BioRad). The nitrocellulose was then blocked with 5% milk for 30 mins, and incubated with rabbit monoclonal anti-ABCG2 (Abcam) or rabbit monoclonal anti-ABCC2 (Cell Signalling) overnight at 4° C. The membrane was washed with TBST and incubated with HRP-conjugated secondary antibody before developing the signal with the Amersham ECL Western blotting detection reagents and analysis system (GE Healthcare). A western blot of NCI-N87 wt and resistant cell lines probed for ABCC2 and ABCG2 is shown in FIG. 34 showing increased expression of both ABCC2 and ABCG2 in the acquired resistant cell lines.

### Example 8: Antibody Internalisation

### Methodology

To investigate any difference in antibody internalisation in the Karpas acquired resistant cell lines compared to wt, cells were seeded in a poly-L-ornithine coated 96-well plate and allowed to attach at 37° C. HuMax-TAC was incubated with a 3× molar excess of FabFluor pH red antibody internalisation reagent (Essen Bioscience) for 15 mins at room temperature, then a 3× serial dilution set up with cell culture medium. The labelled antibody dilutions were added to the Karpas cells and the plate was immediately transferred to the IncuCyte Zoom, where images were taken at 10× magnification every 2 hours with phase contrast and red fluorescence filters. Mean red object area per well was calculated using the IncuCyte Zoom software.

Similarly, NCI-N87 wt and acquired resistant cells were seeded in a 96-well plate and allowed to attach at 37° C. Trastuzumab was incubated with a 3× molar excess of FabFluor pH red antibody internalisation reagent (Essen Bioscience) for 15 mins at room temperature, then a 3× serial dilution set up with cell culture medium. The labelled antibody dilutions were added to the cells and the plate was immediately transferred to the IncuCyte Zoom, where images were taken at 10× magnification every 2 hours with phase contrast and red fluorescence filters. Mean red object area per well was calculated using the IncuCyte Zoom software.

### Results

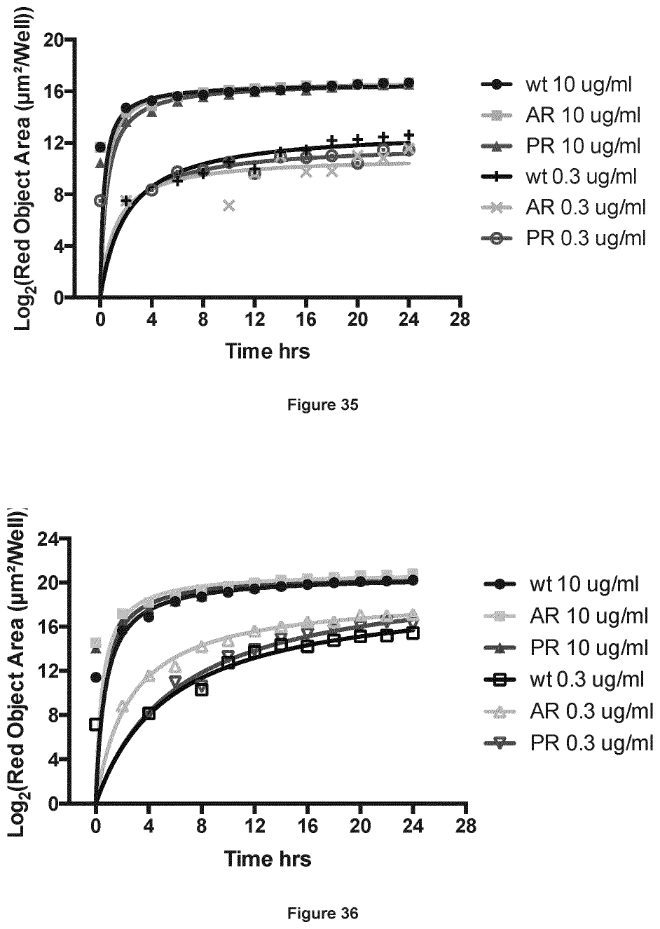

A plot of Karpas wt and resistant cell line CD25 antibody internalisation is shown in FIG. 35.

A plot of NCI-N87 wt and resistant cell line HER2 antibody internalisation is shown in FIG. 36.

In both cases, similar levels and kinetics of internalisation were observed in the resistant lines compared to wildtype.

### Example 9.1: Karpas Resistant Line ABC Inhibitor Combination (MK-571)

### Methodology

To confirm the involvement of ABCC transporters in the acquired resistance to ADCT-301, Karpas ADC and PBD resistant cells were incubated with a non-toxic dose (50 μM) of MK-571 before seeding in a 96-well plate and 96-hour continuous exposure to ADCT-301. The in vitro cytotoxicity assay (MTS) was carried out as previously described.

### Results

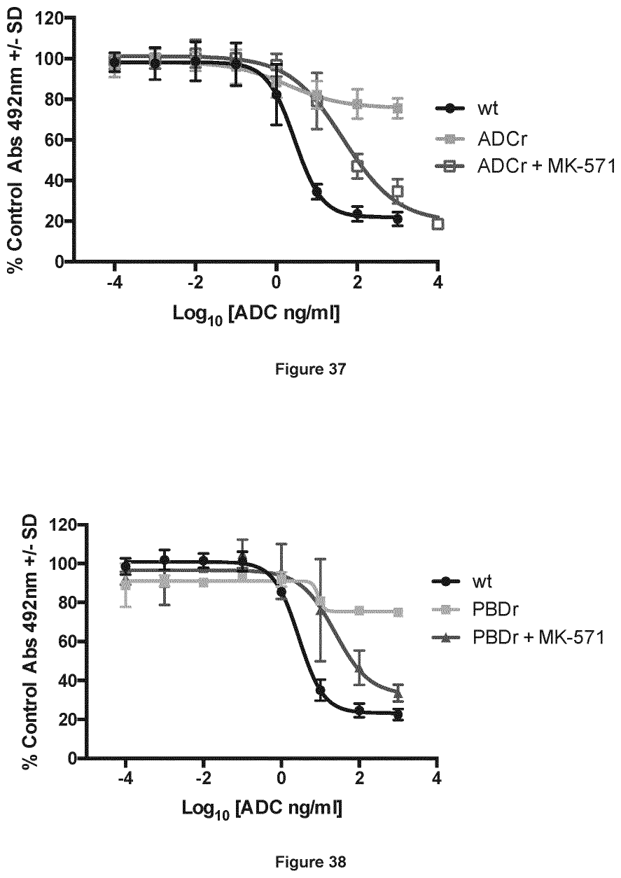

A plot of 96-hour continuous exposure MK-571 and ADCT-301 cytotoxicity of Karpas wt and Karpas ADCR cells is shown in FIG. 37.

A plot of 96-hour continuous exposure MK-571 and ADCT-301 cytotoxicity of Karpas wt and Karpas PBDR cells is shown in FIG. 38.

In both acquired resistant cell lines, addition of the inhibitor was able to partially reverse the resistance.

### Example 9.2: Karpas Resistant Line ABC Inhibitor Combination (Multi-Inhibitor)

### Methodology

Cells were seeded at 10,000 cells per well in a flat bottom 96-well plate, and NCI-N87 cell were incubated overnight to allow for cell attachment.

For ADC growth inhibition assays, cells were then incubated with serial dilutions of ADCT-301 or ADCT-502 in triplicate. For the SG3199 growth inhibition assays, cells were mixed with a serial dilution of SG3199 prepared in DMSO before seeding to ensure the DMSO concentration was the same in all wells and had no effect on cell growth.

Growth inhibition was measured after 96 hours in Karpas-299 cells and 144 hours in NCI-N87 cells using the CellTiter 96 AQueous One MTS Solution (Promega) and absorbance measured on a Multiskan Ascent plate reader (ThermoFisher) at 492 nm.

Growth inhibition was calculated as a percentage of absorbance compared to an untreated control, and IC50 values were calculated using the sigmoidal, 4PL, X is log(concentration) equation in GraphPad Prism.

For drug transporter inhibitor combination cytotox, the resistant cell lines were incubated overnight with either 5 μM MK-571 (ABCC2 inhibitor), 10 μM FTC (ABCG2 inhibitor), 5 μM Reversin-121 (ABCB1 inhibitor) or 10 nM Lovastatin (SLCO2B1 inhibitor) overnight before carrying out the ADC or PBD growth inhibition assay as previously described.

### Results

In order to investigate further the contribution of ABC drug transporter upregulation in the acquired resistance to the PBD dimer and PBD dimer-based ADC resistant cell lines, inhibitors of ABCG2 (Fumitremorgin C, FTC) and ABCC2 (MK-571) were used in combination with the ADC or PBD dimer to assess the effect on growth inhibition using the MTS assay.

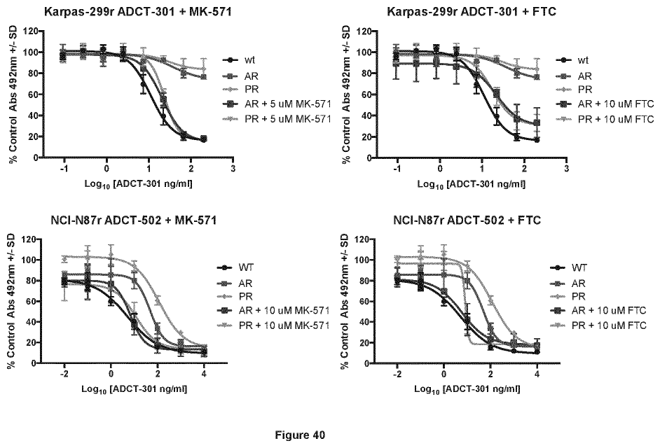

Karpas and NCI-N87 resistant cells were treated with a non-toxic dose of MK-571 (5 μM) or FTC (10 μM) for 24 hours before the addition of the ADC or PBD dimer. Both MK-571 and FTC showed a dramatic re-sensitisation of Karpas-299 ADCr and PBDr cells to ADCT-301 treatment (FIG. 40). Similarly, in the NCI-N87 ADCr and PBDr cells treated with either MK-571 or FTC the response to ADCT-502 was restored to the level of the parental cell line (FIG. 40).

Similar restoration of sensitivity was observed when the ABC transporter inhibitors were combined with SG3199 in Karpas ADCr and PBDr cells (FIG. 41). Lovastatin, an inhibitor of SLCO2B1, however failed to reverse the resistance in any of the cell lines (FIG. 42).

In order to correlate the inhibition of the ABC transporters with the reversal of ADC and PBD dimer resistance mechanistically, the comet assay was used to measure ICL formation in the resistant cell lines by the ADC or PBD dimer pre-treated with either 5 μM MK-571 or 10 μM FTC.

In all the resistant cell lines, treatment with transporter inhibitor resulted in increased formation of ADC or PBD-induced DNA interstrand cross-linking (FIG. 43). These data are consistent with increased retention of PBD dimer in cells resulting in increased DNA damage and resultant cytotoxicity.

In the accompanying figures, each data point represents the average of at least 3 biological repeats with +/−SD error bars. p-values obtained using two-tailed, unpaired t-tests.

### Example 10: Karpas-R Cross-Resistance with SG2000

To measure the cross-resistance of the Karpas ADCR and PBDR cells to the PBD dimer warhead SG2000, the in vitro cytotoxicity (MTS) assay was carried out as previously described in Example 2. The mean IC50 values and fold resistance compared to the wildtype (wt) cell line are shown in the table below.

| Cell line | SG2000 IC50 nM (fold resistance) |
|---|---|
| Karpas wt | 0.9 |
| Karpas ADCr | 2.8 (3.1) |
| Karpas PBDr | 4.7 (5.2) |

### Example 11: NCI-N87-R Cross-Resistance with Other PBD Dimers

### SG3560

To investigate the cross-resistance of the NCI-N87 ADCR and PBDR cells to other PBD dimer containing ADCs, in vitro cytotoxicity was compared to the wt cells treated with the C2-linked PBD-ADC trastuzumab-SG3560, using the same MTS assay protocol previously described.

| | Trastuzumab-SG3560 IC50 ng/mL |
|---|---|
| Cell line | (fold resistance) |
| NCI-N87 wt | 1 |
| NCI-N87 ADCr | 95 (95) |
| NCI-N87 PBDr | 54 (54) |

### SG2000

To measure the cross-resistance of the NCI-N87 ADCR and PBDR cells to the PBD dimer warhead SG2000, the in vitro cytotoxicity (MTS) assay was carried out as previously described. The mean IC50 values and fold resistance compared to the wt cell line are shown in the table below.

| Cell line | SG2000 IC50 pM (fold resistance) |
|---|---|
| NCI-N87 wt | 64 |
| NCI-N87 ADCr | 483 (7.5) |
| NCI-N87 PBDr | 493 (7.7) |

### Example 12: NCI-N87 Resistant Line HER2 Antibody Binding

### Methodology

NCI-N87 wt and resistant cells were blocked at 4° C. for 30 mins before being incubated with a serial dilution series of trastuzumab for 1 hour on ice. The cells were then washed and incubated with a F(ab′)2-Goat anti-Human IgG Fc secondary antibody conjugated to Alexa Fluor 488. After incubation for 1 hour on ice in the dark the cells were washed and run on a Fortessa X20 flow cytometer, and the MFI using the B530/30 laser/filter.

### Results

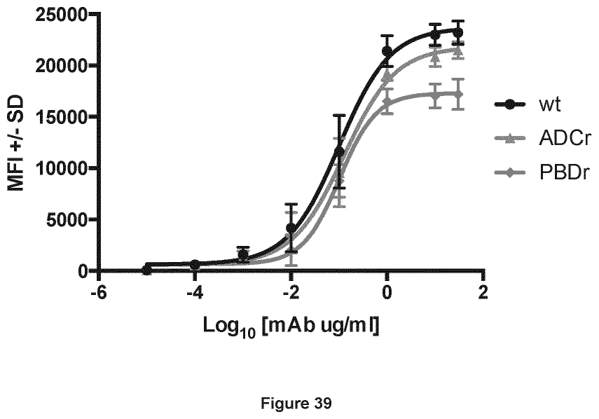

The resulting curves are shown in FIG. 39.

Receptor affinity was similar and a small decrease in peak MFI was observed in the resistant cell lines compared to wt cells, the effect being greatest in the PBDr cell line.

### Example 13: Protein Analysis of ABC Transporters and siRNA Knockout of ABCC2

### Methodology

Silencer® Select siRNA oligonucleotides targeting ABCC2 and nontargeting siRNAs were purchased from Ambion/ThermoFisher. For reverse transfection, Opti-MEM medium was mixed with siRNA to give a final concentration of 25 pmol/L, this was then combined with diluted Lipofectamine® RNAiMAX (Thermo Scientific). After 20-minute incubation at room temperature, the transfection mixture was aliquoted into 6-well plates. Cells were added to each well containing siRNA and RNAiMAX complex. 48 hours after transfection, cells were harvested and used for immunoblotting or growth inhibition assays as previously described.

### Results

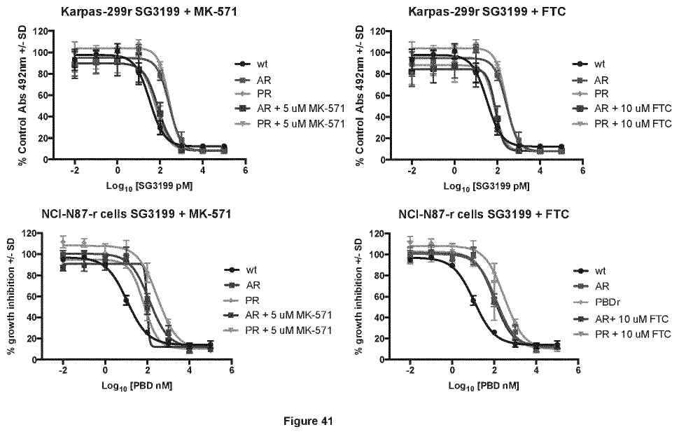

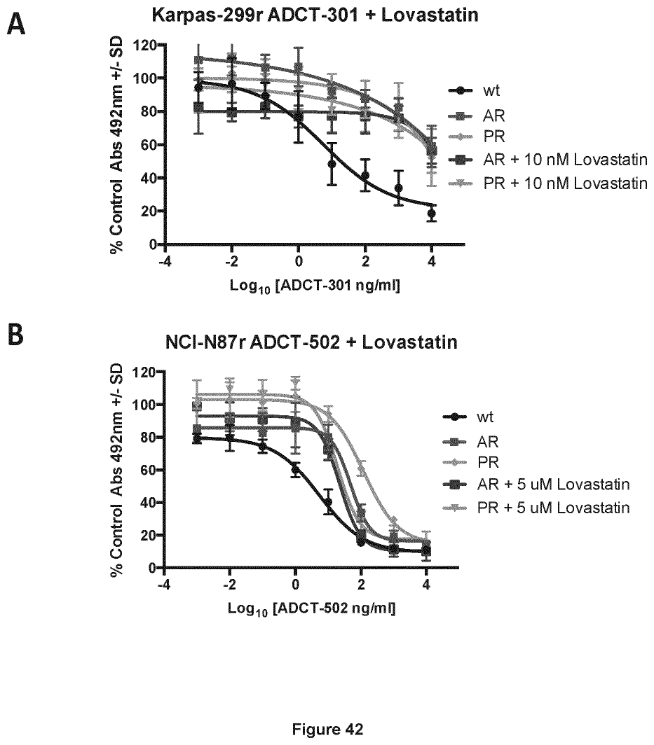

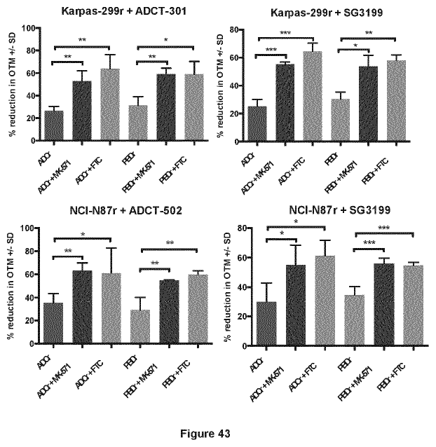

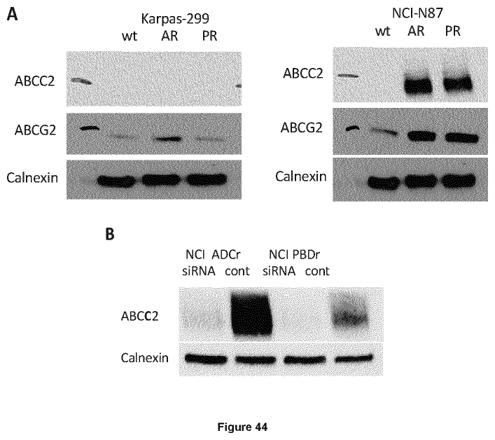

The two drug transporters with the most consistent mRNA upregulation across the resistant cell lines, which also responded to appropriate transporter inhibition to restore drug sensitivity were ABCG2 and ABCC2. Immunoblotting showed the ABCG2 protein to be upregulated in the Karpas-299 ADCr cells compared to the parental cell line, while any upregulation in PBDr cells could not be detected (FIG. 44A). This reflects the very different levels of upregulation observed by PCR in these cells. Despite numerous attempts, ABCC2 protein was could not able to be observed by immunoblotting in either parental nor the resistant Karpas-299 cells. In the NCI-N87 resistant cell line immunoblots, both ABCC2 and ABCG2 were both clearly upregulated compared to the parental cell line (FIG. 44A).

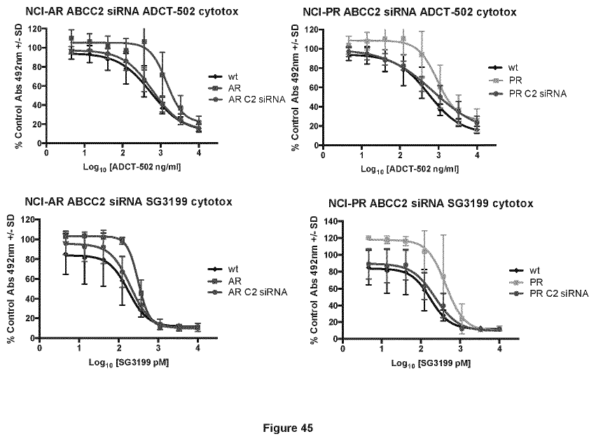

NCI-N87 cells were transfected with siRNA against ABCC2 and ABCG2. ABCG2 was not able to be effectively knocked out due to the very long half-life of the protein (data not shown), but ABCC2 was successfully depleted in both NCI-N87 ADCr and PBDr cells compared with a non-target siRNA control (FIG. 44B). The depletion of ABCC2 in the NCI-N87 ADCr and PBDr cells was able to restore cytotoxic sensitivity to ADCT-502 and SG3199 to the level of the parental cell line (FIG. 45), further implicating ABCC2 in the mechanism of acquired resistance in these cell lines.

SEQUENCES
SEQ ID NO. 1 (AB12 VH):
QVQLVQSGAEVKKPGSSVKVSCKASGGTFSRYIINWVRQAPGQGLEWMGRIIPILGVENYA
QKFQGRVTITADKSTSTAYMELSSLRSEDTAVYYCARKDWFDYWGQGTLVTVSSASTKGP
SVFPLA
SEQ ID NO. 2 (AB12 VL):
EIVLTQSPGTLSLSPGERATLSCRASQSVSSYLAWYQQKPGQAPRLLIYGASSRATGIPDRF
SGSGSGTDFTLTISRLEPEDFAVYYCQQYGSSPLTFGGGTKVEIKRTVAAPSVFIFP
SEQ ID NO. 3 (AB12 VH CDR1):
RYIIN
SEQ ID NO. 4 (AB12 VH CDR2):
RIIPILGVENYAQKFQG
SEQ ID NO. 5 (AB12 VH CDR3):
KDWFDY
SEQ ID NO. 6 (AB12 VL CDR1):
RASQSVSSYLA
SEQ ID NO. 7 (AB12 VL CDR2):
GASSRAT
SEQ ID NO. 8 (AB12 VL CDR3):
QQYGSSPLT
SEQ ID NO. 11 (RB4v1.0 VH):
QVQLVQPGAEVVKPGASVKLSCKTSGYTFTSNWMHWVKQRPGQGLEWIGEIDPSDSYTN
YNQNFKGKAKLTVDKSTSTAYMEVSSLRSDDTAVYYCARGSNPYYYAMDYWGQGTSVTV
S
SEQ ID NO. 12 (RB4v1.2 VH):
QVQLVQPGAEVVKPGASVKLSCKTSGYTFTSNWMHWVKQAPGQGLEWIGEIDPSDSYTN
YNQNFQGKAKLTVDKSTSTAYMEVSSLRSDDTAVYYCARGSNPYYYAMDYWGQGTSVTV
S
SEQ ID NO.13 (RB4v1.0 VK):
EIVLTQSPAIMSASPGERVTMTCSASSGVNYMHWYQQKPGTSPRRWIYDTSKLASGVPAR
FSGSGSGTSYSLTISSMEPEDAATYYCHQRGSYTFGGGTKLEIK
SEQ ID NO. 14 (RB4v1.2 VK):
EIVLTQSPAIMSASPGERVTMTCSASSGVNYMHWYQQKPGTSPRRWIYDTSKLASGVPAR
FSGSGSGTSYSLTISSMEPEDAATYYCHQRGSYTFGGGTKLEIK
SEQ ID NO. 15 (Epratuzumab VH):
QVQLVQSGAEVKKPGSSVKVSCKASGYTFTSYWLHWVRQAPGQGLEWIGYINPRNDYTE
YNQNFKDKATITADESTNTAYMELSSLRSEDTAFYFCARRDITTFYWGQG
SEQ ID NO. 16 (Epratuzumab VL):
DIQLTQSPSSLSASVGDRVTMSCKSSQSVLYSANHKNYLAWYQQKPGKAPKLLIYWASTRE
SGVPSRFSGSGSGTDFTFTISSLQPEDIATYYCHQYLSSVVTFGQG
SEQ ID NO. 17 (EMabC220?HC):
QVQLVQSGAEVKKPGSSVKVSCKASGYTFTSYWLHWVRQAPGQGLEWIGYINPRNDYTEY
NQNFKDKATITADESTNTAYMELSSLRSEDTAFYFCARRDITTFYWGQGTLVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTVPPVPAPELLGGPSVFLFP
PKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPG
SEQ ID NO. 18 (EMabC220-LC):
DIQLTQSPSSLSASVGDRVTMSCKSSQSVLYSANHKNYLAWYQQKPGKAPKLLIYWASTRE
SGVPSRFSGSGSGTDFTFTISSLQPEDIATYYCHQYLSSWTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGES
SEQ ID NO. 21 (J591 VH):
EVQLQQSGPELKKPGTSVRISCKTSGYTFTEYTIHWVKQSHGKSLEWIGNINPNNGGTTYN
QKFEDKATLTVDKSSSTAYMELRSLTSEDSAVYYCAAGWNFDYWGQGTTLTVSS
SEQ ID NO. 22 (J591 VL):
DIVMTQSHKFMSTSVGDRVSIICKASQDVGTAVDWYQQKPGQSPKLLIYWASTRHTGVPD
RFTGSGSGTDFTLTITNVQSEDLADYFCQQYNSYPLTFGAGTMLDLK
SEQ ID NO. 23 (J591 VH Delm):
EVQLVQSGPEVKKPGATVKISCKTSGYTFTEYTIHWVKQAPGKGLEWIGNINPNNGGTTYN
QKFEDKATLTVDKSTDTAYMELSSLRSEDTAVYYCAAGWNFDYWGQGTLLTVSS
SEQ ID NO. 24 (J591 VK Delm):
DIQMTQSPSSLSTSVGDRVTLTCKASQDVGTAVDWYQQKPGPSPKLLIYWASTRHTGIPSR
FSGSGSGTDFTLTISSLQPEDFADYYCQQYNSYPLTFGPGTKVDIK
SEQ ID NO. 25 (J591BJ Delm heavy chain):
EVQLVQSGPEVKKPGATVKISCKTSGYTFTEYTIHWVKQAPGKGLEWIGNINPNNGGTTYN
QKFEDKATLTVDKSTDTAYMELSSLRSEDTAVYYCAAGWNFDYWGQGTLLTVSSASTKGP
SVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTVPPVPAPELLGGPSVFLFPPK
PKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVM
HEALHNHYTQKSLSLSPG
SEQ ID NO. 26 (J591BJ DeIm light chain):
DIQMTQSPSSLSTSVGDRVTLTCKASQDVGTAVDWYQQKPGPSPKLLIYWASTRHTGIPSR
FSGSGSGTDFTLTISSLQPEDFADYYCQQYNSYPLTFGPGTKVDIKRTVAAPSVFIFPPSDE
QLKSGTASVVCLLNNFYPREAKVQWKVDNALQSGNSQESVTEQDSKDSTYSLSSTLTLSK
ADYEKHKVYACEVTHQGLSSPVTKSFNRGES
SEQ ID NO.31 [1H12 VH, CDR underline]
QVQLVESGGGVVQPGRSLRLSCAASGFTFSSYGMSWVRQAPGKGLEWVATISSGGSYTY
YPDSVKGRFTISRDNSKNTLYLQMNSLRAEDTAVYYCARHPIYYTYDDTMDYWGQGTTVT
VSS
SEQ ID NO.32 [1H12 VL, CDR underline]
EIVLTQSPGTLSLSPGERATLSCSASSSVSSGNFHWYQQKPGLAPRLLIYRTSNLASGIPAR
FSGSGSGTDFTLTISSLEPEDFAVYYCQQWSGYPWTFGGGTKLEIK
SEQ ID NO.33 [1H12 Heavy Chain]
QVQLVESGGGVVQPGRSLRLSCAASGFTFSSYGMSWVRQAPGKGLEWVATISSGGSYTY
YPDSVKGRFTISRDNSKNTLYLQMNSLRAEDTAVYYCARHPIYYTYDDTMDYWGQGTTVT
VSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQY
N*STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPG
N* indicates Asn297 (numbering according to Kabat)
SEQ ID NO.34 [1H12 Light Chain]
EIVLTQSPGTLSLSPGERATLSCSASSSVSSGNFHWYQQKPGLAPRLLIYRTSNLASGIPAR
FSGSGSGTDFTLTISSLEPEDFAVYYCQQWSGYPWTFGGGTKLEIKRTVAAPSVFIFPPSDE
QLKSGTASVVCLLNNFYPREAKVQWKVDNALQSGNSQESVTEQDSKDSTYSLSSTLTLSK
ADYEKHKVYACEVTHQGLSSPVTKSFNRGEC
SEQ ID NO.35 [1H12 VH CDR]
SYGMS
SEQ ID NO.36 [1H12 VH CDR2]
TISSGGSYTYYPDSVKG
SEQ ID NO.37 [1H12 VH CDR3]
HPIYYTYDDTMDY
SEQ ID NO.38 [1H12 VL CDR]
SASSSVSSGNFH
SEQ ID NO.39 [1H12 VL CDR2]
RTSNLAS
SEQ ID NO.40 [1H12 VL CDR3]
QQWSGYPWT
SEQ ID NO.41 [HuBa-1-3d VH, CDR underline]
QVQLVQSGAEVKKPGASVKVSCKGSGYTFTDYAMHWVRQAPGQGLEWIGVISTYYGNTN
YNQKFKGKATMTVDKSTSTAYMELRSLRSDDTAVYYCARGGLREYYYAMDYWGQGTMVT
VSS
SEQ ID NO.42 [HuBa-1-3d VL, CDR underline]
DIVMTQSPDSLAVSLGERATINCKSSQSLLNSSNQKNYLAWYQQKPGQPPKLLVYFASTRE
SGVPDRFSGSGSGTDFTLTISSLQAEDVAVYYCQQHYSTPPTFGQGTKLEIK
SEQ ID NO.43 [HuBa-1-3d Heavy Chain]
QVQLVQSGAEVKKPGASVKVSCKGSGYTFTDYAMHWVRQAPGQGLEWIGVISTYYGNTN
YNQKFKGKATMTVDKSTSTAYMELRSLRSDDTAVYYCARGGLREYYYAMDYWGQGTMVT
VSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQY
N*STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPG
N* indicates Asn297
SEQ ID NO.44 [HuBa-1-3d Light Chain]
DIVMTQSPDSLAVSLGERATINCKSSQSLLNSSNQKNYLAWYQQKPGQPPKLLVYFASTRE
SGVPDRFSGSGSGTDFTLTISSLQAEDVAVYYCQQHYSTPPTFGQGTKLEIKRTVAAPSVFI
FPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO.45 [HuBa-1-3d VH CDR1]
DYAMH
SEQ ID NO.46 [HuBa-1-3d VH CDR2]
VISTYYGNTNYNQKFKG
SEQ ID NO.47 [HuBa-1-3d VH CDR3]
GGLREYYYAMDY
SEQ ID NO.48 [HuBa-1-3d VL CDR]
KSSQSLLNSSNQKNYLA
SEQ ID NO.49 [HuBa-1-3d VL CDR2]
FASTRES
SEQ ID NO.50 [HuBa-1-3d VL CDR3]
QQHYSTPPT
SEQ ID NO.51 [HuBa-1-3d Heavy Chain, terminal K]
QVQLVQSGAEVKKPGASVKVSCKGSGYTFTDYAMHWVRQAPGQGLEWIGVISTYYGNTN
YNQKFKGKATMTVDKSTSTAYMELRSLRSDDTAVYYCARGGLREYYYAMDYWGQGTMVT
VSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQY
N*STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
N* indicates Asn297
SEQ ID NO.61 [3A4 VH, CDR underline]
QIQLVQSGAEVKKPGASVKVSCKASGYTFTDDYMSWVKQAPGQGLEWIGDINPYNGDTNY
NQKFKGKATLTVDKSTSTAYMELSSLRSEDTAVYYCARDPGAMDYWGQGTLVTVSS
SEQ ID NO.62 [3A4 VL, CDR underline]
DIVMTQTPLSLPVTPGEPASISCRSSQSLLHSNGNTYLEWYLQKPGQSPQLLIYTVSNRFSG
VPDRFSGSGSGTDFTLKISRVEAEDVGVYYCFQGSHVPLTFGQGTKLEIK
SEQ ID NO.63 [3A4 Heavy Chain]
QIQLVQSGAEVKKPGASVKVSCKASGYTFTDDYMSWVKQAPGQGLEWIGDINPYNGDTNY
NQKFKGKATLTVDKSTSTAYMELSSLRSEDTAVYYCARDPGAMDYWGQGTLVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLF
PPKPKDTLMISRTPEVTCWVDVSHEDPEVKFNWYVDGVEVHNAKTKPREEQYN*STYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSC
SVMHEALHNHYTQKSLSLSPG
N* indicates Asn297
SEQ ID NO.64 [3A4 Light Chain]
DIVMTQTPLSLPVTPGEPASISCRSSQSLLHSNGNTYLEWYLQKPGQSPQLLIYTVSNRFSG
VPDRFSGSGSGTDFTLKISRVEAEDVGVYYCFQGSHVPLTFGQGTKLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO.65 [3A4 VH CDR]
GYTFTDDYMS
SEQ ID NO.66 [3A4 VH CDR2]
DINPYNGDTN
SEQ ID NO.67 [3A4 VH CDR3]
DPGAMDY
SEQ ID NO.68 [3A4 VL CDR1]
RSSQSLLHSNGNTYLE
SEQ ID NO.69 [3A4 VL CDR2]
TVSN RFS
SEQ ID NO.70 [3A4 VL CDR3]
FQGSHVPLT
SEQ ID NO.71 [3A4 Heavy Chain, terminal K]
QIQLVQSGAEVKKPGASVKVSCKASGYTFTDDYMSWVKQAPGQGLEWIGDINPYNGDTNY
NQKFKGKATLTVDKSTSTAYMELSSLRSEDTAVYYCARDPGAMDYWGQGTLVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLF
PPKPKDTLMISRTPEVTCWVDVSHEDPEVKFNWYVDGVEVHNAKTKPREEQYN*STYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSC
SVMHEALHNHYTQKSLSLSPGK
N* indicates Asn297
SEQ ID NO.72 [Human KAAG1]
MDDDAAPRVEGVPVAVHKHALHDGLRQVAGPGAAAAHLPRWPPPQLAASRREAPPLSQR
PHRTQGAGSPPETNEKLTNPQVKEK
SEQ ID NO.73 [3A4-L2 VL, CDR underline]
DVVMTQTPLSLPVTPGEPASISCRSSQSLLHSNGNTYLEWYLQKPGQSPKLLIYTVSNRFS
GVPDRFSGSGSGTDFTLKISRVEAEDVGVYYCFQGSHVPLTFGQGTKLEIK
SEQ ID NO.74 [3A4-L2 Light Chain]
DVVMTQTPLSLPVTPGEPASISCRSSQSLLHSNGNTYLEWYLQKPGQSPKLLIYTVSNRFS
GVPDRFSGSGSGTDFTLKISRVEAEDVGVYYCFQGSHVPLTFGQGTKLEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO.75 [3A4-K4 VL, CDR underline]
DIVMTQSPDSLAVSLGERATINCRSSQSLLHSNGNTYLEWYQQKPGQPPKLLIYTVSNRFS
GVPDRFSGSGSGTDFTLTISSLQAEDVAVYYCFQGSHVPLTFGQGTKVEIK
SEQ ID NO.76 [3A4-K4 Light Chain]
DIVMTQSPDSLAVSLGERATINCRSSQSLLHSNGNTYLEWYQQKPGQPPKLLIYTVSNRFS
GVPDRFSGSGSGTDFTLTISSLQAEDVAVYYCFQGSHVPLTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO.81 [XA4 VH, CDR underline]
QVHLVESGGGVVQPGRSLRLSCVASGITFRIYGMHWVRQAPGKGLEWVAVLWYDGSHEY
YADSVKGRFTISRDNSKNTLYLQMNSLRAEDTAIYYCARDGDYYDSGSPLDYWGQGTLVT
VSS
SEQ ID NO.82 [XA4 VL, CDR underline]
EIVLTQSPATLSLSPGERATLSCRASQSVSSYLAWYQQKPGQAPRLLIYDASNRATGIPA
RFSGSGSGTDFTLTISSLEPEDFAVYYCQQRSNWPLTFGGGTKVEIK
SEQ ID NO.83 [XA4 Heavy Chain]
QVHLVESGGGVVQPGRSLRLSCVASGITFRIYGMHWVRQAPGKGLEWVAVLWYDGSHEY
YADSVKGRFTISRDNSKNTLYLQMNSLRAEDTAIYYCARDGDYYDSGSPLDYWGQGTLVT
VSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQY
N*STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPG
N* indicates Asn297
SEQ ID NO.84 [XA4 Light Chain]
EIVLTQSPATLSLSPGERATLSCRASQSVSSYLAWYQQKPGQAPRLLIYDASNRATGIPARF
SGSGSGTDFTLTISSLEPEDFAVYYCQQRSNWPLTFGGGTKVEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC
SEQ ID NO.91 [XA4 Heavy Chain, terminal K]
QVHLVESGGGVVQPGRSLRLSCVASGITFRIYGMHWVRQAPGKGLEWVAVLWYDGSHEY
YADSVKGRFTISRDNSKNTLYLQMNSLRAEDTAIYYCARDGDYYDSGSPLDYWGQGTLVT
VSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQY
N*STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
N* indicates Asn297
SEQ ID NO.92 [XFT VH, CDR underline]
QVELVQSGAVKKPGESLKISCKGSGYSFTSYWIGWVRQAPGKGLEWMGIIDPGDSRTRYS
PSFQGQVTISADKSISTAYLQWSSLKASDTAMYYCARGQLYGGTYMDGWGQGTLVTVSS
SEQ ID NO.93 [XFT VL, CDR underline]
DIALTQPASVSGSPGQSITISCTGTSSDIGGYNSVSWYQQHPGKAPKLMIYGVNNRPSGVS
NRFSGSKSGNTASLTISGLQAEDEADYYCSSYDIESATPVFGGGTKLEIK
SEQ ID NO.94 [XFT Heavy Chain]
QVELVQSGAVKKPGESLKISCKGSGYSFTSYWIGWVRQAPGKGLEWMGIIDPGDSRTRYS
PSFQGQVTISADKSISTAYLQWSSLKASDTAMYYCARGQLYGGTYMDGWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGN
VFSCSVMHEALHNHYTQKSLSLSPG
N* indicates Asn297
SEQ ID NO.95 [XFT Light Chain]
DIALTQPASVSGSPGQSITISCTGTSSDIGGYNSVSWYQQHPGKAPKLMIYGVNNRPSGVSN
RFSGSKSGNTASLTISGLQAEDEADYYCSSYDIESATPVFGGGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO.102 [XFT Heavy Chain, terminal K]
QVELVQSGAVKKPGESLKISCKGSGYSFTSYWIGWVRQAPGKGLEWMGIIDPGDSRTRYS
PSFQGQVTISADKSISTAYLQWSSLKASDTAMYYCARGQLYGGTYMDGWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGN
VFSCSVMHEALHNHYTQKSLSLSPGK
N* indicates Asn297
SEQ ID NO.103 [X09 VH, CDR underline]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGSGTPVTVSS
SEQ ID NO.104 [X09 VL, CDR underline]
DIELTQSPAIMSASPGEKVTMTCSASSSVSYMHWYQQKSGTSPKRWIYDTSKLASGVPGR
FSGSGSGNSYSLTISSVEAEDDATYYCQQWSKHPLTFGSGTKVEIK
SEQ ID NO.105 [X09 Heavy Chain]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGSGTPVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGN
VFSCSVMHEALHNHYTQKSLSLSPG
N* indicates Asn297
SEQ ID NO.106 [X09 Light Chain]
DIELTQSPAIMSASPGEKVTMTCSASSSVSYMHWYQQKSGTSPKRWIYDTSKLASGVPGR
FSGSGSGNSYSLTISSVEAEDDATYYCQQWSKHPLTFGSGTKVEIKRTVAAPSVFIFPPSDE
QLKSGTASVVCLLNNFYPREAKVQWKVDNALQSGNSQESVTEQDSKDSTYSLSSTLTLSK
ADYEKHKVYACEVTHQGLSSPVTKSFNRGEC
SEQ ID NO.113 [X09 Heavy Chain, terminal K]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGSGTPVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGN
VFSCSVMHEALHNHYTQKSLSLSPGK
N* indicates Asn297
SEQ ID NO.114 [X09.2 VH, CDR underline]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGQGTTVTVSS
SEQ ID NO.115 [X09.2 VL, CDR underline]
DIELTQSPAIMSASPGEKVTMTCSASSSVSYMHWYQQKSGTSPKRWIYDTSKLASGVPGR
FSGSGSGNSYSLTISSVEAEDDATYYCQQWSGYPLTFGAGTKLEIK
SEQ ID NO.116 [X09.2 Heavy Chain]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGSGTPVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGN
VFSCSVMHEALHNHYTQKSLSLSPG
N* indicates Asn297
SEQ ID NO.117 [X09.2 Light Chain]
DIELTQSPAIMSASPGEKVTMTCSASSSVSYMHWYQQKSGTSPKRWIYDTSKLASGVPGR
FSGSGSGNSYSLTISSVEAEDDATYYCQQWSKHPLTFGSGTKVEIKRTVAAPSVFIFPPSDE
QLKSGTASVVCLLNNFYPREAKVQWKVDNALQSGNSQESVTEQDSKDSTYSLSSTLTLSK
ADYEKHKVYACEVTHQGLSSPVTKSFNRGEC
SEQ ID NO.118 [X09.2 Heavy Chain, terminal K]
QVQLQQSGPELEKPGASVKISCKASGYSFTGYTMNWVKQSHGKSLEWIGLITPYNGASSY
NQKFRGKATLTVDKSSSTAYMDLLSLTSEDSAVYFCARGGYDGRGFDYWGSGTPVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYN*STY
RVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGN
VFSCSVMHEALHNHYTQKSLSLSPGK
N* indicates Asn297

1-101. (canceled)

102. A method of treating a proliferative disease in a subject, wherein the proliferative disease is resistant to a PBD agent, the method comprising administering to the subject an antagonist of one or more PBD-resistance genes in combination with a therapeutically effective amount of the PBD agent.

103. The method of claim 102, wherein the subject is selected for treatment by a method comprising the steps of:
(a) determining whether one or more PBD-resistance genes are overexpressed in a sample from the subject, and
(b) selecting the subject for treatment with the PBD agent if overexpression of the one or more PBD-resistance genes is not detected in the sample.

104. A method of reducing the resistance of a proliferative cell to a PBD-agent, the method comprising contacting the proliferative cell with an antagonist of one or more PBD-resistance genes.

105. A method for determining whether a proliferative disease in a subject is resistant to treatment with a pyrrolobenzodiazepine (PBD) agent,
the method comprising determining whether one or more PBD-resistance genes are overexpressed in a sample from the subject,
wherein overexpression of the one or more PBD-resistance genes indicates that the proliferative disease is resistant to treatment with the PBD agent.

106. The method of claim 102, wherein the antagonist is an siRNA, or an miRNA, such as miR-200c, miR-212, miR-328, miR-519c, miR-520h, miR-297, or miR-379.

107. The method of claim 102, wherein the antagonist is selected from the group consisting of: MK-571, Biricodar, Probenecid, Reversan, Fumitremorgin C, and Ko143.

108. The method of claim 102, wherein the antagonist is an ABCC2 inhibitor selected from the group consisting of: probenecid, furosemide, ritonavir, saquinavir, lamivudine, abacavir, emtricitabine, efavirenz, delavirdine, nevirapine, cidofovir, adefovir, tenofovir, cyclosporine, PSC833, and MK571.

109. The method of claim 102, wherein the antagonist is an ABCG2 inhibitor selected from the group consisting of: febuxostat, Fumitremorgin C, elacridar, tariquidar, and Ko143.

110. The method of claim 102, wherein the one or more PBD-resistance genes are selected from the group consisting of:
(i) ABCG2, ABCC2, SLCO2B1, SLC7A7, SLC22A3, SLCO2A1, ABCC12, ATP7A, AQP7, SLC5A1, SLC16A2, SLC7A9, ABCB4, ABCC11, ABCF1, SLC28A3, and ABCB6;
(ii) ABCG2, ABCC2, SLCO2B1, SLC7A7, SLC22A3, SLCO2A1, ABCC12, ATP7A, AQP7, and SLC5A1;
(iii) ABCG2, ABCC2, SLCO2B1, SLC7A7, and SLC22A3;
(iv) ABCG2, ABCC2, SLCO2B1, and SLC7A7;
(v) ABCG2, ABCC2, and SLCO2B1;
(vi) ABCG2, ABCC2, and SLC7A7;
(vii) ABCG2, ABCC2, and SLC22A3;
(viii) ABCG2 and ABCC2;
(ix) ABCG2 and SLCO2B1;
(x) ABCG2; and
(xi) ABCC2.

111. The method of claim 103, wherein the method comprises determining whether two, three, four, or five or more PBD-resistance genes are overexpressed in the sample.

112. The method of claim 103, wherein determining PBD-resistance gene overexpression comprises measuring the level of mRNA transcription from the one or more PBD-resistance genes and/or measuring the level of PBD-resistance polypeptide expression.

113. The method of claim 103, wherein overexpression is indicated by an at least 2-fold, 5-fold, 10-fold, 20-fold, 50-fold, or 100-fold increase relative to a control sample.

114. The method of claim 103, wherein overexpression is indicated by an increase relative to a control sample that has a p-value no greater than 0.05, 0.01, 0.005, 0.001, 0.0005, 0.0001, 0.00005, or 0.00001.

115. The method of claim 102, wherein the proliferative disease is cancer.

116. The method of claim 115, wherein the cancer is:
(i) a benign, pre malignant, or malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis;
(ii) a solid tumour;
(iii) a solid tumour associated with CD25+ve infiltrating cells; (iv) a solid tumour associated with CD25+ve infiltrating cells, wherein the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer;
(iv) lymphoma or leukaemia; or
(v) selected from:
Hodgkin's Lymphoma; non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

117. The method of claim 102, wherein the PBD agent is a conjugate of formula L-(D^{L})ₚ, where D^{L} is of formula I or II: 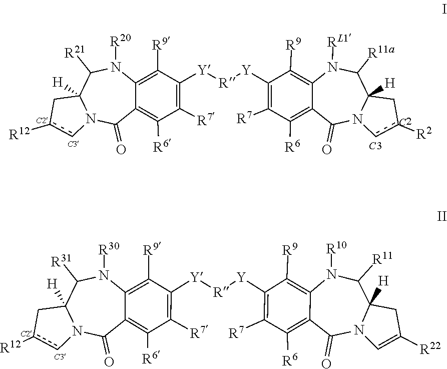
wherein:
L is a cell binding agent (CBA) such as an antibody;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 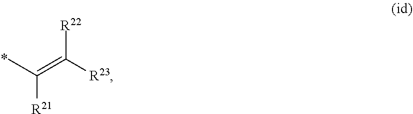
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 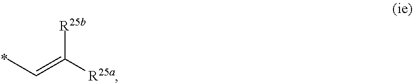
wherein one of R^{25a} and R^{25b} is H and the other is selected from: phenyl, which phenyl is optionally substituted by a group selected from halo, methyl, methoxy; pyridyl; and thiophenyl; and 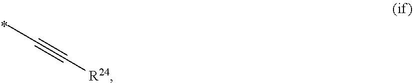
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 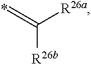
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
R^{L1′} is a linker for connection to the cell binding agent (CBA);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 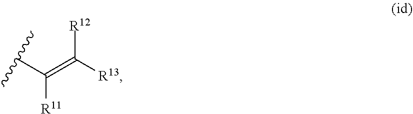
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 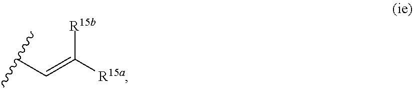
wherein one of R^{15a} and R^{15b} is H and the other is selected from: phenyl, which phenyl is optionally substituted by a group selected from halo, methyl, methoxy; pyridyl; and thiophenyl; and 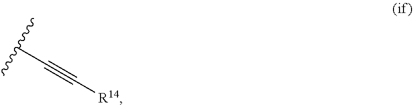
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 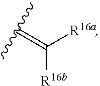
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
R²² is of formula IIIa, formula IIIb or formula IIIc: 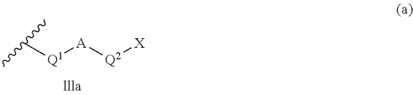
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 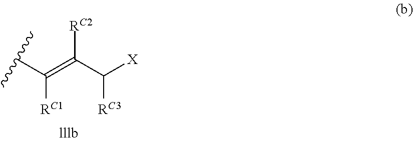
where;
R^{C1}, R^{C2} and R^{C3} are independently selected from H and unsubstituted C₁₋₂ alkyl; 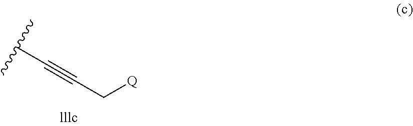
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 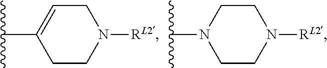
NR^{N}R^{L2′}, wherein R^{N} is selected from the group comprising H and C₁₋₄ alkyl;
R^{L2′} is a linker for connection to the cell binding agent (CBA);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

118. The method of claim 117, wherein the PBD agent comprises a compound of the formula: 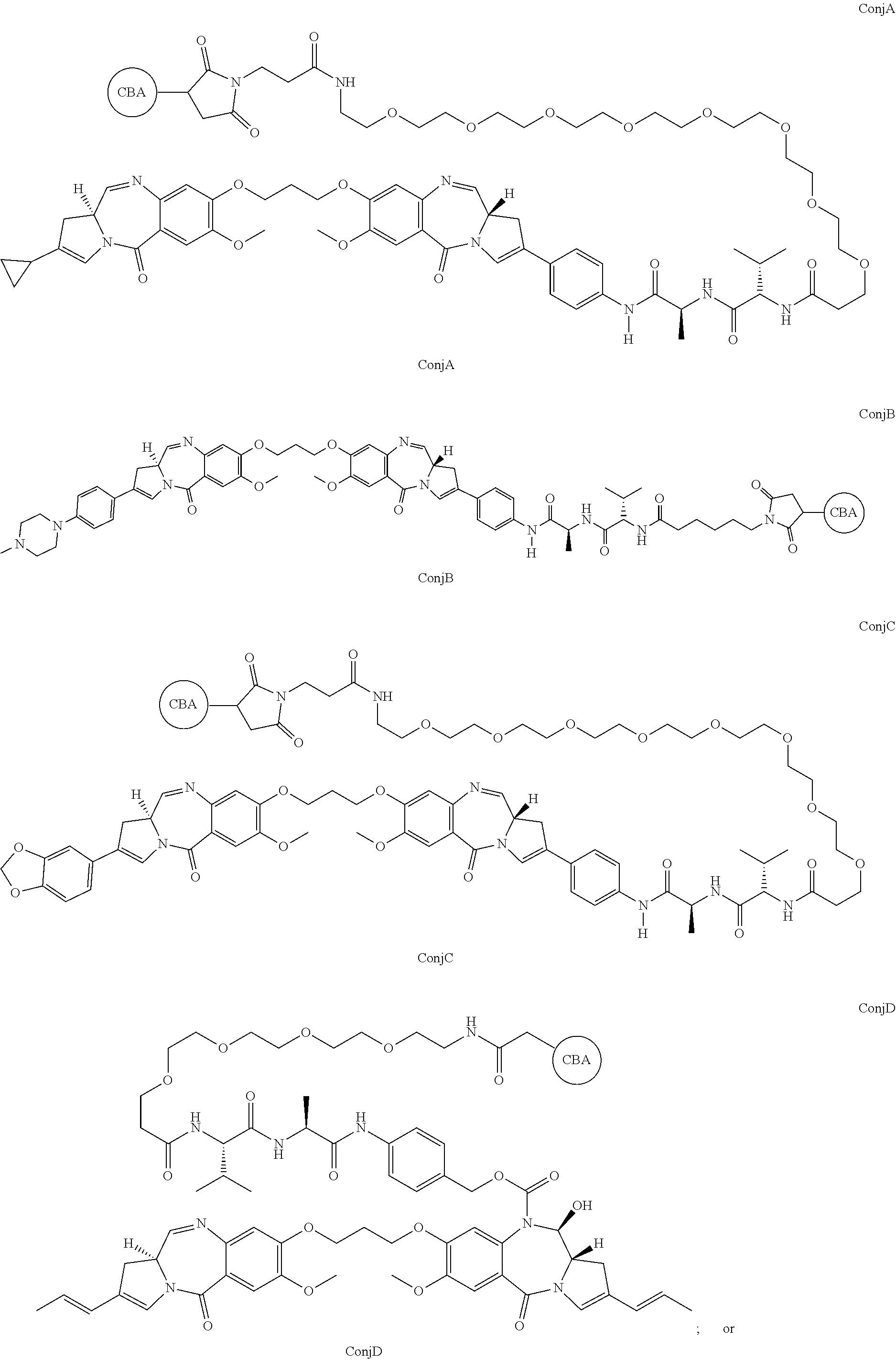 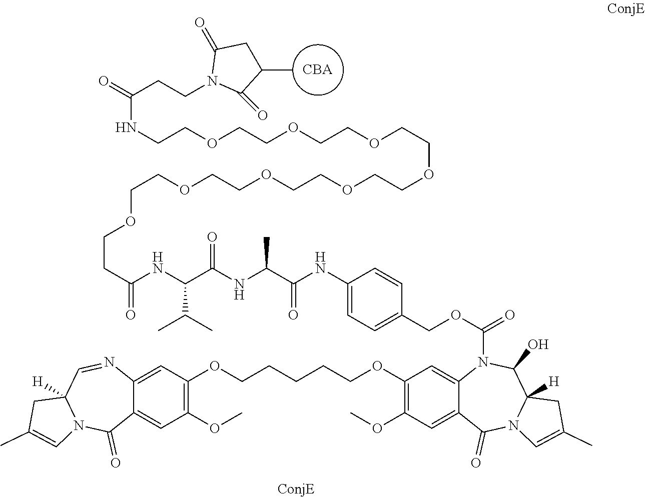
wherein CBA is a cell binding agent such as an antibody.

119. The method of claim 102, wherein the PBD agent is a compound of the formula (III):
L-(DL)ₚ (III)
wherein:
L is a cell binding agent (CBA) such as an antibody;
DL is 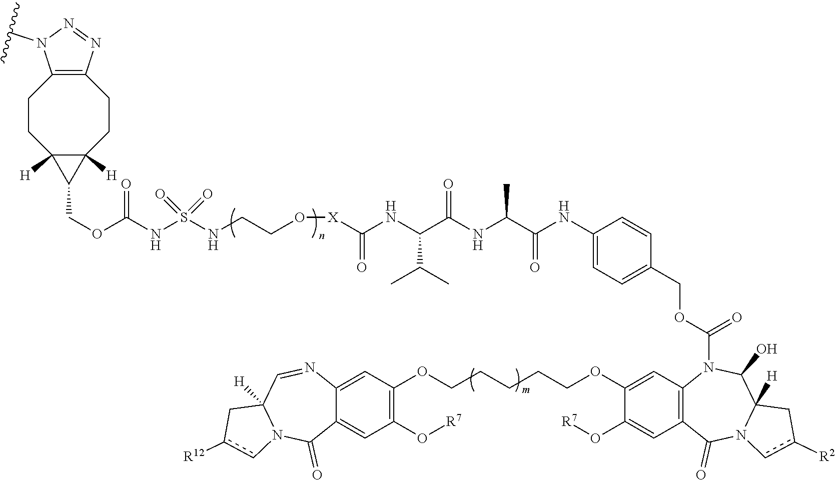
wherein:
X is selected from the group comprising: a single bond, —CH₂— and —C₂H₄—;
n is from 1 to 8;
m is 0 or 1;
R⁷ is either methyl or phenyl;
when there is a double bond between C2 and C3, R² is selected the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 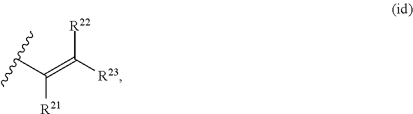
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R² group is no more than 5; 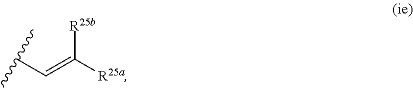
wherein one of R^{25a} and R^{25b} is H and the other is selected from: phenyl, which phenyl is optionally substituted by a group selected from halo, methyl, methoxy; pyridyl; and thiophenyl; and 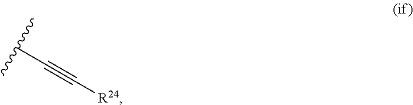
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2 and C3, R² is 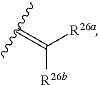
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
when there is a double bond between C2′ and C3′, R¹² is selected the group consisting of:
(iia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl; 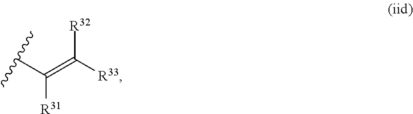
wherein each of R³¹, R³² and R³³ are independently selected from H, C₁₋₃ saturated alkyl, C₂₋₃ alkenyl, C₂₋₃ alkynyl and cyclopropyl, where the total number of carbon atoms in the R¹² group is no more than 5; 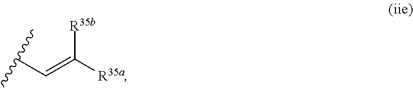
wherein one of R^{35a} and R^{35b} is H and the other is selected from: phenyl, which phenyl is optionally substituted by a group selected from halo, methyl, methoxy; pyridyl; and thiophenyl; and 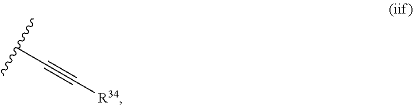
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond between C2′ and C3′, R¹² is 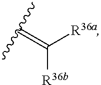
where R^{36a} and R^{36b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{36a} and R^{36b} is H, the other is selected from nitrile and a C₁₋₄ alkyl ester;
and p is from 1 to 8.

120. The method of claim 119, wherein DL is:

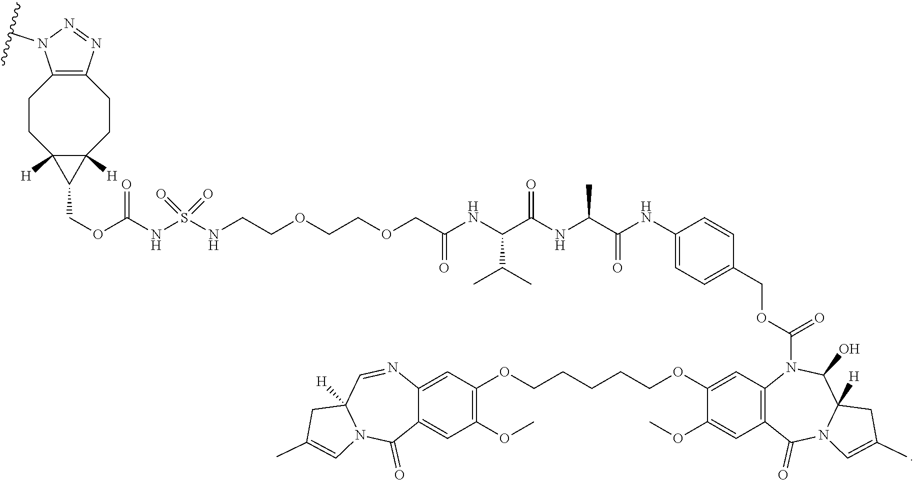 [SMILES: *n1nnc2c1CC[C@@H]1[C@H](CC2)[C@@H]1COC(=O)NS(=O)(=O)NCCOCCOCC(=O)N[C@H](C(=O)N[C@@H](C)C(=O)Nc1ccc(COC(=O)N2c3cc(OCCCCCOc4cc5c(cc4OC)C(=O)N4C=C(C)C[C@H]4C=N5)c(OC)cc3C(=O)N3C=C(C)C[C@H]3[C@@H]2O)cc1)C(C)C]

121. The method of claim 102, wherein the PBD agent is selected from ADCT-301, ADCT-401, ADCT-402, ADCT-602, ADCT-601, or ADCT-701.
